SEQUENCE LISTING (6 sequences)

SEQ ID NO: 1 - protein, 1410 aa, Artificial Sequence
  21E10 (mouse anti-Human)
ATGGGTTGGAGCTGTATCATCTTCTTTCTGGTAGCAACAGCTACAGGTGTGCACTCCCAGGTCCAGCTGCAGCAGTCTGGGCCTGAGCTGGTGAGGCCTGGGGTCTCAGTGAAGATTTCCTGCAAGGGTTCCGGCTACACATTCACTGATTATGCTATGCACTGGGTGAAGCAGAGTCATGCAAAGAGTCTAGAGTGGATTGGAGTTATTAGTACTTACTCTAGTAATACAAACTACAACCAGAAGTTTAAGGGCAAGGCCACAATGACTGTAGACAAATCCTCCAGCACAGCCTATATGGAACTTGCCAGATTGACATCTGAGGATTCTGCCATCTATTACTGTGCAAGAGATGATGGTCACTACGTCTATGCTATGGACTACTGGGGTCAAGGAACCTCAGTCACCGTCTCCTCAGCCAAAACAACAGCCCCATCGGTCTATCCACTGGCCCCTGTGTGTGGAGATACAACTGGCTCCTCGGTGACTCTAGGATGCCTGGTCAAGGGTTATTTCCCTGAGCCAGTGACCTTGACCTGGAACTCTGGATCCCTGTCCAGTGGTGTGCACACCTTCCCAGCTGTCCTGCAGTCTGACCTCTACACCCTCAGCAGCTCAGTGACTGTAACCTCGAGCACCTGGCCCAGCCAGTCCATCACCTGCAATGTGGCCCACCCGGCAAGCAGCACCAAGGTGGACAAGAAAATTGAGCCCAGAGGGCCCACAATCAAGCCCTGTCCTCCATGCAAATGCCCAGCACCTAACCTCTTGGGTGGACCATCCGTCTTCATCTTCCCTCCAAAGATCAAGGATGTACTCATGATCTCCCTGAGCCCCATAGTCACATGTGTGGTGGTGGATGTGAGCGAGGATGACCCAGATGTCCAGATCAGCTGGTTTGTGAACAACGTGGAAGTACACACAGCTCAGACACAGACCCATAGAGAGGATTACAACAGTACTCTCCGGGTGGTCAGTGCCCTCCCCATCCAGCACCAGGACTGGATGAGTGGCAAGGAGTTCAAATGCAAGGTCAACAACAAAGACCTCCCAGCGCCCATCGAGAGAACCATCTCAAAACCCAAAGGGTCAGTAAGAGCTCCACAGGTATATGTCTTGCCTCCACCAGAAGAAGAGATGACTAAGAAACAGGTCACTCTGACCTGCATGGTCACAGACTTCATGCCTGAAGACATTTACGTGGAGTGGACCAACAACGGGAAAACAGAGCTAAACTACAAGAACACTGAACCAGTCCTGGACTCTGATGGTTCTTACTTCATGTACAGCAAGCTGAGAGTGGAAAAGAAGAACTGGGTGGAAAGAAATAGCTACTCCTGTTCAGTGGTCCACGAGGGTCTGCACAATCACCACACGACTAAGAGCTTCTCCCGGACTCCGGGTAAATGA

SEQ ID NO: 2 - protein, 717 aa, Artificial Sequence
  21E10 (mouse anti-Human)
ATGAAGCTGCCTGTTCTGCTAGTGGTGCTGCTATTGTTCACGAGTCCAGCCTCAAGCAGTGATGTTGTTCTGACCCAAACTCCACTCTCTCTGCCTGTCAATATTGGAGATCAAGCCTCTATCTCTTGCAAGTCTACTAAGAGTCTTCTGAATAGTGATGGATTCACTTATTTGGACTGGTACCTGCAGAAGCCAGGCCAGTCTCCACAGCTCCTAATATATTTGGTTTCTAATCGATTTTCTGGAGTTCCAGACAGGTTCAGTGGCAGTGGGTCAGGAACAGATTTCACACTCAAGATCAGCAGAGTGGAGGCTGAGGATTTGGGAGTTTATTATTGCTTCCAGAGTAACTATCTTTACACGTTCGGAGGGGGGACCAAGCTGGAAATAAAACGGGCTGATGCTGCACCAACTGTATCCATCTTCCCACCATCCAGTGAGCAGTTAACATCTGGAGGTGCCTCAGTCGTGTGCTTCTTGAACAACTTCTACCCCAAAGACATCAATGTCAAGTGGAAGATTGATGGCAGTGAACGACAAAATGGCGTCCTGAACAGTTGGACTGATCAGGACAGCAAAGACAGCACCTACAGCATGAGCAGCACCCTCACGTTGACCAAGGACGAGTATGAACGACATAACAGCTATACCTGTGAGGCCACTCACAAGACATCAACTTCACCCATTGTCAAGAGCTTCAACAGGAATGAGTGTTAG

SEQ ID NO: 3 - protein, 1407 aa, Artificial Sequence
  29E3 (mouse anti-Human)
ATGGGATGGAGCTGGATCTTTCTCTTCCTCCTGTCAGGAACTGCAGGCGTCCACTCTGAGGTCCAGTTTCAGCAGTCAGGACCTGAGCTGGTGAAACCTGGGGCCTCAGTGAAGATATCCTGCAAGGCTTCTGGATACACATTCACTGACTACAACATGCACTGGGTGAAGCAGAGCCATGGAAAGAGCCTTGAGTGGATTGGATATATTTATCCTTACAATGGTGGGACTGGCTACAACCAGAAGTTCAAGAGCAAGGCCACATTGACTGTAGACAATTCCTCCAGAACAGCCTACATGGAACTCCGCAGCCTGTCATCTGAGGATTCTGCAGTCTATTACTGTGTAAGAAGGGATAGGTACGACGACCCGTTTGTTTACTGGGGCCAAGGGACTCTGGTCACTGTCTCTGCAGCCAAAACAACAGCCCCATCGGTCTATCCACTGGCCCCTGTGTGTGGAGATACAACTGGCTCCTCGGTGACTCTAGGATGCCTGGTCAAGGGTTATTTCCCTGAGCCAGTGACCTTGACCTGGAACTCTGGATCCCTGTCCAGTGGTGTGCACACCTTCCCAGCTGTCCTGCAGTCTGACCTCTACACCCTCAGCAGCTCAGTGACTGTAACCTCGAGCACCTGGCCCAGCCAGTCCATCACCTGCAATGTGGCCCACCCGGCAAGCAGCACCAAGGTGGACAAGAAAATTGAGCCCAGAGGGCCCACAATCAAGCCCTGTCCTCCATGCAAATGCCCAGCACCTAACCTCTTGGGTGGACCATCCGTCTTCATCTTCCCTCCAAAGATCAAGGATGTACTCATGATCTCCCTGAGCCCCATAGTCACATGTGTGGTGGTGGATGTGAGCGAGGATGACCCAGATGTCCAGATCAGCTGGTTTGTGAACAACGTGGAAGTACACACAGCTCAGACACAGACCCATAGAGAGGATTACAACAGTACTCTCCGGGTGGTCAGTGCCCTCCCCATCCAGCACCAGGACTGGATGAGTGGCAAGGAGTTCAAATGCAAGGTCAACAACAAAGACCTCCCAGCGCCCATCGAGAGAACCATCTCAAAACCCAAAGGGTCAGTAAGAGCTCCACAGGTATATGTCTTGCCTCCACCAGAAGAAGAGATGACTAAGAAACAGGTCACTCTGACCTGCATGGTCACAGACTTCATGCCTGAAGACATTTACGTGGAGTGGACCAACAACGGGAAAACAGAGCTAAACTACAAGAACACTGAACCAGTCCTGGACTCTGATGGTTCTTACTTCATGTACAGCAAGCTGAGAGTGGAAAAGAAGAACTGGGTGGAAAGAAATAGCTACTCCTGTTCAGTGGTCCACGAGGGTCTGCACAATCACCACACGACTAAGAGCTTCTCCCGGACTCCGGGTAAATGA

SEQ ID NO: 4 - protein, 723 aa, Artificial Sequence
  29E3 (mouse anti-Human)
ATGAAGCTGCCTGTTCTGCTAGTGGTGCTGCTATTGTTCACGAGTCCAGCCTCAAGCAGTGACATTGTGATGTCACAGTCTCCATCCTCCCTAGCTGTGTCAGTTGGAGAGAAGGTTACTATGAGCTGCAAGTCCAGTCAGAGCCTTTTATATAGTAGCAATCAAAAGAACTACTTGGCCTGGTACCAGCAGAAACCAGGGCAGTCTCCTAAACTGCTGATTTACTGGGCATCCACTAGGGAATCTGGGGTCCCTGATCGCTTCACAGGCAGTGGATCTGGGACAGATTTCACTCTCACCATCAGCAGTGTGAAGGCTGAAGACCTGGCAGTTTATTTCTGTCAGCAATATTATGGCTTTCCATTCACGTTCGGCTCGGGGACAAAGTTGGAAATAAAGCGGGCTGATGCTGCACCAACTGTATCCATCTTCCCACCATCCAGTGAGCAGTTAACATCTGGAGGTGCCTCAGTCGTGTGCTTCTTGAACAACTTCTACCCCAAAGACATCAATGTCAAGTGGAAGATTGATGGCAGTGAACGACAAAATGGCGTCCTGAACAGTTGGACTGATCAGGACAGCAAAGACAGCACCTACAGCATGAGCAGCACCCTCACGTTGACCAAGGACGAGTATGAACGACATAACAGCTATACCTGTGAGGCCACTCACAAGACATCAACTTCACCCATTGTCAAGAGCTTCAACAGGAATGAGTGTTAG

SEQ ID NO: 5 - protein, 1404 aa, Artificial Sequence
  178 (rat-anti-mouse)
ATGAAATGCAGCTGGATCAACCTCTTCTTGATGGCACTAGCTTCAGGGGTCTACGCAGAAGTACAGCTGCAGCAGTCTGGGCCCGAGCTTCGGAGACCTGGGTCCTCAGTCAAGTTGTCTTGTAAGGCTTCTGGCTACAGTATTATAGATTACCTTATGCACTGGGTAAAACATAGGCCAGGACACGGCCTGGAATGGATAGGATGGATTGATCCTGAAAATGGTGAAACAAAATATGCTCAGAAGTTCCAAAGGAAGGCCACACTGACTGCAGATACATCCTCCAGCACAGCCTACATGCAACTCAGCAGCCTGACGTCTGAGGACACAGCAACCTATTTTTGTGCTAGACAACAACTACGGTATGTTATGGATGCCTGGGGTCAAGGAGGTTCAGTCACTGTCTCCTCAGCCAAAACAACAGCCCCATCGGTCTATCCACTGGCCCCTGTGTGTGGAGATACAACTGGCTCCTCGGTGACTCTAGGATGCCTGGTCAAGGGTTATTTCCCTGAGCCAGTGACCTTGACCTGGAACTCTGGATCCCTGTCCAGTGGTGTGCACACCTTCCCAGCTGTCCTGCAGTCTGACCTCTACACCCTCAGCAGCTCAGTGACTGTAACCTCGAGCACCTGGCCCAGCCAGTCCATCACCTGCAATGTGGCCCACCCGGCAAGCAGCACCAAGGTGGACAAGAAAATTGAGCCCAGAGGGCCCACAATCAAGCCCTGTCCTCCATGCAAATGCCCAGCACCTAACCTCTTGGGTGGACCATCCGTCTTCATCTTCCCTCCAAAGATCAAGGATGTACTCATGATCTCCCTGAGCCCCATAGTCACATGTGTGGTGGTGGATGTGAGCGAGGATGACCCAGATGTCCAGATCAGCTGGTTTGTGAACAACGTGGAAGTACACACAGCTCAGACACAGACCCATAGAGAGGATTACAACAGTACTCTCCGGGTGGTCAGTGCCCTCCCCATCCAGCACCAGGACTGGATGAGTGGCAAGGAGTTCAAATGCAAGGTCAACAACAAAGACCTCCCAGCGCCCATCGAGAGAACCATCTCAAAACCCAAAGGGTCAGTAAGAGCTCCACAGGTATATGTCTTGCCTCCACCAGAAGAAGAGATGACTAAGAAACAGGTCACTCTGACCTGCATGGTCACAGACTTCATGCCTGAAGACATTTACGTGGAGTGGACCAACAACGGGAAAACAGAGCTAAACTACAAGAACACTGAACCAGTCCTGGACTCTGATGGTTCTTACTTCATGTACAGCAAGCTGAGAGTGGAAAAGAAGAACTGGGTGGAAAGAAATAGCTACTCCTGTTCAGTGGTCCACGAGGGTCTGCACAATCACCACACGACTAAGAGCTTCTCCCGGACTCCGGGTAAATGA

SEQ ID NO: 6 - protein, 726 aa, Artificial Sequence
  178 (rat-anti-mouse)
ATGTCAGGACACAATTTAGATATGAGGGTCCAGATTCAGTTTTGGGGGCTTCTTCTGCTCTGGACATCAGGTATACAGTGTGATGTCCAGATGACCCAGTCTCCATATAATCTTGCTGCCTCTCCTGGAGAAAGTGTTTCCATCAATTGCAAGGCAAGTAAGAGCATTAGCAAGTATTTAGCCTGGTATCAACAGAAACCTGGGAAAACAAATAAGCTTCTTATCTACGATGGGTCAACTTTGCAATCTGGAATTCCATCGAGGTTCAGTGGCAGTGGATCTGGTACAGATTTCACTCTCACCATCAGAAGCCTGGAGCCTGAAGATTTTGGGCTCTATTACTGTCAACAGCATAATGAATACCCGCTCACGTTCGGTTCTGGGACCAAGCTGGAGATCAAACGGGCTGATGCTGCACCAACTGTATCTATCTTCCCACCATCCACGGAACAGTTAGCAACTGGAGGTGCCTCAGTCGTGTGCCTCATGAACAACTTCTATCCCAGAGACATCAGTGTCAAGTGGAAGATTGATGGCACTGAACGACGAGATGGTGTCCTGGACAGTGTTACTGATCAGGACAGCAAAGACAGCACGTACAGCATGAGCAGCACCCTCTCGTTGTCCAAGGCTGACTATGAAAGTCATAACCTCTATACCTGTGAGGTTGTTCATAAGACATCATCCTCACCCGTCGTCAAGAGCTTCAACAGGAATGAGTGTTAG

(19) United States
(12) Patent Application Publication
(10) Pub. No.: US 2023/0183340 A1
(43) Pub. Date: Jun. 15, 2023

(54) Compositions and Methods for Reducing Resistance to or Enhancing Immunotherapy

(71) Applicant: Washington University, St. Louis, MO (US)

(72) Inventor: Marco Colonna, St. Louis, MO (US)

(73) Assignee: Washington University, St. Louis, MO (US)

(21) Appl. No.: 17/802,631

(22) PCT Filed: Feb. 26, 2021
(86) PCT No.: PCT/US2021/019914

§ 371 Date: Aug. 26, 2022

Related U.S. Application Data
(60) Provisional application No. 63/036,121, filed on Jun. 8, 2020.
(60) Provisional application No. 62/981,827, filed on Feb. 26, 2020.

(51) Int. Cl. C07K 16/28; A61P 35/00

(52) U.S. Cl. CPC C07K 16/2803; A61P 35/00; C07K 16/2818; A61K 2039/505

(57) ABSTRACT

Among the various aspects of the present disclosure is the provision of methods and compositions for reducing checkpoint immunotherapy resistance or enhance checkpoint immunotherapy efficacy comprising treatment with a TREM2 inhibiting agent having reduced Fc effector function.

### CROSS-REFERENCE TO RELATED APPLICATIONS

This application claims priority from U.S. Provisional Application Serial No. 62/981,827 filed on 26 Feb. 2020 and U.S. Provisional Application Serial No. 63/036,121 filed on 08 Jun. 2020, which are incorporated herein by reference in their entireties.

### STATEMENT REGARDING FEDERALLY SPONSORED RESEARCH OR DEVELOPMENT

Not applicable.

### MATERIAL INCORPORATED-BY-REFERENCE

The Sequence Listing, which is a part of the present disclosure, includes a computer-readable form comprising nucleotide and/or amino acid sequences of the present invention. The subject matter of the Sequence Listing is incorporated herein by reference in its entirety.

The present disclosure generally relates to enhancing immunotherapies, such as checkpoint inhibitor therapies.

### SUMMARY

Among the various aspects of the present disclosure is the provision of methods and compositions for reducing checkpoint immunotherapy resistance or enhancing checkpoint immunotherapy efficacy.

An aspect of the present disclosure provides for a method of suppressing tumor growth in a subject having cancer, comprising: administering a TREM2 inhibiting agent and/or an immunotherapy to a subject in an amount effective to suppress tumor growth, wherein the TREM2 inhibiting agent has TREM2 inhibiting function; the TREM2 inhibiting agent comprises an antibody or a functional fragment or variant thereof; or the TREM2 inhibiting agent results in a reduction or loss of effector function. Another aspect of the present disclosure provides for a method of reducing myeloid-induced immune cell suppression comprising blocking TREM2 on a myeloid cell comprising contacting a TREM2 inhibiting agent to the myeloid cell, wherein the TREM2 inhibiting agent has TREM2 binding activity or TREM2 blocking function and/or a reduction or loss of effector function. Yet another aspect of the present disclosure provides for a method of blocking TREM2 function and/or recruiting immune cells in a subject or cells comprising administering a TREM2 inhibiting agent to the subject or the cells of the subject receiving, having received, or to receive immunotherapy. Yet another aspect of the present disclosure provides for a method of reducing checkpoint immunotherapy resistance in a subject or the cells of a subject in need thereof comprising administering a TREM2 inhibiting agent to the subject receiving, having received, or to receive immunotherapy. Yet another aspect of the present disclosure provides for a method of enhancing immunotherapy efficacy in a subject receiving, having received, or to receive immunotherapy comprising administering a TREM2 inhibiting agent to the subject or the cells of a subject, wherein the TREM2 inhibiting agent modifies tumor-infiltrating myeloid cells to maintain an environment hospitable to immune cells. Yet another aspect of the present disclosure provides for a method of reshaping or remodeling of intratumoral, tumor-associated macrophage infiltrate population, comprising administering a TREM2 inhibiting agent and/or immunotherapy to a subject. Yet another aspect of the present disclosure provides for a method of treating a subject, wherein the subject has cancer or is suspected of having cancer comprising: measuring an amount of TREM2 in a sample; and/or if TREM2 is elevated compared to control, the subject is predicted to have a poor prognosis; or if TREM2 is elevated compared to control, the subject is treated with a TREM2 inhibiting agent and/or immunotherapy. In some embodiments, the TREM2 inhibiting agent comprises a mutant or dysfunctional Fc region or Fc domain, resulting in a loss or reduced effector function. In some embodiments, the TREM2 inhibiting agent comprises a loss of function mutation in the Fc region or Fc domain to result in a reduction or loss of Fc effector function. In some embodiments, the TREM2 inhibiting agent comprises an Fc mutation comprising an amino acid addition, insertion, deletion, substitution, or combination thereof. In some embodiments, the TREM2 inhibiting agent comprises an addition of one or more glycans in an Fc domain. In some embodiments, the TREM2 inhibiting agent comprises a variable region of a heavy chain grafted onto a constant region backbone mutated in an Fc domain. In some embodiments, the TREM2 inhibiting agent comprises LALAPG mutations that abrogate Fc effector functions. In some embodiments, the TREM2 inhibiting agent comprises an anti-TREM2 antibody construct, Fc-fusion antibody-like protein, an anti-TREM2 antibody, recombinant anti-TREM2 antibody or protein, a functional portion or fragment thereof, a fusion protein, scFv, peptide, diabody, unibody, or a functional fragment, variant, or mutant, thereof, or small molecule having TREM2 inhibiting or blocking function. In some embodiments, the TREM2 inhibiting agent comprises an anti-TREM2 mAb. In some embodiments, the TREM2 inhibiting agent comprises an Fc-mutated anti-TREM2 monoclonal antibody (mAb). In some embodiments, the TREM2 inhibiting agent is a recombinant form of an anti-TREM2 mAb. In some embodiments, the TREM2 inhibiting agent comprises 21 E10 mAb or mAb 178, or functional fragment or variant thereof, specific for human TREM2. In some embodiments, the TREM2 inhibiting agent comprises a mutation comprising an amino acid addition, insertion, deletion, substitution, or combination thereof. In some embodiments, the TREM2 inhibiting agent comprises an Fc mutation, wherein the Fc mutation prevents or reduces effector function; recognition by Fc receptors; recognition by complement or depletes antibody fix complement; induces antibody-dependent cellular cytotoxicity; or antibody-dependent phagocytosis. In some embodiments, the TREM2 inhibiting agent targets, inhibits, prevents, reduces, or blocks TREM2 function. In some embodiments, the TREM2 inhibiting agent modifies tumor-infiltrating myeloid cells to maintain an environment hospitable to immune cells, optionally, T-cells. In some embodiments, the TREM2 inhibiting agent has TREM2 blocking function and/or a loss of Fc effector function. In some embodiments, the TREM2 inhibiting agent recruits T-cells. In some embodiments, an effective amount or a therapeutically effective amount of the TREM2 inhibiting agent is an amount sufficient to induce immunostimulatory macrophages or reduce immunosuppressive macrophages. In some embodiments, the amount or a therapeutically effective amount of the TREM2 inhibiting agent is an amount sufficient to make tumor microenvironment hospitable to T-cells; recruit T-cells to a tumor microenvironment; have loss or reduction of effector function; maintain immune system; or recruit immune cells. In some embodiments, the amount or a therapeutically effective amount of the TREM2 inhibiting agent results in enhanced immunostimulation; prevention of cytokine storm in checkpoint blockade therapy; prevention of cytokine release syndrome (e.g., in a subject receiving CAR-T therapy); reduced checkpoint immunotherapy resistance; improving T-cell response; or enhanced checkpoint immunotherapy efficacy compared to a subject prior to receiving or a subject not receiving a TREM2 inhibiting agent therapy. In some embodiments, the amount or a therapeutically effective amount of the TREM2 inhibiting agent prevents, targets, inhibits, blocks, or reduces TREM2 function, signaling, or activity, but does not kill or substantially deplete or kill macrophages or myeloid cells. In some embodiments, macrophages are depleted compared to macrophages contacted with TREM2 inhibiting agent having a functional Fc (e.g., between about 10% and about 20% depletion). In some embodiments, the TREM2 inhibiting agent does not cause antibody-dependent cellular cytotoxicity (ADCC) or antibody-dependent phagocytosis (ADP) and/or inhibits TREM2-ligand interaction. In some embodiments, the TREM2 inhibiting agent prevents, blocks, or reduces TREM2 function, signaling, or activity and/or does not substantially kill myeloid or macrophages or reduce an amount of myeloid or macrophage cells compared to a TREM2 inhibiting agent having Fc effector function. In some embodiments, the TREM2 inhibiting agent induces a skewing or increase in the ratio of immunostimulating myeloid cells to immunosuppressive myeloid cells in the myeloid compartment and/or promotes T cell activation. In some embodiments, MRC1 (CD206) is a correlative marker of immunosuppressive activity; and/or iNOS (NOS2) is a correlative marker of immunostimulatory activity. In some embodiments, the administering the TREM2 inhibiting agent results in reduced Ly6Chi myeloid cells and/or increased CD8+ and/or PD1+ T cells; or increased IFNλ-producing CD8+ T cells and/or TNFα-producing CD4+ T cells. In some embodiments, the TREM2 inhibiting agent changes macrophage populations infiltrating a tumor or wherein CX3CR1+ and MRC1+ macrophage subsets declined or subsets expressing potentially immunostimulatory molecules were induced. In some embodiments, the immunotherapy is selected from a checkpoint immunotherapy or CAR-T therapy. In some embodiments, the checkpoint immunotherapy is a checkpoint blockade therapy. In some embodiments, the checkpoint immunotherapy is checkpoint inhibitor therapy selected from anti-PD-1. In some embodiments, the immunotherapy is selected from CAR-T. In some embodiments, the TREM2 inhibiting agent targets immunosuppressive myeloid cells (e.g., M2-like macrophages, myeloid progenitor cells, or immature myeloid cells collectively defined as myeloid-derived suppressor cells (MDSCs)). In some embodiments, the immune cell is an immunosuppressive myeloid cell or an immunostimulatory myeloid cell. In some embodiments, the immune cell is an immunostimulatory myeloid cell selected from type 1 dendritic cells (DC1s) or M1-like IFN-y-induced macrophages. In some embodiments, the cancer is associated with a microenvironment infiltrated by macrophages expressing TREM2. In some embodiments, the cancer is selected from sarcoma, progressors, colorectal carcinoma (CRC), breast cancer, or triple-negative breast cancer (TNBC). In some embodiments, the TREM2 inhibiting agent is a TREM2 neutralizing antibody or a functional fragment or variant thereof expressed on a cell. In some embodiments, a TCR is ectopically expressed on a cell. In some embodiments, the TREM2 inhibiting agent is a TREM2 neutralizing antibody or a functional fragment or variant thereof expressed on a CAR-T cell. In some embodiments, TREM2 expression is associated with a reduced overall survival or relapse free survival in CRC or TNBC.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. TREM2 is a pro-tumorigenic marker of tumor-infiltrating macrophages in mouse models and human tumors that can be targeted to curb tumor growth and improve the efficacy of checkpoint blockade therapy while remodeling the landscape of tumor-infiltrating macrophages.

FIG. 2. TREM2 Deficiency Attenuates Growth of Transplantable Tumors. (A and B) Tumor growth in Trem2^{+/+} and Trem2^{-/-} mice injected subcutaneously (s.c.) with MCA/1956. (C) Flow cytometry analysis of the immune infiltrate of MCA/1956 tumors 10 days after injection. (D) Representative flow plots of PD-1 expression in CD8⁺ (left panels) and CD4⁺ (right panels) T cells. (E) Tumor growth of MCA/1956 in Trem2^{+/+} and Trem2^{-/-} after depletion of CD8⁺ T cells. Growth curves of groups (A and E) and single mice (B) are shown. Significance at day 20 (E) are as follows: **: Trem2^{+/+} versus Trem2^{-/-}; ***: Trem2^{-/-} versus Trem2^{+/+} αCD8; ***: Trem2^{-/-} versus Trem2^{-/-} αCD8; ns: Trem2^{+/+} versus Trem2^{+/+} αCD8; ns: Trem2^{+/+} versus Trem2^{-/-} αCD8; ns: Trem2^{+/+} αCD8 versus Trem2^{-/-} αCD8. Data represent mean ± SEM. *p ≤ 0.05, **p ≤ 0.01, ***p ≤ 0.001. Two-way Anova with multiple comparisons (A and E) and Mann-Whitney test (C). See also FIG. 3.

FIG. 3. TREM2 Deficiency Attenuates Growth of Transplantable Tumors, Related to FIG. 2. (A and B) Flow cytometry analysis of the immune infiltrate of MCA/1956, 10 days after injection. The myeloid compartment (A) and lymphoid cells (B) are shown. (C) Tumor growth of MCA/1956 in Trem2^{+/+} and Trem2^{-/-} after depletion of CD4 T cells. CD8 T cell depletion from the same experiment is shown in FIG. 2E; controls are shown in both. (D and E) Tumor growth in Trem2^{+/+} and Trem2^{-/-} mice injected s.c. with the MC38 cell line. (F) Flow cytometry analysis of the immune infiltrate of MC38 tumors, 10 days after injection. Representative flow plots of CD206⁺ macrophages in Trem2^{+/+} and Trem2^{-/-} mice are shown. (G and H) Growth of PyMT mammary tumor in Trem2^{+/+} and Trem2^{-/-} mice injected intramammary (i.m.). Growth curves of groups (C, D, G) and single mice (E, H) are shown. Data represent mean ± SEM. *p ≤ 0.05, **p ≤ 0.01, ***p ≤ 0.001; Two-way Anova and Mann Whitney test.

FIG. 4. TREM2 Deficiency Remodels the Myeloid Compartment in MCA-Derived Sarcoma, Related to FIG. 5. (A) UMAP plots of intratumoral immune populations from merged conditions. (B) Cluster proportions in each condition: Trem2^{+/+} and Trem2^{-/-}. Cluster identities were based on expression of key markers shown below. (C) UMAP plots of clusters in each condition. (D) UMAP plots of selected cluster markers from merged conditions. (E) Violin plots of TREM2 expression level in each cluster. (F) UMAP plots of re-clustered lymphoid cells from merged conditions. (G) Cluster proportions in each condition: Trem2^{+/+} and Trem2^{-/-}. Cluster identities were based on expression of key markers shown below. (H) UMAP plots of clusters in each condition. (I) UMAP plots of selected genes (Ifng and Pdcd1) in lymphoid clusters in each condition.

FIG. 5. TREM2 Deficiency Remodels the Myeloid Compartment in MCA-Derived Sarcoma. (A) UMAP plots of macrophage clusters from merged conditions. (B) Cluster proportions in each condition: Trem2^{+/+} and Trem2^{-/-}. Cluster identities were based on expression of key markers shown below. (C) UMAP plots of selected cluster markers from merged conditions. (D) UMAP plots of macrophage clusters in each condition. (E) Violin plots of TREM2 expression level in each cluster. (F) TREM2 expression in each condition. (G) Heatmap showing normalized expression of selected genes in each macrophage cluster for each condition. Tumors from Trem2^{+/+} and Trem2^{-/-} male mice were analyzed 10 days after injection. See also FIG. 4.

FIG. 6. TREM2 Deficiency Drives a Complex Remodeling of Macrophage Infiltrate that Partly Resembles that Observed Following ICT. Feature plots of selected genes in MCA/1956 sarcoma in Trem2^{+/+} and Trem2^{-/-} mice (left panels) and in T3 sarcoma (Gubin et al., 2018) following ICT.

FIG. 7. TREM2 Deficiency Enhances Anti-PD-1 Immunotherapy. (A) Experimental setup of the anti-PD-1 treatment. Optimal treatment started at day 3 (αPD-1-I); suboptimal treatment started at day 8 (αPD-1-II). (B-E) Tumor growth of MCA/1956 (B and C) and MC38 (D and E) in Trem2^{+/+} and Trem2^{-/-} mice treated with anti-PD-1. (B) Significance at day 20: ***: Trem2^{+/+} αPD-1 versus Trem2^{-/-} αPD-1. (D) Significance at day 20: ***: Trem2^{+/+} αPD-1 versus Trem2^{-/-} αPD-1. (F) Flow cytometry analysis of lymphoid cells in Trem2^{+/+} and Trem2^{-/-} mice treated with anti-PD-1 (day 14 after tumor injection). Growth curves of groups (A, B, and D) and single mice (C and E) are shown. Data represent mean ± SEM. *p ≤ 0.05, **p ≤ 0.01, ***p ≤ 0.001; Two-way Anova with multiple comparisons (B and D) and Mann-Whitney test (F) were performed.

FIG. 8. TREM2 Engagement Reduces MCA-Sarcoma Growth and Remodels the Tumor Microenvironment, Related to FIG. 9 and FIG. 10. (A) Histograms (left) and curves of mean fluorescence intensity (MFI) (right) of TREM2 staining at indicated antibody concentrations. Native anti-murine TREM2 (clone 178) and Fc-mutated anti-murine TREM2 (clone m178) were used. (B) GFP expression in TREM2 reporter cell line after stimulation with HDL in the presence or absence of controls and anti-TREM2 antibodies. Native (178) and Fc-mutated anti-TREM2 (m178) were used. Fc-mutated anti-human ILT1 (m135.5) and medium only (ctrl) were used as controls. (C) TREM2 expression in peritoneal myeloid cells after treatment with m178 mAb. Mice treated with anti-human ILT1 (m135.5) and Trem2^{-/-} mice were used as controls. TREM2⁺ macrophages were detected using an anti-TREM2 antibody that does not interfere with mAb 178 binding to TREM2. (D) Flow cytometry analysis of the myeloid immune compartment 10 days after tumor injection in mice treated with anti-TREM2. Control groups, mice treated with anti-TREM2, anti-PD-1, and the combination of anti-TREM2 and anti-PD-1 are shown. (E) Flow cytometry analysis of myeloid cells 24 days after tumor injection in mice treated with anti-TREM2. Control groups, mice treated with anti-TREM2 and anti-PD-1 are shown. Mice treated with the combination of anti-TREM2 and anti-PD-1 rejected the tumor (ND). (F) Confocal images of MCA/1956 sarcoma 24 days after injection of tumor cells in mice treated with anti-TREM2. Representative pictures of CD206 and Iba1 expression within the tumor and CD206/Iba1 ratio are shown. (G) UMAP plots of intratumoral immune populations from merged conditions. (H) Cluster proportions in each condition: controls (CTRL); mice treated with anti-PD-1 (αPD-1); mice treated with anti-TREM2 (αTREM2); mice treated with the combination of anti-PD-1 and anti-TREM2 (αTREM2 + αPD-1). Cluster identities are based on expression of key markers (shown below). (I) UMAP plots of clusters in each condition. (J) UMAP plots of selected cluster markers from merged conditions. Data represent mean ± SEM. *p ≤ 0.05, **p ≤ 0.01, ***p ≤ 0.001; Mann Whitney test.

FIG. 9. TREM2 Engagement Reduces MCA-Sarcoma Growth and Enhances Anti-PD-1-Induced Tumor Regression. (A and B) Tumor growth in mice injected s.c. with the MCA/1956 cell line treated with anti-TREM2 and anti-PD-1 antibodies. (A) Significance at day 24: ***: αPD-1 versus αTREM2+αPD-1. (C) Tumor growth of MCA/1956 tumors in Trem2^{+/+} and Trem2^{-/-} mice treated with anti-TREM2. Anti-ILT1 was used as a control in both Trem2^{+/+} and Trem2^{-/-} mice. Significance at day 20: **: CTRL versus CTRL Trem2^{-/-}; ***: CTRL versus αTREM2; ns: CTRL Trem2^{-/-} versus αTREM2 Trem2^{-/-}. (D) Flow cytometry analysis of the myeloid immune compartment 10 days after tumor injection in mice treated with anti-TREM2. Control groups, mice treated with anti-TREM2, anti-PD-1, and the combination of anti-TREM2 and anti-PD-1 are shown. (E) IFNγ and TNFα production by CD8 and CD4 T cells stimulated ex vivo with PMA/I. Data represent mean ± SEM. *p ≤ 0.05, **p ≤ 0.01, ***p ≤ 0.001; Two-way Anova with multiple comparisons (A and C) and Mann-Whitney test (D and E) were performed. Growth curves of groups (A and C) and single mice (B) are shown. See also FIG. 8.

FIG. 10. Anti-TREM2 Treatment Remodels the Myeloid Compartment in MCA-Derived Sarcoma. (A) UMAP plots of macrophage clusters from merged conditions. (B) Cluster proportions in each condition: controls (CTRL); mice treated with anti-PD-1 (αPD-1); mice treated with anti-TREM2 (αTREM2); mice treated with the combination of anti-PD-1 and anti-TREM2 (αTREM2 + αPD-1). Cluster identities were based on expression of key markers shown below. (C) TREM2 expression in macrophage clusters from merged conditions. (D) UMAP plots of selected cluster markers from merged conditions. (E) UMAP plots of myeloid clusters in each condition. (F) Heatmap showing normalized expression of selected genes in each macrophage cluster for each condition. Tumors from control and treated female mice were analyzed 10 days after injection. See also FIG. 8 and FIG. 11.

FIG. 11. TREM2 Engagement Remodels the Myeloid Compartment in MCA Tumors, Related to FIG. 10. (A) UMAP plots of selected genes marking cluster 6 (C3, Cd38 and Ly6i) and cluster 5 (∥1r2, Cd244, Spp1). (B) Enrichment of distinct gene signatures in clusters 5 and 6. Clusters from macrophage reclustering and merged conditions are shown (A and B). (C) Feature plots of selected genes in each condition. (D) Heatmap showing the comparison of gene signatures of clusters from tumor bearing Trem2^{+/+} and Trem2^{-/-} mice (columns) and anti-TREM2-treated mice (rows).

FIG. 11. TREM2 Engagement Remodels the Myeloid Compartment in MCA Tumors, Related to FIG. 10. (A) UMAP plots of selected genes marking cluster 6 (C3, Cd38 and Ly6i) and cluster 5 (∥1r2, Cd244, Spp1). (B) Enrichment of distinct gene signatures in clusters 5 and 6. Clusters from macrophage reclustering and merged conditions are shown (A and B). (C) Feature plots of selected genes in each condition. (D) Heatmap showing the comparison of gene signatures of clusters from tumor bearing Trem2^{+/+} and Trem2^{-/-} mice (columns) and anti-TREM2-treated mice (rows).

FIG. 12. TREM2 Protein Expression in Human Non-neoplastic and Neoplastic Tissues, Related to FIG. 13. (A) IHC of tissue sections stained for anti-TREM2. Sections derived from normal human skin (a), lung (b), liver (c), brain (d), colon (e), stomach (f), uterus (g), and placenta (h). TREM2 is expressed in alveolar macrophages, microglia, macrophages of the endometrial stroma and placental macrophages. No expression is found in the majority of tissue macrophages in liver, skin, and gut. Sections are counterstained with hematoxylin. Magnification: 200×, scale bar: 100 µm. (B) TREM2⁺ tumor macrophages display variable morphology, including small monocytoid cells (a), multinucleated giant forms (b) and foamy cells (c). No co-localization is observed between TREM2 and DC markers including BDCA2 (d), CD1c (e) and CD207 (f). Sections are counterstained with hematoxylin. Magnification: 600× (a-c; scale bar: 33 µm) and 200× (d-f; scale bar: 100 µm). (C) Localization of TREM2⁺ tumor macrophages. Sections from human melanoma (a) and lung carcinomas (b) are double stained for CD163 (blue) and TREM2 (brown); TREM2⁺CD163⁺ tumor macrophages are restricted to the intra-tumor area. Low grade papillary urothelial carcinoma (c) benign nevi (e), and colon adenoma (g) are poorly infiltrated by TREM2⁺ macrophages compared to their associated malignant counterpart (respectively d, f and h). Sections are counterstained with hematoxylin. Magnification: 100× (a, b; scale bar: 200 µm) and 200× (c-h; scale bar: 100 µm).

FIG. 13. TREM2 Expression in Human Cancers and Association with Prognosis in CRC and TNBC Patients. (A) IHC of tissue sections immunostained with anti-human TREM2 from primary carcinomas of skin (a), liver (b), lung (c), breast (d), bladder (e), colon (f), stomach (g), pancreas (h), and kidney (i), as well as from nodal lymphoma (j), cutaneous melanoma (k), and brain glioma (I). Sections are counterstained with hematoxylin. Magnification: 200×, scale bar: 100 µm. (B) Morphology and phenotype of TREM2⁺ tumor macrophages. Sections are from primary carcinomas and melanomas and stained as labeled. TREM2 reactivity decorates the cell membrane as observed in high-power view (a). TREM2⁺ tumor macrophages co-stain for CD163 (b), CD68 (c), nuclear MAFB (d), CSF1R (e), and the MITF transcription factor (f). Sections are counterstained with hematoxylin. Magnification: 600× (a, scale bar: 33 µm) and 200× (b-f, scale bar: 100 µm). (C and D) Kaplan-Meier survival curves generated for TREM2 expression. Patients were divided in high- and low-expressing groups based on 75% quantile of TREM2 expression. TCGA CRC (C) and TNBC (D) cohorts are shown. See also FIG. 12, FIG. 14, and FIG. 15.

FIG. 14. TREM2⁺ Tumor Macrophages Localize to Regional and Distant Metastasis, Related to FIG. 13. IHC of tissue sections stained from TREM2. Sections derive from liver (a, b), lung (c) and lymph nodes (d-f) metastasis of melanomas (a, c), ovarian serous carcinoma (b) breast carcinoma (d), colorectal (e) and lung adenocarcinoma (f). TREM2+ tumor macrophages are selectively found within the metastatic nodules, whereas the normal surrounding tissue is spared. Sections are counterstained with hematoxilin Magnification: 100×, scale bar: 200 µm.

FIG. 15. TREM2 Expression Correlates with a Gene Signature of Tumor Macrophages in TCGA Cohorts, Related to FIG. 13. (A and B) Correlation data for TREM2 (y axis) and the indicated genes in each cohort (x axis). Individual scatterplots of the top 15 genes correlating with TREM2 are shown.

FIG. 16. TREM2-deficient mice are less susceptible to transplantable models of MCA-derived sarcoma, MC38 colon carcinoma and PyMT breast cancer.

FIG. 17. TREM2-deficiency is associated with reduced Ly6Chi myeloid cells and increased CD8+ and PD1+ T cells.

FIG. 18. TREM2-deficient mice are less susceptible to transplantable models of MCA-derived sarcoma, MC38 colon carcinoma and PyMT breast cancer.

FIG. 19. Anti-PD-1 treatment protocol. aPD1: i.p. injection at days 3, 6, 9, 12, 15 (200 µg/mouse). aPD1II: i.p. injection at days 8, 11, 14, 17 (200 µg/mouse) - suboptimal protocol.

FIG. 20. TREM2-deficiency enhances anti-PD-1 response in MCA sarcoma.

FIG. 21. TREM2-deficiency enhances anti-PD-1 response in MC38 colon carcinoma.

FIG. 22. Anti-TREM2-treatment is protective and enhances anti-PD-1 response in MCA sarcoma. Recombinant Fc mutated anti-TREM2 (clone 178-IgG2a), injected i.p. (200 µg/mouse) at days 2, 7, 12, 17. Recombinant Fc mutated anti-ILT1 (human, not expressed in the mouse) is used as a control. Exp I.

FIG. 23. Anti-TREM2-treatment is protective and enhances anti-PD-1 response in MCA sarcoma. Recombinant Fc mutated anti-TREM2 (clone 178-IgG2a), injected i.p. (200 µg/mouse) at days 2, 7, 12, 17. Recombinant Fc mutated anti-ILT1 (human, not expressed in the mouse) is used as a control. Exp II.

FIG. 24. Anti-TREM2-treatment remodels the myeloid compartment.

FIG. 25. Targeting of human TREM2 is protective in the MCA1956 sarcoma model. Mean tumor diameter of MCA1956 tumors injected in TREM2CV mice and treated with a control antibody or with the anti-hTREM2 mAb (21E10). *p ≤ 0.05. Mann Whitney test was performed.

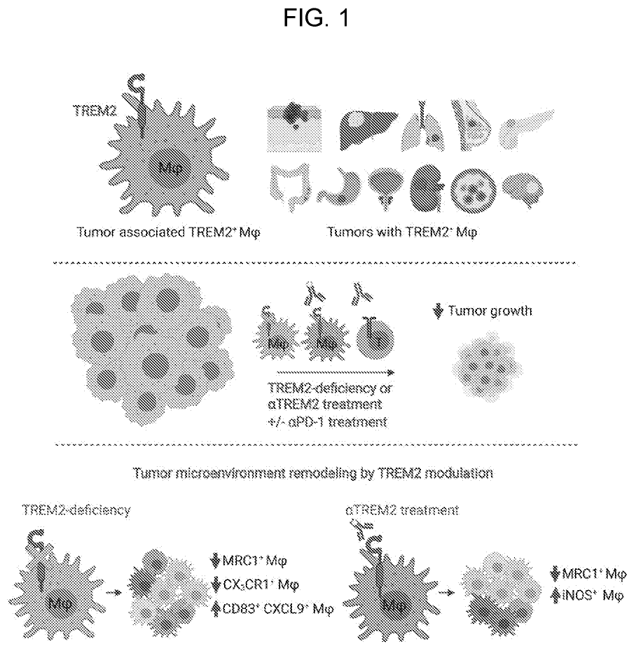

### DETAILED DESCRIPTION OF THE INVENTION

The present disclosure is based, at least in part, on the discovery that inhibiting or blocking TREM2 can reduce checkpoint immunotherapy resistance or enhance checkpoint immunotherapy efficacy.

Shown herein is a TREM2 blocking antibody that targets myeloid tumor cells to enhance checkpoint inhibitor therapies, such as those directed against PD-1.

TREM2 was shown to be involved in promoting tumorigenesis by altering the composition of the tumor microenvironment in a manner that benefits the tumor, specifically by alternating the composition of innate immune cells, thereby enhancing immune evasion.

Here is shown the validation of the technology both genetically and using a functionally blocking antibody combined with suboptimal PD-1 antibody treatment.

In one embodiment, is a combination treatment that may overcome additional immune evasion mechanisms (e.g., beyond T cells) to facilitate the wider application of checkpoint inhibitor therapy.

As shown herein:
1. Trem2^{-/-} mice were more resistant to tumor growth than wild type (WT) mice in models of MCA- induced sarcoma, colorectal cancer, and mammary tumor.
2. Phenotypic analysis of Trem2^{-/-} mice showed alterations in macrophage subsets and an increase of CD8+ T cells, some of which expressed PD-1.
3. PD-1 immunotherapy of MCA sarcoma was more effective in Trem2^{-/-} than WT mice.
4. Antibody blockade of TREM2 curbed tumor growth and led to complete tumor regression when performed in association with suboptimal PD-1 immunotherapy.
5. Analysis of the myeloid cell landscape by scRNA-seq showed that anti-TREM2 mAb remodeled tumor infiltrating macrophages: a novel subset with immunomodulatory features was induced; in parallel, other immunosuppressive cell clusters proportionately declined.

Also described herein is the extensive examination of human clinical and pathological data, demonstrating that:
1. TREM2 is a marker of infiltrating macrophages in over 200 human tumors examined by immunohistochemistry. Moreover, TREM2 was the only macrophage marker exclusively expressed within the tumor, while other markers were expressed within and outside of the tumor.
2. TREM2 expression inversely correlated with greater overall survival and relapse free survival in colorectal carcinoma and triple-negative breast cancer.

Checkpoint immunotherapy unleashes T cell effector functions that control tumor growth, but can be undermined by myeloid cells that induce immunosuppression. TREM2 is a myeloid surface receptor that binds lipids and transmits intracellular signals through protein-tyrosine phosphorylation known to sustain microglial responses during Alzheimer’s disease. Intriguingly, TREM2 expression has recently been noted in tumor infiltrating macrophages. We found that Trem2^{-/-} mice are more resistant to growth of sarcoma, colorectal and mammary cancer cells than wild-type mice and are more responsive to anti-PD-1 immunotherapy. Furthermore, antibody blockade of TREM2 curbed tumor growth and led to complete tumor regression when combined with anti PD-1. scRNA-seq revealed that TREM2 blockade induced a novel subset of macrophages with immunostimulatory features, while those considered immunosuppressive declined. TREM2 expression was evident in tumor macrophages in over 200 human cancer cases examined and inversely correlated with prolonged survival for two types of cancer.

Thus, TREM2 is a promising target to modify tumor infiltrating myeloid cells and effectively augment checkpoint immunotherapy.

The present disclosure provides for the combination of a TREM2 blocking antibody or inhibitor in combination with checkpoint inhibitor therapy (e.g., anti-PD-1).

In summary, TREM2 is expressed in tumor-associated macrophages; TREM2 deficiency is associated with reduced growth and enhanced anti-PD-1 response in MCA-sarcoma and MC38-colon carcinoma; anti-TREM2 treatment is protective in MCA-sarcoma and boosts anti-PD-1 response in MCA-sarcoma; TREM2-deficiency is associated with a reduced infiltrate of Ly6Chigh myeloid cells; anti-TREM2 treatment induced a skewing in the myeloid compartment and promotes T cell activation; TREM2 expression is associated with a reduced overall survival and relapse free survival in CRC and TNBC; and TREM2 expression correlates with TAM genes in cancer patients.

### TREM2 Inhibiting Agent

One aspect of the present disclosure provides for targeting of TREM2, its receptor, or its downstream signaling. The present disclosure provides methods of enhancing checkpoint immunotherapy or reducing checkpoint immunotherapy resistance based on the discovery that blocking TREM2 results in tumor regression and immunostimulatory macrophage induction and/or immunosuppressive macrophage reduction.

As described herein, constructs of inhibitors of TREM2 (e.g., antibodies, fusion proteins, small molecules, peptides, functional fragments or variants thereof) can reduce or prevent checkpoint immunotherapy resistance. A TREM2 inhibiting agent can be any agent that can inhibit TREM2, downregulate TREM2, or knockdown TREM2. A TREM2 inhibiting agent can be a TREM2 antagonist. As an example, a TREM2 inhibiting agent can inhibit TREM2 signaling, activity, or function.

For example, the TREM2 inhibiting agent can be an anti-TREM2 antibody (e.g., anti-mTREM2). As an example, the anti-TREM2 antibody can be a native or Fc mutated anti-TREM2 antibody, or a functional fragment or variant thereof, such as a recombinant anti-TREM2 antibody or fusion protein. Furthermore, the anti-TREM2 antibody can be a chimeric antibody, a murine antibody, a humanized murine antibody, or a human antibody.

As another example, the TREM2 inhibiting agent can be a fusion protein. For example, the fusion protein can be a decoy receptor for TREM2.

As another example, a TREM2 inhibiting agent can be an inhibitory protein that antagonizes TREM2. For example, the TREM2 inhibiting agent can be a viral protein, which has been shown to antagonize TREM2.

As another example, a TREM2 inhibiting agent can be a short hairpin RNA (shRNA) or a short interfering RNA (siRNA) targeting TREM2.

As another example, a TREM2 inhibiting agent can be a sgRNA targeting TREM2.

Methods for preparing a TREM2 inhibiting agent (e.g., an agent capable of inhibiting TREM2 signaling) can comprise the construction of a protein/Ab scaffold containing the natural TREM2 receptor as a TREM2 neutralizing agent; developing inhibitors of the TREM2 receptor “down-stream”; or developing inhibitors of the TREM2 production “up-stream”.

Inhibiting TREM2 can be performed by genetically modifying TREM2 in a subject or genetically modifying a subject to reduce or prevent expression of the TREM2 gene, such as through the use of CRISPR-Cas9 or analogous technologies, wherein, such modification reduces or prevents TREM2 signaling, function, activity, or expression.

Inhibiting agents could also include small molecules that have the same effect, bind and inhibit or modulate TREM2, without depleting macrophages as a whole.

### Anti-TREM2 Constructs

As described herein, anti-TREM2 constructs (e.g., antibodies, proteins, etc.) specific for blocking TREM2, without killing myeloid cells (or not killing the immunosuppressive myeloid cells), and modulating macrophage effector function can be used in cancer therapy. The antibody construct can modulate macrophage functions, reducing their immunosuppressive functions and augmenting their immunostimulatory functions. The anti-TREM2 antibodies or proteins also include functional fragments, variants, mutants, recombinant antibodies, scFv, fusion proteins, Fc-fusion antibody-like proteins, peptides, etc., and humanized or chimeric variants thereof.

Previous work has shown the use of TREM2 antibodies with anti-PD1 therapies showing the reduction of tumor growth. But the currently known and previously disclosed TREM2 antibodies are directed at targeting and killing myeloid cells. Here, the antibodies target and block TREM2, do not kill myeloid cells, and result in loss of macrophage effector function (among other differences). Previous work uses targeting TREM2 as a cell-killing mechanism. Here, anti-TREM2 antibodies block function, but do not kill myeloid cells/macrophages, make tumor microenvironment hospitable to T-cells, recruit T-cells, have loss of effector function, and/or maintain immune system/recruit immune cells.

Here are described anti-TREM2 antibodies binding to immunoglobulin domain or stalk region, promoting recruitment of T-cells, not killing myeloid cells or macrophages, wherein the function includes modulation of TREM2 activity and loss of Fc effector function.

Uses can include: cancer therapy; enhanced immunostimulation; prevention of cytokine storm in checkpoint blockade therapy; prevention of cytokine release syndrome in CAR-T therapy; reducing checkpoint immunotherapy resistance; improving T-cell response; enhancing checkpoint immunotherapy efficacy; and to provide a safer therapy than killing/depleting all macrophages broadly. Anti-TREM2 antibodies can bind to the immunoglobulin domain and/or stalk region having functions/activities as described above.

Mutagenesis of anti-TREM2 antibodies and recombinant antibodies blocking TREM2 can be performed to enhance function or activity. CDR sequences for a recombinant antibody and variants/mutants thereof and a representative number of species, a % identity value, having a particular structure and function (e.g., block TREM2, do not kill macrophages, etc.) can be established by methods known in the art. Binding affinity values (Kd) and binding characteristics for immunoglobulin domain or stalk region binding or both to TREM2. Comparative experiments of prior work (e.g., TREM2 antibodies having myeloid killing function) can be established by methods known in the art.

In addition to checkpoint immunotherapies (such as anti-PD-1 therapies), other immunotherapies, such as CAR-T therapies, can also be combined with the anti-TREM2 blocking/inhibiting agents. Other anti-TREM2 antibodies without myeloid killing function (or other immunostimulatory function, etc.) can also be used as described herein.

Antibody fragments or variants having the desired activity or function can be as described in the art. The antibody molecule is modular and separate domains can be extracted through biochemical or genetic means. Novel, antigen-specific molecular forms are entering clinical evaluation. Therapeutics can be derived from antigen-specific fragments of antibodies produced by recombinant processes. Three general types of fragments can be, antigen-binding fragments (Fab), single chain variable fragments (scFv), and “third generation” (3G) (e.g., unibody), each representing a successive wave of antibody fragment technology. In parallel, drug developers can explore multi-specificity and conjugation with exogenous functional moieties in all three fragment types.

A TREM2 inhibiting agent can comprise an antibody or functional fragment or functional variant thereof having a percent identity to a TREM2 antibody and have or retain anti-TREM2 function or activity. A mrtation can also exist to reduce Fc effector function. For example, a TREM2 inhibiting agent can comprise about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100% identity to a functional fragment of an anti-TREM2 antibody.

### Types of Antibody Fragments

TREM2 antibodies functional fragments, or recombinant proteins thereof can be made and used clinically by methods known in the art (see e.g., Adam Bates and Christine A. Power, Review, David vs. Goliath: The Structure, Function, and Clinical Prospects of Antibody Fragments, Antibodies 2019, 8, 28) which can be designed to have the desired function or activity as discovered herein. TREM2 inhibiting agents, as described herein, can comprise an antibody fragment or variant (e.g., a fusion protein, scFv, peptide, recombinant proteins, diabodies, unibodies, etc., or a functional fragment, variant, or mutant (e.g., addition, insertion, deletion, substitution)) that have anti-TREM2 activity and reduced Fc effector function (e.g., Fc-mutated, loss of effector function mutation or variant).

F(ab′)2, Fab, Fab′ and Fv are antigen-binding fragments that can be generated from the variable region of IgG and IgM. These antigen-binding fragments can vary in size (MW), valency, or Fc content. Fc fragments can be generated entirely from the heavy chain constant region of an immunoglobulin. These and several additional unique fragment structures can be generated from pentameric IgM, including an “IgG”-type fragment, an inverted “IgG”-type fragment, and a pentameric Fc fragment.

Antibody effector functions are an important part of the humoral immune response and form an essential link between innate and adaptive immunity. Most of these effector functions are induced via the constant (Fc) region of the antibody, which can interact with complement proteins and specialized Fc-receptors.

Scheme 1. The names (nomenclature) and structures of some typical IgG fragments are illustrated in the following diagram and summarized below.

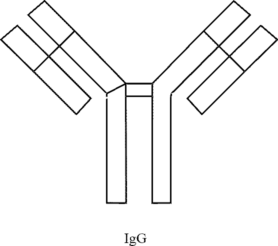

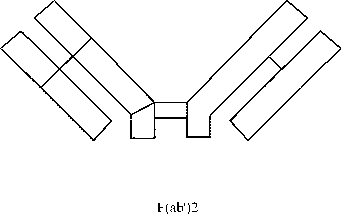

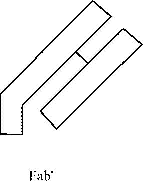

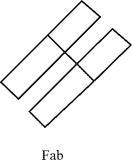

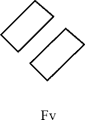

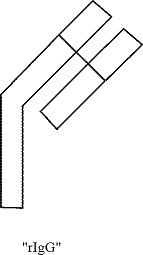

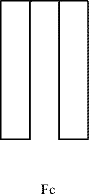

### F(ab′)2 Fragments

F(ab′)2 (110,000 daltons) fragments contain two antigen-binding regions joined at the hinge through disulfides. This fragment is void of most, but not all, of the Fc region.

### Fab′ Fragments

Fab′ (55,000 daltons) fragments can be formed by the reduction of F(ab′)2 fragments. The Fab′ fragment contains a free sulfhydryl group that may be alkylated or utilized in conjugation with an enzyme, toxin, or other protein of interest. Fab′ is derived from F(ab′)2; therefore, it may contain a small portion of Fc.

### Fab Fragments

Fab (50,000 daltons) is a monovalent fragment that is produced from IgG and IgM, consisting of the VH, CH1, and/or VL, CL regions, linked by an intramolecular disulfide bond.

### Fv Fragments

Fv (25,000 daltons) is the smallest fragment produced from IgG and IgM that contains a complete antigen-binding site. Fv fragments have the same binding properties and similar three-dimensional binding characteristics as Fab. The VH and VL chains of the Fv fragments are held together by non-covalent interactions. These chains tend to dissociate upon dilution, so methods have been developed to cross-link the chains through glutaraldehyde, intermolecular disulfides, or a peptide linker.

### “rlgG” Fragments

“rlgG” refers to reduced IgG (75,000 daltons) or half-IgG. It is the product of selectively reducing just the hinge-region disulfide bonds. Although several disulfide bonds occur in IgG, those in the hinge-region are the most accessible and easiest to reduce, especially with mild reducing agents like 2-mercaptoethylamine (2-MEA). Half-IgG can be prepared for the purpose of targeting the exposing hinge-region sulfhydryl groups that can be targeted for conjugation, either antibody immobilization or enzyme labeling.

### Fc Fragments

Fc (50,000 daltons) fragments contain the CH2 and CH3 region and part of the hinge region held together by one or more disulfides and noncovalent interactions. Fc and Fc5µ fragments are produced from fragmentation of IgG and IgM, respectively. The term Fc is derived from the ability of these antibody fragments to crystallize. Fc fragments are generated entirely from the heavy chain constant region of an immunoglobulin. The Fc fragment cannot bind antigen, but it is responsible for the effector functions of antibodies, such as complement fixation.

### Fc Mutations

As shown herein, mutations were made to the Fc region to reduce Fc effector function in immune cells. Loss of function mutations or mutations that reduce function can be done in a number of ways known in the art. For example, the region can be mutated, as shown herein, or mAbs can be produced in cells that add different glycans (see e.g., Kevin O. Saunders, Conceptual Approaches to Modulating Antibody Effector Functions and Circulation Half-Life, Front. Immunol., 07 Jun. 2019).

### Sequences

The signal sequence and the variable regions (Vh and VI) are highlighted in red. The rest of the sequences are constant regions (Ch1-H-Ch2-Ch3 and Cl).

21E10 (mouse anti-Human)
HC (SEQ ID NO: 1)
atgggttggagctgtatcatcttctttctggtagcaacagcta
caggtgtgcactccCAGGTCCAGCTGCAGCAGTCTGGGCCTGAGCTGGTG
AGGCCTGGGGTCTCAGTGAAGATTTCCTGCAAGGGTTCCGGCTACACATT
CACTGATTATGCTATGCACTGGGTGAAGCAGAGTCATGCAAAGAGTCTAG
AGTGGATTGGAGTTATTAGTACTTACTCTAGTAATACAAACTACAACCAG
AAGTTTAAGGGCAAGGCCACAATGACTGTAGACAAATCCTCCAGCACAGC
CTATATGGAACTTGCCAGATTGACATCTGAGGATTCTGCCATCTATTACT
GTGCAAGAGATGATGGTCACTACGTCTATGCTATGGACTACTGGGGTCAA
GGAACCTCAGTCACCGTCTCCTCAgccaaaacaacagccccatcggtcta
tccactggcccctgtgtgtggagatacaactggctcctcggtgactctag
gatgcctggtcaagggttatttccctgagccagtgaccttgacctggaac
tctggatccctgtccagtggtgtgcacaccttcccagctgtcctgcagtc
tgacctctacaccctcagcagctcagtgactgtaacctcgagcacctggc
ccagccagtccatcacctgcaatgtggcccacccggcaagcagcaccaag
gtggacaagaaaattgagcccagagggcccacaatcaagccctgrcctcc
atgcaaatgcccagcacctaacctcttgggtggaccatccgtcttcatct
tccctccaaagatcaaggatgtactcatgatctccctgagccccatagtc
acatgtgtggtggtggatgtgagcgaggatgacccagatgtccagatcag
ctggtttgtgaacaacgtggaagtacacacagctcagacacagacccata
gagaggattacaacagtactctccgggtggtcagtgccctccccatccag
caccaggactggatgagtggcaaggagttcaaatgcaaggtcaacaacaa
agacctcccagcgcccatcgagagaaccatctcaaaacccaaagggtcag
taagagctccacaggtatatgtcttgcctccaccagaagaagagatgact
aagaaacaggtcactctgacctgcatggtcacagacttcatgcctgaaga
catttacgtggagtggaccaacaacgggaaaacagagctaaactacaaga
acactgaaccagtcctggactctgatggttcttacttcatgtacagcaag
ctgagagtggaaaagaagaactgggtggaaagaaatagctactcctgttc
agtggtccacgagggtctgcacaatcaccacacgactaagagcttctccc
ggactccgggtaaatga

LC(k) (SEQ ID NO: 2)
atgaagctgcctgttctgctagtggtgctgctattgttcacgagtccagc
ctcaagcagtGATGTTGTTCTGACCCAAACTCCACTCTCTCTGCCTGTCA
ATATTGGAGATCAAGCCTCTATCTCTTGCAAGTCTACTAAGAGTCTTCTG
AATAGTGATGGATTCACTTATTTGGACTGGTACCTGCAGAAGCCAGGCCA
GTCTCCACAGCTCCTAATATATTTGGTTTCTAATCGATTTTCTGGAGTTC
CAGACAGGTTCAGTGGCAGTGGGTCAGGAACAGATTTCACACTCAAGATC
AGCAGAGTGGAGGCTGAGGATTTGGGAGTTTATTATTGCTTCCAGAGTAA
CTATCTTTACACGTTCGGAGGGGGACCAAGCTGGAAATAAAAcgggctga
tgctgcaccaactgtatccatcttcccaccatccagtgagcagttaacat
ctggaggtgcctcagtcgtgtgcttcttgaacaacttctaccccaaagac
atcaatgtcaagtggaagattgatggcagtgaacgacaaaatggcgtcct
gaacagttggactgatcaggacagcaaagacagcacctacagcatgagca
gcaccctcacgttgaccaaggacgagtatgaacgacataacagctatacc
tgtgaggccactcacaagacatcaacttcacccattgtcaagagcttcaa
caggaatgagtgttag

29E3 (mouse anti-Human)
HC (SEQ ID NO: 3)
atgggatggagctggatctttctcttcctcctgtcaggaactgcaggcgt
ccactctGAGGTCCAGTTTCAGCAGTCAGGACCTGAGCTGGTGAAACCTG
GGGCCTCAGTGAAGATATCCTGCAAGGCTTCTGGATACACATTCACTGAC
TACAACATGCACTGGGTGAAGCAGAGCCATGGAAAGAGCCTTGAGTGGAT
TGGATATATTTATCCTTACAATGGTGGGACTGGCTACAACCAGAAGTTCA
AGAGCAAGGCCACATTGACTGTAGACAATTCCTCCAGAACAGCCTACATG
GAACTCCGCAGCCTGTCATCTGAGGATTCTGCAGTCTATTACTGTGTAAG
AAGGGATAGGTACGACGACCCGTTTGTTTACTGGGGCCAAGGGACTCTGG
TCACTGTCTCTGCAgccaaaacaacagccccatcggtctatccactggcc
cctgtgtgtggagatacaactggctcctcggtgactctaggatgcctggt
caagggttatttccctgagccagtgaccttgacctggaactctggatccc
tgtccagtggtgtgcacaccttcccagctgtcctgcagtctgacctctac
accctcagcagctcagtgactgtaacctcgagcacctggcccagccagtc
catcacctgcaatgtggcccacccggcaagcagcaccaaggtggacaaga
aaattgagcccagagggcccacaatcaagccctgtcctccatgccaaatg
cccagcacctaacctcttgggtggaccatccgtcttcatcttccctccaa
agatcaaggatgtactcatgatctccctgagccccatagtcacatgtgtg
gtggtggatgtgagcgaggatgacccagatgtccagatcagctggtttgt
gaacaacgtggaagtacacacagctcagacacagacccatagagaggatt
acaacagtactctccgggtggtcagtgccctccccatccagcaccaggac
tggatgagtggcaaggagttcaaatgcaaggtcaacaacaaagacctccc
agcgcccatcgagagaaccatctcaaaacccaaagggtcagtaagagctc
cacaggtatatgtcttgcctccaccagaagaagagatgactaagaaacag
gtcactctgacctgcatggtcacagacttcatgcctgaagacatttacgt
ggagtggaccaacaacgggaaaacagagctaaactacaagaacactgaac
cagtcctggactctgatggttcttacttcatgtacagcaagctgagagtg
gaaaagaagaactgggtggaaagaaatagctactcctgttcagtggtcca
cgagggtctgcacaatcaccacacgactaagagcttctcccggactccgg
gtaaatga

LC(k) (SEQ ID NO: 4)
Atgaagcrgcctgttetgctsgtggtgcrgctsttgttcacgag
tccagcctcaagcagtGACATTGTGATGTCACAGTCTCCATCCTCCCTAG
CTGTGTCAGTTGGAGAGAAGGTTACTATGAGCTGCAAGTCCAGTCAGAGC
CTTTTATATAGTAGCAATCAAAAGAACTACTTGGCCTGGTACCAGCAGAA
ACCAGGGCAGTCTCCTAAACTGCTGATTTACTGGGCATCCACTAGGGAAT
CTGGGGTCCCTGATCGCTTCACAGGCAGTGGATCTGGGACAGATTTCACT
CTCACCATCAGCAGTGTGAAGGCTGCCTGGCAGTTTATTTCTGTCAGCAA
TATTATGGCTTTCCAATTCACGTTCGGCTCGGGGACAAACTTGGAAATAA
AGcgggctgatgctgcaccaactgtatccatcttcccaccatccagtgag
cagttaacatctggaggtgcctcagtcgtgtgcttcttgaacaacttcta
ccccaaagacatcaatgtcaagtggaagattgatggcagtgaacgacaaa
atggcgtcctgaacagttggactgatcaggacagcaaagacagcacctac
agcatgagcagcaccctcacgttgaccaaggacgagtatgaacgacataa
cagctatacctgtgaggccactcacaagacatcaacttcacccattgtca
agagcttcaacaggaatgagtgttag

178 (rat-anti-mouse)
HC (SEQ ID NO: 5)
atgaaatgcagctggatcaacctcttcttgatggcactagcttcaggggt
ctacgcaGAAGTACAGCTGCAGCAGTCTGGGCCCGAGCTTCGGAGACCTG
GGTCCTCAGTCAAGTTGTCTTGTAAGGCTTCTGGCTACAGTATTATAGAT
TACCTATGCACTGGGTAAAACATAGGCCAGGACACGGCCTGGAATGGATA
GGATGGATTGATCCTGAAAATGGTGAAACAAAATATGCTCAGAAGTTCCA
AAGGAAGGCCACACTGACTGCAGATACATCCTCCAGCACAGCCTACATGC
AACTCAGCAGCCTGACGTCTGAGGACACAGCAACCTATTTTTGTGCTAGA
CAACAACTACGGTATGTTATGGATGCCTGGGTCAAGGAGGTTCAGTCACT
GTCTCCTCAgccaaaacaacagccccatcggtctatccactggcccctgt
gtgtggagatacaactggctcctcggtgactctaggatgcctggtcaagg
gttatttccctgagccagtgaccttgacctggaactctggatccctgtcc
agtggtgtgcacaccttcccagctgtcctgcagtctgacctctacaccct
cagcagctcagtgactgtaacctcgagcacctggcccagccagtccatca
cctgcaatgtggcccacccggcaagcagcaccaaggtggacaagaaaatt
gagcccagagggcccacaatcaagccctgtcctccatgcaaatgcccagc
acctaacctcttgggtggaccatccgtcttcatcttccctccaaagatca
aggatgtactcatgatctccctgagccccatagtcacatgtgtggtggtg
gatgtgagcgaggatgacccagatgtccagatcagctggtttgtgaacaa
cgtggaagtacacacagctcagacacagacccatagagaggattacaaca
gtactctccgggtggtcagtgccctccccatccagcaccaggactggatg
agtggcaaggagttcaaatgcaaggtcaacaacaaagacctcccagcgcc
catcgagagaaccatctcaaaacccaaagggtcagtaagagctccacagg
tatatgtcttgcctccaccagaagaagagatgactaagaaacaggtcact
ctgacctgcatggtcacagacttcatgcctgaagacatttacgtggagtg
gaccaacaacgggaaaacagagctaaactacaagaacactgaaccagtcc
tggactctgatggttcttacttcatgtacagcaagctgagagtggaaaag
aagaactgggtggaaagaaatagctactcctgttcagtggtccacgaggg
tctgcacaatcaccacacgactaagagcttctcccggactccgggtaaat
ga

LC (SEQ ID NO: 6)
atgtcaggacacaatttagatatgagggtccagattcagttttgggggct
tcttctgctctggacatcaggtatacagtgtGATGTCCAGATGACCCAGT
CTCCATATAATCTTGCTGCCTCTCCTGGAGAAAGTGTTTCCATCAATTGC
AAGGCAAGTAAGAGCATTAGCAAGTATTTAGCCTGGTATCAACAGAAACC
TGGAAACAAATAAGCTTCTTATCTACGATGGGTCAACTTTGCAATCTGGA
ATTCCATCGAGGTTCAGTGGCAGTGGATCTGGTACAGATTTCACTCTCAC
CATCAGAAGCCTGGAGCCTGAAGATTTTGGGCTCTATTACTGTCAACAGC
ATAATGAATACCCGCTCACGTTCGGTTCTGGGACCAAGCTGGAGATCAAA
cgggctgatgctgcaccaactgtatctatcttcccaccatccacggaaca
gttagcaactggaggtgcctcagtcgtgtgcctcatgaacaacttctatc
ccagagacatcagtgtcaagtggaagattgatggcactgaacgacgagat
ggtgtcctggacagtgttactgatcaggacagcaaagacagcacgtacag
catgagcagcaccctctcgttgtccaaggctgactatgaaagtcataacc
tctatacctgtgaggttgttcataagacatcatcctcacccgtcgtcaag
agcttcaacaggaatgagtgttag

### Immune Cells

As shown herein, the TREM2 antibodies do not deplete total macrophage cells. It has been shown herein that the disclosed antibody constructs remodel the macrophage/myelod population (e.g., tumor infiltrating cells). Immunosuppressive and immunostimulating myeloids can be characterized functionally. For example, immunosuppressive cells block T cell responses. As another example, correlative markers of immunosuppressive activity can include MRC1 (CD206). As another example, immunostimulating cells activate T cell responses. As another example, correlative markers of immunostimulation include iNOS (NOS2).

### Fc Receptors

Fc receptors (FcRs) are key immune regulatory receptors connecting the antibody mediated (humoral) immune response to cellular effector functions. Receptors for all classes of immunoglobulins have been identified, such as FcyR (IgG), FcεRI (IgE), FcαRI (IgA), FcµR (IgM) and FcδR (IgD). There are three classes of receptors for human IgG found on leukocytes: CD64 (FcyRI), CD32 (FcyRlla, FcyRllb, and FcyRllc) and CD16 (FcyRllla and FcyRlllb). FcyRI is classed as a high affinity receptor (nanomolar range KD) while FcyRll and FcyRlll are low to intermediate affinity (micromolar range KD).

In antibody dependent cellular cytotoxicity (ADCC), FcvRs on the surface of effector cells (e.g., natural killer cells, macrophages, monocytes, eosinophils) bind to the Fc region of an IgG which itself is bound to a target cell. Upon binding a signalling pathway is triggered which results in the secretion of various substances, such as lytic enzymes, perforin, granzymes, or tumor necrosis factor, which can mediate in the destruction of the target cell. The level of ADCC effector function varies for IgG subtypes. Although this is dependent on the allotype and specific FcvR, generally, ADCC effector function is high for human IgG1 and IgG3, and low for IgG2 and IgG4. FcγRs can bind to IgG asymmetrically across the hinge and upper CH2 region. Knowledge of the binding site has resulted in engineering efforts to modulate IgG effector functions.

Here, antibodies without Fc effector function were studied. It has been shown here that modifying this effector function, in combination with anti-TREM2 activity, can result in macrophage population remodeling in order to enhance efficacy of immunotherapies.

### Immunotherapy

As described herein, TREM2 can be targeted in combination with a number of therapies, such as immunotherapy (e.g., CAR-T) or checkpoint immunotherapy.

### Checkpoint Immunotherapy

An important function of the immune system is its ability to tell between normal cells in the body and those it sees as “foreign.” This lets the immune system attack the foreign cells while leaving the normal cells alone. To do this, it uses “checkpoints.” Immune checkpoints are molecules on certain immune cells that need to be activated (or inactivated) to start an immune response.

Cancer cells can find ways to use these checkpoints to avoid being attacked by the immune system. But drugs that target these checkpoints hold a lot of promise as a cancer treatment. These drugs are called checkpoint inhibitors. Checkpoint inhibitors used to treat cancer don’t work directly on the tumor at all. They only take the brakes off an immune response that has begun but hasn’t yet been working at its full force.

Checkpoint immunotherapy has been extensively shown to unleash T cell effector functions to control tumors in both ice and many cancer patients. However, tumor cells can evade immunological elimination by recruiting myeloid cells that induce an immunosuppressive state. Recent high dimensional profiling studies have shown that tumor-infiltrating myeloid cells are considerably heterogeneous, and may include both immunostimulatory and immunosuppressive subsets, although they do not fit the M1/M2 paradigm. Thus, depletion of suppressive myeloid cells from tumors, blockade of their functions, or induction of myeloid cells with immunostimulatory properties may provide important approaches for improving immunotherapy strategies, perhaps in synergy with checkpoint blockade.

Any immune checkpoint inhibitor known in the art can be used. For example, a PD-1 inhibitor can be used. These drugs are typically administered IV (intravenously). PD-1 is a checkpoint protein on immune cells called T cells. It normally acts as a type of “off switch” that helps keep the T cells from attacking other cells in the body. It does this when it attaches to PD-L1, a protein on some normal (and cancer) cells. When PD-1 binds to PD-L1, it tells the T cell to leave the other cell alone. Some cancer cells have large amounts of PD-L1, which helps them hide from an immune attack.

Monoclonal antibodies that target either PD-1 or PD-L1 can block this binding and boost the immune response against cancer cells. These drugs have shown a great deal of promise in treating certain cancers.

Examples of drugs that target PD-1 can include: Pembrolizumab (Keytruda), Nivolumab (Opdivo), or Cemiplimab (Libtayo). These drugs have been shown to be helpful in treating several types of cancer, and new cancer types are being added as more studies show these drugs to be effective.

As another example, a PD-L1 inhibitor can be used. Examples of drugs that target PD-L1 can include: Atezolizumab (Tecentriq), Avelumab (Bavencio), or Durvalumab (Imfinzi). These drugs have also been shown to be helpful in treating different types of cancer, and are being studied for use against others.

CTLA-4 is another protein on some T cells that acts as a type of “off switch” to keep the immune system in check. For example, Ipilimumab (Yervoy) is a monoclonal antibody that attaches to CTLA-4 and reduces or blocks its function. This can boost the body’s immune response against cancer cells. This drug can be used to treat melanoma of the skin and other cancers.TREM2

TREM2 is an activating receptor of the Ig-superfamily that binds lipids and transmits intracellular protein tyrosine phosphorylation signals. We and others have previously demonstrated that TREM2 sustain microglial responses to Alzheimer’s Disease. Additional studies have highlighted TREM2 expression also in peripheral tissue macrophages, such as liver and adipose tissue, where macrophages contribute to fibrosis and metabolism, as well as tumor macrophages. However, the impact of TREM2 on tumors is unknown. In the present paper, we demonstrate that TREM2 is not only a major marker of tumor infiltrating macrophages in mouse models and human tumors, but is also protumorigenic, skewing the landscape of tumor infiltrating macrophages towards immunosuppression.

By modifying tumor macrophage landscape, TREM2 blockade attenuates tumor growth and facilitates checkpoint immunotherapy. Altogether, our study provides the first demonstration that TREM2 blockade is an attractive approach for effectively augmenting checkpoint immunotherapy by modifying the immunosuppressive myeloid cell infiltrate of tumors.

### Cancer

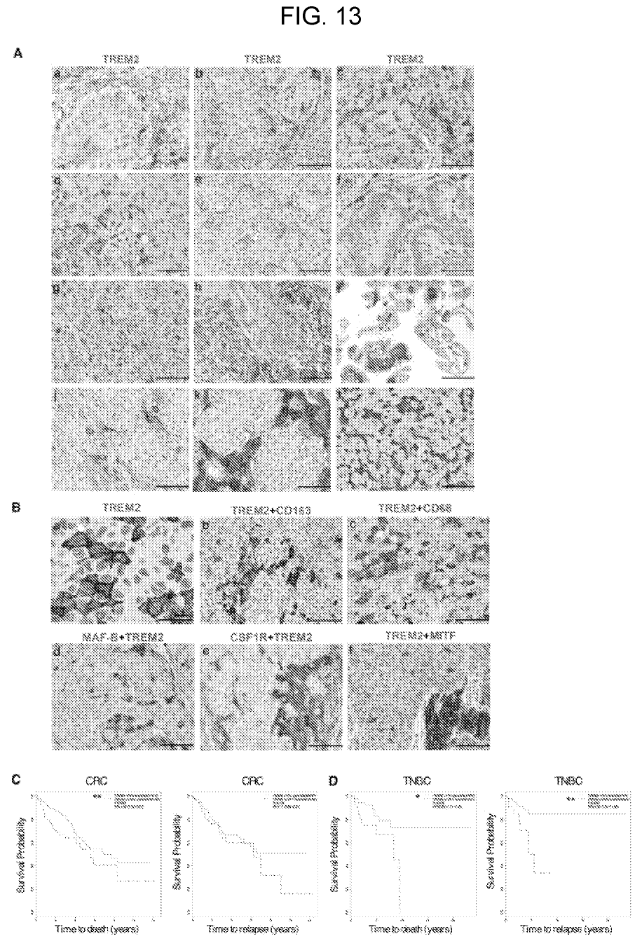

TREM2 is a marker of infiltrating macrophages in over 200 human tumors. Numerous tumors are infiltrated by TREM2+ infiltrating cells (see e.g., FIG. 13). The TCGA database was further interrogated to investigate negative correlation between TREM2 expression and survival for sarcomas, breast cancers, colorectal carcinomas, and lung adenocarcinomas. It was shown CRC and TNBC showed a negative correlation, although all the tumors in the TCGA database (several hundreds) were not screened.

TREM2 expression was shown here to correlate with tumor-associated macrophage (TAM) genes in cancer patients. In many tumor types TAM infiltration level has been shown to be of significant prognostic value. TREM2 inhibiting agents have been shown to modulate the macrophage microenvironment to increase immunostimulatory macrophages and decrease the immunosuppressive macrophages in tumors. As an example, here a sarcoma, colorectal, and breast mouse models were studies and in humans, a negative correlation was shown between levels of TREM2 and prognosis in colorectal carcinoma and triple negative breast cancer.

Methods and compositions as described herein can be used for the prevention, treatment, or slowing the progression of cancer or tumor growth. The cancer can be associated with tumors having TREM2+ infiltrating macrophages (TREM2-associated cancer or tumor). For example, the cancer can be Acute Lymphoblastic Leukemia (ALL); Acute Myeloid Leukemia (AML); Adrenocortical Carcinoma; AIDS-Related Cancers; Kaposi Sarcoma (Soft Tissue Sarcoma); AIDS-Related Lymphoma (Lymphoma); Primary CNS Lymphoma (Lymphoma); Anal Cancer; Appendix Cancer; Gastrointestinal Carcinoid Tumors; Astrocytomas; Atypical Teratoid/Rhabdoid Tumor, Childhood, Central Nervous System (Brain Cancer); Basal Cell Carcinoma of the Skin; Bile Duct Cancer; Bladder Cancer; Bone Cancer (including Ewing Sarcoma and Osteosarcoma and Malignant Fibrous Histiocytoma); Brain Tumors; Breast Cancer; Bronchial Tumors; Burkitt Lymphoma; Carcinoid Tumor (Gastrointestinal); Childhood Carcinoid Tumors; Cardiac (Heart) Tumors; Central Nervous System cancer; Atypical Teratoid/Rhabdoid Tumor, Childhood (Brain Cancer); Embryonal Tumors, Childhood (Brain Cancer); Germ Cell Tumor, Childhood (Brain Cancer); Primary CNS Lymphoma; Cervical Cancer; Cholangiocarcinoma; Bile Duct Cancer Chordoma; Chronic Lymphocytic Leukemia (CLL); Chronic Myelogenous Leukemia (CML); Chronic Myeloproliferative Neoplasms; Colorectal Cancer; Craniopharyngioma (Brain Cancer); Cutaneous T-Cell; Ductal Carcinoma In Situ (DCIS); Embryonal Tumors, Central Nervous System, Childhood (Brain Cancer); Endometrial Cancer (Uterine Cancer); Ependymoma, Childhood (Brain Cancer); Esophageal Cancer; Esthesioneuroblastoma; Ewing Sarcoma (Bone Cancer); Extracranial Germ Cell Tumor; Extragonadal Germ Cell Tumor; Eye Cancer; Intraocular Melanoma; Intraocular Melanoma; Retinoblastoma; Fallopian Tube Cancer; Fibrous Histiocytoma of Bone, Malignant, or Osteosarcoma; Gallbladder Cancer; Gastric (Stomach) Cancer; Gastrointestinal Carcinoid Tumor; Gastrointestinal Stromal Tumors (GIST) (Soft Tissue Sarcoma); Germ Cell Tumors; Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease; Hairy Cell Leukemia; Head and Neck Cancer; Heart Tumors; Hepatocellular (Liver) Cancer; Histiocytosis, Langerhans Cell; Hodgkin Lymphoma; Hypopharyngeal Cancer; Intraocular Melanoma; Islet Cell Tumors; Pancreatic Neuroendocrine Tumors; Kaposi Sarcoma (Soft Tissue Sarcoma); Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Lymphoma; Male Breast Cancer; Malignant Fibrous Histiocytoma of Bone or Osteosarcoma; Melanoma; Melanoma, Intraocular (Eye); Merkel Cell Carcinoma (Skin Cancer); Mesothelioma, Malignant; Metastatic Cancer; Metastatic Squamous Neck Cancer with Occult Primary; Midline Tract Carcinoma Involving NUT Gene; Mouth Cancer; Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip or Oral Cavity Cancer; Oropharyngeal Cancer; Osteosarcoma and Malignant Fibrous Histiocytoma of Bone; Ovarian Cancer Pancreatic Cancer; Pancreatic Neuroendocrine Tumors (Islet Cell Tumors); Papillomatosis; Paraganglioma; Paranasal Sinus and Nasal Cavity Cancer; Parathyroid Cancer; Penile Cancer; Pharyngeal Cancer; Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer Renal Cell (Kidney) Cancer; Retinoblastoma; Rhabdomyosarcoma, Childhood (Soft Tissue Sarcoma); Salivary Gland Cancer; Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Uterine Sarcoma; Sezary Syndrome (Lymphoma); Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; T-Cell Lymphoma, Cutaneous; Lymphoma; Mycosis Fungoides and Sèzary Syndrome; Testicular Cancer; Throat Cancer; Nasopharyngeal Cancer; Oropharyngeal Cancer; Hypopharyngeal Cancer; Thymoma and Thymic Carcinoma; Thyroid Cancer; Thyroid Tumors; Transitional Cell Cancer of the Renal Pelvis and Ureter (Kidney (Renal Cell) Cancer); Ureter and Renal Pelvis; Transitional Cell Cancer (Kidney (Renal Cell) Cancer; Urethral Cancer; Uterine Cancer, Endometrial; Uterine Sarcoma; Vaginal Cancer; Vascular Tumors (Soft Tissue Sarcoma); Vulvar Cancer; or Wilms Tumor.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent (%) sequence identity = X/Y100, where X is the number of residues scored as identical matches by the sequence alignment program’s or algorithm’s alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6 X SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6 X SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ = 81.5° C. + 16.6(log₁₀[Na⁺]) + 0.41 (fraction G/C content) - 0.63(% formamide) - (600/I). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| | |
|---|---|
| Side Chain Characteristic | Amino Acid |
| Aliphatic Non-polar | GAPILV |
| Polar-uncharged | CSTMNQ |
| Polar-charged | DEKR |
| Aromatic | HFWY |
| Other | NQDE |

Conservative Substitutions II

| Side Chain Characteristic | Amino Acid |
|---|---|
| Non-polar (hydrophobic) | |
| A. Aliphatic: | ALIVP |
| B. Aromatic: | FW |
| C. Sulfur-containing: | M |
| D. Borderline: | G |
| A. Hydroxyl: | STY |
| B. Amides: | N Q |
| C. Sulfhydryl: | C |
| D. Borderline: | G |
| Positively Charged (Basic): | KRH |
| Negatively Charged (Acidic): | DE |

Conservative Substitutions III

| | |
|---|---|
| Original Residue | Exemplary Substitution |
| Ala (A) | Val, Leu, lIe |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| lIe (I) | Leu, Val, Met, Ala, Phe, |
| Leu (L) | lIe, Val, Met, Ala, Phe |
| Lys (K) | Arg, Gln, Asn |
| Met(M) | Leu, Phe, lIe |
| Phe (F) | Leu, Val, lIe, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | lIe, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326 -330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Formulation

The agents and compositions described herein can be formulated by any conventional manner using one or more pharmaceutically acceptable carriers or excipients as described in, for example, Remington’s Pharmaceutical Sciences (A.R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington’s Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating cancer in a subject in need of administration of a therapeutically effective amount of a TREM2 inhibiting agent, so as to reduce checkpoint immunotherapy resistance, enhance checkpoint immunotherapy efficacy, induce immunostimulatory macrophages, and/or reduce immunosuppressive macrophages.

Methods described herein are generally performed on a subject in need thereof. A subject in need of the therapeutic methods described herein can be a subject having, diagnosed with, suspected of having, or at risk for developing cancer or a tumor. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a TREM2 inhibiting agent is, for example, an amount that would cause the desired therapeutic effect in a subject while minimizing undesired side effects. In various embodiments, an effective amount of a TREM2 inhibiting agent described herein can substantially inhibit cancer or tumor growth, slow the progress of cancer or tumor growth, or limit the development of tumor growth.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a TREM2 inhibiting agent can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce checkpoint immunotherapy resistance, enhance checkpoint immunotherapy efficacy, induce immunostimulatory macrophages, and/or reduce immunosuppressive macrophages.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of a TREM2 inhibiting agent can occur as a single event or over a time course of treatment. For example, a TREM2 inhibiting agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for the treatment of cancer.

A TREM2 inhibiting agent can be administered simultaneously or sequentially with another agent, such as a checkpoint inhibitor, an immunotherapy, an anti-cancer agent, an antibiotic, an anti-inflammatory, or another agent. For example, a TREM2 inhibiting agent can be administered simultaneously with another agent, such as a checkpoint inhibitor, an immunotherapy, an anti-cancer agent, an antibiotic, or an anti-inflammatory. Simultaneous administration can occur through administration of separate compositions, each containing one or more of a TREM2 inhibiting agent, a checkpoint inhibitor, an immunotherapy, an anti-cancer agent, an antibiotic, an anti-inflammatory, or another agent. Simultaneous administration can occur through administration of one composition containing two or more of a TREM2 inhibiting agent, a checkpoint inhibitor, an immunotherapy, an anti-cancer agent, an antibiotic, an anti-inflammatory, or another agent. A TREM2 inhibiting agent can be administered sequentially with an antibiotic, an anti-inflammatory, or another agent. For example, a TREM2 inhibiting agent can be administered before or after administration of a checkpoint inhibitor, an immunotherapy, an anti-cancer agent, an antibiotic, an anti-inflammatory, or another agent.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 µm), nanospheres (e.g., less than 1 µm), microspheres (e.g., 1-100 µm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: TREM2 Modulation Remodels the Tumor Myeloid Landscape Enhancing Anti-PD-1 Immunotherapy

This example describes methods and compositions for reducing resistance to, or enhancing, checkpoint inhibitor therapy.

TREM2 is a pro-tumorigenic marker of tumor-infiltrating macrophages in mouse models and human tumors that can be targeted to curb tumor growth and improve the efficacy of checkpoint blockade therapy while remodeling the landscape of tumor-infiltrating macrophages.

As shown herein, TREM2 is expressed by tumor-associated macrophages in different types of tumors; TREM2 deficiency and anti-TREM2 mAb treatment both curb tumor growth in mice; anti-PD-1 treatment is more efficacious when TREM2 is either absent or engaged by a mAb; and modulation of TREM2 remodels the tumor macrophage landscape.

### Summary

Checkpoint immunotherapy unleashes T cell control of tumors, but is undermined by immunosuppressive myeloid cells. TREM2 is a myeloid receptor that transmits intracellular signals that sustain microglial responses during Alzheimer’s disease. TREM2 is also expressed by tumor-infiltrating macrophages. Here, we found that Trem2^{-/-} mice are more resistant to growth of various cancers than wild-type mice and are more responsive to anti-PD-1 immunotherapy. Furthermore, treatment with anti-TREM2 mAb curbed tumor growth and fostered regression when combined with anti-PD-1. scRNA-seq revealed that both TREM2 deletion and anti-TREM2 are associated with scant MRC1⁺ and CX₃CR1⁺ macrophages in the tumor infiltrate, paralleled by expansion of myeloid subsets expressing immunostimulatory molecules that promote improved T cell responses. TREM2 was expressed in tumor macrophages in over 200 human cancer cases and inversely correlated with prolonged survival for two types of cancer. Thus, it was determined that TREM2 could n be targeted to modify tumor myeloid infiltrates and augment checkpoint immunotherapy.

### Introduction

The immune system plays an important protective function against tumor development and progression, effectively eliminating immunogenic cancer cells (Schreiber et al., 2011). To escape immunosurveillance, cancer cells muffle their immunogenic features and induce a microenvironment that actively suppresses immune responses. Suppressive mechanisms directly affect T cell responses by engaging immune checkpoints such as cytotoxic T-lymphocyte associated antigen-4 (CTLA-4) and programmed death-1 (PD-1) (Freeman et al., 2000; Leach et al., 1996). Tumors also coopt myeloid cells to actively suppress anti-tumor T cell responses. Myeloid cells constitute a significant cellular fraction of the microenvironment of many tumors and have been shown to inhibit T cell responses through multiple mechanisms (Mantovani et al., 2017). Although collectively considered suppressor cells, recent high-dimensional profiling studies have shown that tumor-infiltrating myeloid cells are considerably heterogeneous and may in fact include both immunostimulatory and immunosuppressive subsets (Broz and Krummel, 2015; Cassetta and Pollard, 2018; Gubin et al., 2018; Lavin et al., 2017). Immunostimulatory myeloid cells include type 1 dendritic cells (DC1s) and M1-like IFN-γ-induced macrophages; suppressive myeloid cells include M2-like macrophages, as well as a heterogeneous group of myeloid progenitor cells and immature myeloid cells collectively defined as myeloid-derived suppressor cells (MDSCs) (Veglia et al., 2018). Thus, depletion of suppressive myeloid cells from tumors, blockade of their functions, or induction of myeloid cells with immunostimulatory properties may constitute important approaches for improving immunotherapy strategies, perhaps in synergy with checkpoint blockade (Elinav et al., 2013).

Recently, attention has been focused on unique subsets of macrophages expressing the cell surface receptor TREM2. TREM2 is an activating receptor of the Ig-superfamily that binds lipids and transmits intracellular signals through the adaptor DAP12 (Peng et al., 2010; Ulland et al., 2017). DAP12 recruits the protein tyrosine kinase Syk, which initiates a cascade of tyrosine phosphorylation events that activate downstream mediators such as PLCy2, PI-3K, Vav, mTOR, and MAPK, ultimately leading to cell activation (Peng et al., 2010; Ulland et al., 2017). Although TREM2 is expressed on the cell surface, it is cleaved from the cell surface by metalloproteases and released as soluble TREM2 (sTREM2) (Ulland and Colonna, 2018). TREM2 cleavage may regulate cell activation; moreover, soluble TREM2 has been proposed to promote survival of neighboring cells (Zhong et al., 2017).

TREM2 has been extensively studied in microglia for its capacity to sustain microglial responses to neurodegenerative pathologies, such as Alzheimer’s disease (Ulland and Colonna, 2018). However, TREM2 is also expressed in several peripheral macrophage populations involved in host defense and metabolism. During lung acute viral infection, TREM2⁺ macrophages release sTREM2, which inhibits apoptosis of macrophages, causing a feed-forward expansion of lung macrophages that converts acute infection into a chronic inflammatory disease (Wu et al., 2015). In the adipose tissue, TREM2 sustains the presence of a population of lipid-associated macrophages (LAMs) that prevent the dysmetabolism engendered by a high-fat diet (Jaitin et al., 2019). In atherosclerosis, TREM2⁺ macrophages are enriched in atherosclerotic lesions and specialize in lipid catabolism (Cochain et al., 2018). In the liver, a TREM2⁺CD9⁺ subset of macrophages that differentiate from circulating monocytes expands during liver cirrhosis and contributes to fibrosis (Ramachandran et al., 2019). In the skin, TREM2⁺ dermal macrophages secrete oncostatin M, which inhibits hair growth by maintaining hair follicle stem cells in a quiescent state (Wang et al., 2019). TREM2⁺ macrophages have also been reported in tumors (Lavin et al., 2017; Song et al., 2019), but the impact of TREM2 in tumor immune responses has not been addressed.

Here, we found that Trem2^{-/-} mice are more resistant to tumor growth than wild-type (WT) mice using 3-methylcholanthrene (MCA)-induced sarcoma, colorectal cancer, and mammary tumor models. TREM2 deficiency was associated with alterations in macrophage subsets and an increase of intratumoral CD8⁺ T cells, some of which expressed PD-1. This observation prompted us to ask whether TREM2 blockade can enhance antitumor responses mediated by checkpoint immunotherapy. First, we showed that anti-PD-1 immunotherapy is more effective in TREM2-deficient than WT tumor-bearing mice. Moreover, administration of an Fc-mutated anti-TREM2 monoclonal antibody (mAb) to tumor-bearing mice blunted tumor growth and strongly enhanced the efficacy of anti-PD-1 immunotherapy. Analysis of the myeloid cell landscape by single-cell RNA sequencing (scRNA-seq) showed that both TREM2 deficiency and anti-TREM2 mAb treatment triggered marked changes in the macrophage populations infiltrating the tumor: CX₃CR1⁺ and MRC1⁺ macrophage subsets declined, while novel subsets expressing potentially immunostimulatory molecules were induced. In parallel with the mouse data, we found that TREM2 is a marker of infiltrating macrophages in over 200 human tumors examined by immunohistochemistry (IHC). Moreover, TREM2 expression inversely correlated with greater overall survival and relapse-free survival in colorectal carcinoma (CRC) and triple-negative breast cancer (TNBC). We conclude that reshaping of tumor-associated macrophages by anti-TREM2 mAb is a promising avenue for complementing checkpoint immunotherapy.

### Results

### TREM2 Deficiency Delays Growth of Transplanted Tumors and Modifies the Tumor-Immune Infiltrate

To address the potential impact of TREM2 on immune responses to tumors, we chose mouse tumor models known to be associated with a microenvironment infiltrated by macrophages expressing TREM2. We first analyzed an MCA-induced sarcoma cell line (MCA/1956) in WT and Trem2^{-/-} mice. MCA/1956 belongs to a panel of MCA-induced sarcomas known as progressors. Since these tumors were developed in immunocompetent WT mice, their immunogenic profiles were edited by the immune system, and hence they grow unopposed when transplanted into naive syngeneic WT hosts (Alspach et al., 2019; Schreiber et al., 2011; Shankaran et al., 2001). These tumors have recently been shown to be infiltrated by a variety of macrophage subsets (Gubin et al., 2018), many of which express TREM2 (unpublished data).

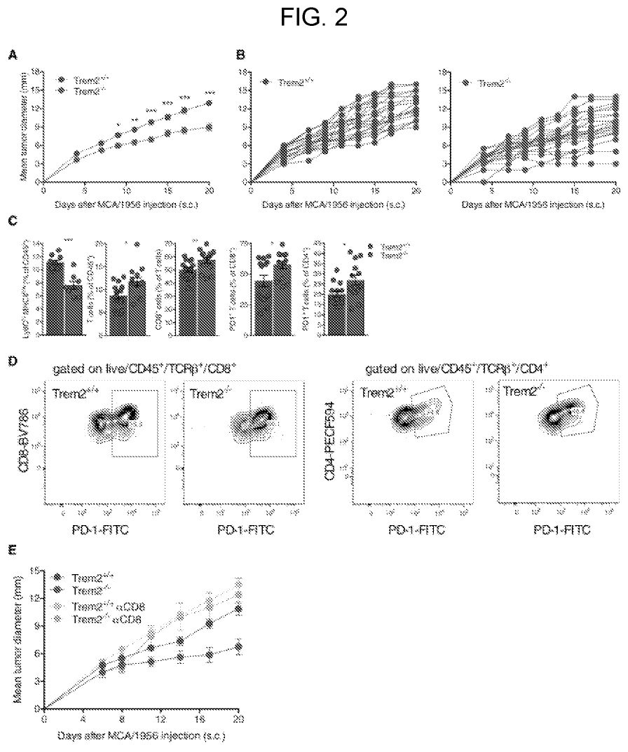

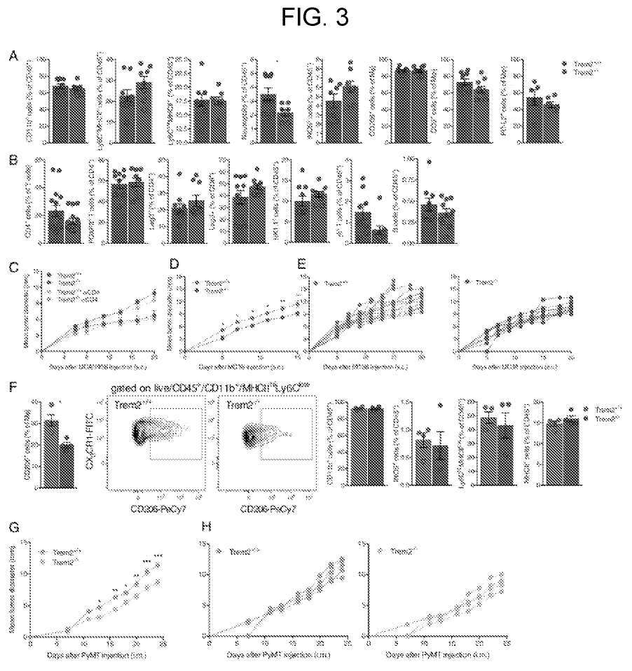

MCA/1956 grew progressively in WT mice, but was consistently attenuated in Trem2^{-/-} mice (FIG. 2A and FIG. 2B). We then examined the immune infiltrate of MCA/1956 tumors in WT and Trem2^{-/-} mice 10 days after tumor injection by flow cytometry. Total CD11b^{hi} myeloid cells were equally represented in WT and Trem2^{-/-} infiltrates (FIG. 3A); however, the relative proportion of a Ly6C⁺MHC∥^{low/-} subset was significantly reduced in the Trem2^{-/-} infiltrates (FIG. 2C), while the Ly6C⁺MHC∥⁺ and Ly6C⁻MHC∥⁺ subsets were present at similar frequencies (FIG. 3A), suggesting that TREM2 deficiency may impact the myeloid infiltrate. Trem2^{-/-} tumor infiltrates also contained more TCRβ⁺ T cells and CD8⁺ T cells than did WT infiltrates, in line with more robust tumor control in Trem2^{-/-} mice (FIG. 2C). In addition, significantly more CD8⁺ T cells and CD4⁺ T cells expressed PD-1 in the Trem2^{-/-} infiltrates (FIG. 2C and FIG. 2D), suggesting that TREM2 deficiency may promote increased T cell activation and potentially enhance responsiveness to anti-PD-1 checkpoint blockade. Lag3⁺ T cells, Tregs, and other lymphoid cells such as natural killer (NK) cells, yδ T cells, and B cells were equally represented in tumor infiltrates from WT and Trem2^{-/-} mice (FIG. 3B). To directly address the impact of T cells on the restrained tumor growth observed in Trem2^{-/-} mice, we depleted CD8⁺ T cells and CD4⁺ T cells in tumor-bearing Trem2^{+/+} and Trem2^{-/-} mice. Upon depletion of CD8⁺ T cells (FIG. 2E), but not CD4⁺ T cells (FIG. 3C), tumor growth was no longer attenuated, but in fact accelerated in Trem2^{-/-} mice. Collectively, these data suggested that TREM2 deficiency modifies the MCA myeloid microenvironment in a manner that facilitates partial control of tumor growth by CD8⁺ T cells. Moreover, it appeared that CD8⁺ T cells generated in the TREM2-deficient environment may be more responsive to anti-PD-1.

We extended our analysis to the growth of MC38 colorectal cancer, which has been shown to depend on the function of infiltrating macrophages (Rashidian et al., 2019). Meta-analyses of published RNA-seq data from the MC38 model (Arlauckas et al., 2018; Hoves et al., 2018) showed that macrophages express TREM2 (data not shown). Consistent with our results with the MCA/1956 model, MC38 tumor growth was more subdued in Trem2^{-/-} mice than in WT mice (FIG. 3D and FIG. 3E). Flow cytometric analysis of the tumor infiltrate of MC38 showed that TREM2 deficiency was associated with reduced representation of yet another subset of macrophages expressing CD206 (FIG. 3F) that have been previously involved in immunosuppression (Allavena et al., 2010; Mantovani et al., 2017), whereas no obvious phenotypic changes were observed in the T cell compartment (data not shown). Finally, we assessed the expansion of PyMT mammary tumors, which are infiltrated by macrophages that have been shown to promote growth and metastatic potential (Franklin et al., 2014; Lin et al., 2001) and to express TREM2 (Ojalvo et al., 2009). Once again, tumor growth was more limited in Trem2^{-/-} mice than in WT mice (FIG. 3G and FIG. 3H). Cumulatively, these results demonstrated that TREM2 deficiency consistently restricts the growth of various tumors that are infiltrated by macrophages; moreover, flow cytometric analyses hinted that this restraint is associated with changes in infiltrating macrophages that may impact T cell responses.

### Lack of TREM2 Impacts Tumor Myeloid and Lymphoid Landscape

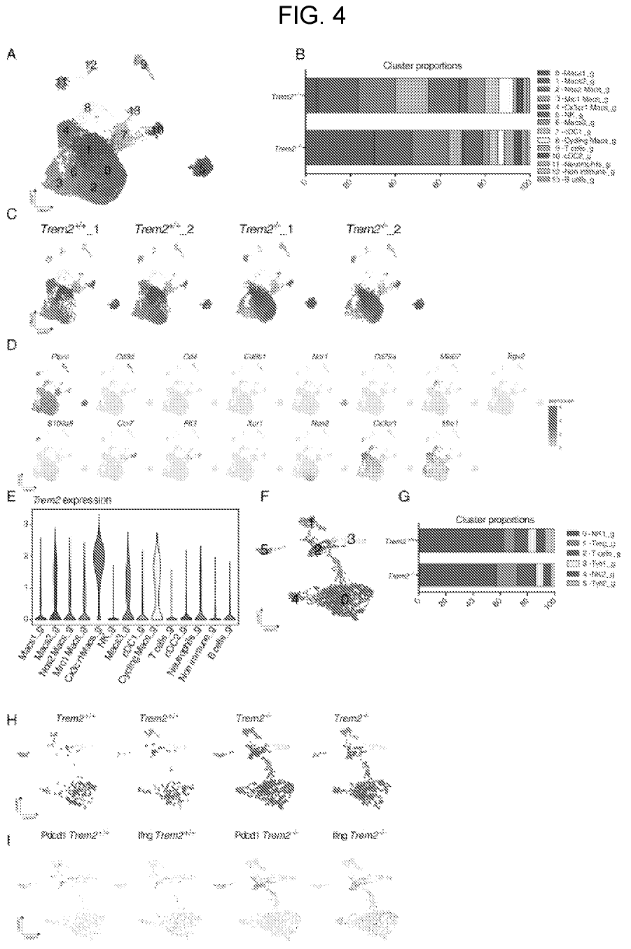

To define the impact of TREM2 deficiency on the tumor-immune infiltrate at higher resolution, we analyzed immune cells in WT and Trem2^{-/-} MCA/1956 tumors by scRNA-seq. We sorted live-CD45⁺ cells from MCA/1956 tumors 10 days after tumor injection (FIG. 4A and FIG. 4B). We chose this time point to detect early changes in the myeloid compartment related to lack of TREM2, before these changes become entwined with altered T cell responses and modified pathology. We obtained data for an average of 4,940 cells per sample with a coverage of 13,545 UMIs (unique molecular identifiers) per cell. Unsupervised clustering by Uniform Manifold Approximation and Projection (UMAP) identified 14 clusters (FIG. 4A-FIG. 4D). Expression of Ptprc (CD45) was evident in all clusters, with the exception of a few cells (cluster 12), which included contaminating non-immune cells. Cluster 9 expressed T cell markers (Cd3d, Cd4, and Cd8b1), while cluster 5 encompassed NK cells (Ncr1). Cluster 13 (Cd79a) and cluster 11 (S100a8) identified B cells and neutrophils, respectively. Cluster 8 corresponded to Mki67⁺ proliferating cells, predominantly CX₃CR1⁺ macrophages. Cluster 10 included DC2-like cells based on the expression of Flt3 and Ccr7, while cluster 7 represented Xcr1⁺ DC1-like cells (FIG. 4D).

Macrophage subsets were grouped in clusters 0, 1, 2, 3, 4, 6, and 8 (FIG. 4A-FIG. 4D). Some clusters were designated based on the expression of a characteristic marker such as CX₃CR1-Macs-g, Mrc1-Macs-g, Nos2-Macs-g, and Cycling-Macs-g (Mki67⁺). Other clusters were simply identified as Macs-1-3-g, as they expressed a constellation of markers rather than an archetypal macrophage marker. All clusters were further labeled with a “g” to indicate that they were identified in a comparison between tumor-bearing mice with different genotypes (WT and Trem2^{-/-}). We noticed that CX₃CR1-Macs-g, Mrc1-Macs-g, Cycling-Macs-g, and Macs3-g were relatively meagerly represented in Trem2^{-/-} tumors, whereas the Macs1-g cluster predominated (FIG. 4B-FIG. 4D). Moreover, T cells (cluster 9) and NK cells (cluster 5) were enriched in Trem2^{-/-} mice (FIG. 4B and FIG. 4C). This initial evaluation established that lack of TREM2 impacts both myeloid and lymphoid tumor infiltrates. Moreover, TREM2 was expressed in all macrophage clusters, although at different levels, while it was absent in DCs and lymphoid cells (FIG. 4E).

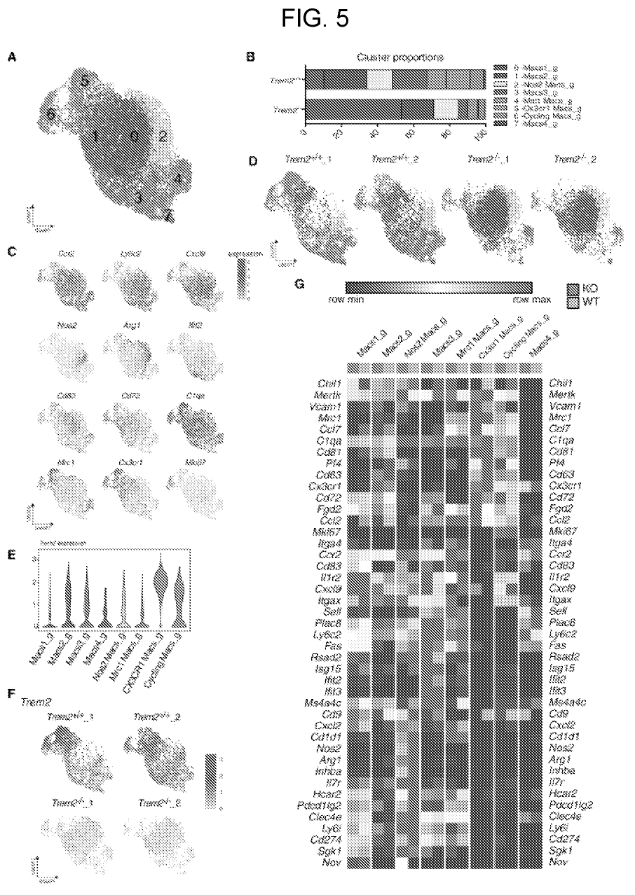

To delve more deeply into the influence of TREM2 on macrophage subsets, we further re-clustered macrophages into eight subsets (FIG. 5A-FIG. 5D), all of which expressed TREM2 (FIG. 5E and FIG. 5F). We confirmed that CX₃CR1-Macs-g, Mrc1-Macs-g, Cycling-Macs-g, and Macs3-g were poorly represented in Trem2^{-/-} MCA tumors. Genes that have been associated with immunosuppression in MCA, such as CX₃CR1, Mrc1, Mertk, and CD81, were expressed by either CX₃CR1- or Mrc1-Macs-g (FIG. 5B-FIG. 5D and FIG. 5G) (Gubin et al., 2018). Macs-3-g exhibited a profile reminiscent of the IFNα-β signature (Rsad2, Isg15, Ifit2, and Ifit3). Macs1-g and Macs2-g expressed monocyte markers such as Ccr2 and ll1r2, suggesting that they may encompass macrophages recently immigrated from blood. The Macs2-g subset, expressing C1qa and Cd72, was slightly diminished in Trem2^{-/-} MCA tumors. On the other hand, the Macs1-g cluster, which expressed genes previously associated with IFN-y imprinting and immunostimulation (Cxcl9 and Cd83) was much more predominant in Trem2^{-/-} MCA tumors. Nos2-Macs-g (Nos2 and Arg1) and Macs4-g (Itga4) were not altered (FIG. 5B-FIG. 5D and FIG. 5G). Interestingly, TREM2 expression was particularly high in two of the clusters poorly represented in Trem2^{-/-} MCA tumors, CX₃CR1-Macs-g and Cycling-Macs-g, suggesting a correlation between the level of TREM2 expression and dependence on TREM2 for sustaining cell numbers. We also re-clustered lymphoid cells, revealing heightened expression of activation markers, such as IFNy and PD-1 (Pdcd1) by T cells and NK cells in Trem2^{-/-} MCA tumors (FIG. 4F-FIG. 41).

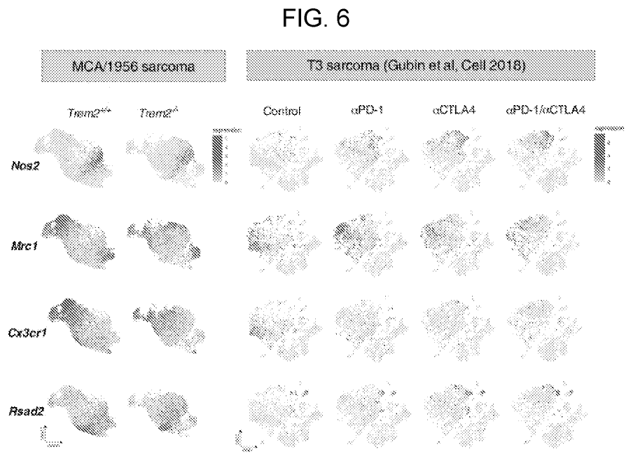

Finally, we compared the gene signatures of the eight macrophage clusters identified within MCA tumors with those reported for the intratumoral macrophage compartment in a non-immunogenic MCA tumor related to ours, which, although on a 129 background, was treated with immune-checkpoint therapy (ICT) (Gubin et al., 2018). Mrc1 and CX₃CR1 cluster reductions observed in Trem2^{-/-} mice were consistent with similar reductions induced by ICT. In contrast, Nos2⁺ and Rsad2⁺ macrophages were only increased in ICT-treated but not in Trem2^{-/-} mice (FIG. 6). We conclude that TREM2 deficiency drives a complex remodeling of macrophage infiltrate that promotes enrichment and activation of T cells and NK cells. Such reshaping resembles, only in part, that which was previously observed following ICT.

### TREM2 Deficiency Enhances Checkpoint Blockade

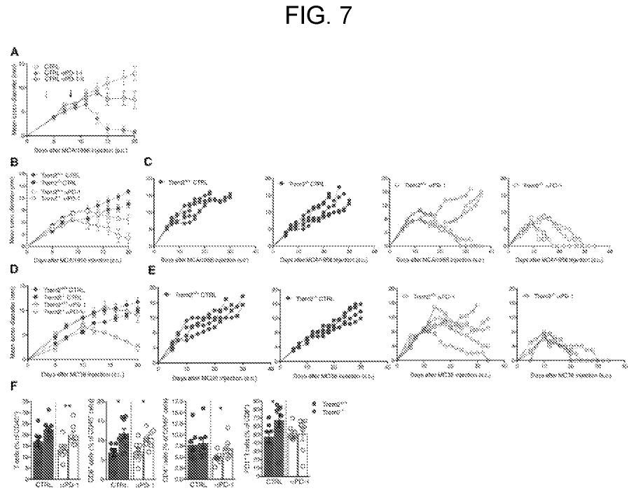

The observed resistance of Trem2^{-/-} mice to tumor growth prompted us to determine whether TREM2 deficiency can enhance antitumor responses unleashed by checkpoint blockade. Although the MCA/1956 sarcoma grows progressively in immunocompetent hosts, it retains sufficient immunogenicity to be effectively controlled by checkpoint immunotherapy with anti-PD-1 (Li et al., 2018). Indeed, when anti-PD-1 treatment was initiated early (on day 3 after injection of tumor cells), MCA/1956 was rejected in 100% mice (FIG. 7A). To determine whether lack of TREM2 can enhance anti-PD-1 immunotherapy, we chose a sub-optimal scheme for PD-1 administration, starting treatment at a late time point (on day 8 after injection of tumor cells) (Li et al., 2018). Following this treatment protocol, only 40% of WT mice rejected the tumor, whereas 100% of Trem2^{-/-} mice did (FIG. 7B and FIG. 7C). Since MC38 is also responsive to checkpoint blockade (Harjunpää et al., 2018; Li et al., 2018; Woo et al., 2012), we examined whether lack of Trem2 could enhance suboptimal anti-PD-1 treatment of this tumor. Indeed, all Trem2^{-/-} mice controlled tumor growth as opposed to only a few WT mice (FIG. 7D and FIG. 7E).

We next analyzed the immune infiltrate 14 days after tumor injection to define T cell responses. Trem2^{-/-} mice showed a trend in the increase of intratumoral αβ T cells, which was significant in anti-PD-1 treated mice (FIG. 7F). CD8⁺ T cells were increased in Trem2^{-/-} mice with or without anti-PD-1, as were PD-1⁺ CD8 T cells, corroborating our initial results (see FIG. 2C). PD-1⁺ CD4 T cells were only increased in Trem2^{-/-} mice treated with anti-PD-1 (FIG. 7F). Treg, B, NK, and Lag3⁺ T cell proportions did not change in the absence of TREM2 (data not shown). Altogether, these results demonstrate that TREM2 deficiency augments the efficacy of ICT in two transplantable tumor models responsive to anti-PD-1. Enhancement of ICT was associated with augmented T cell infiltration and activation, at least in the MCA model.

### Anti-TREM2 Treatment Is Protective in the Sarcoma Model

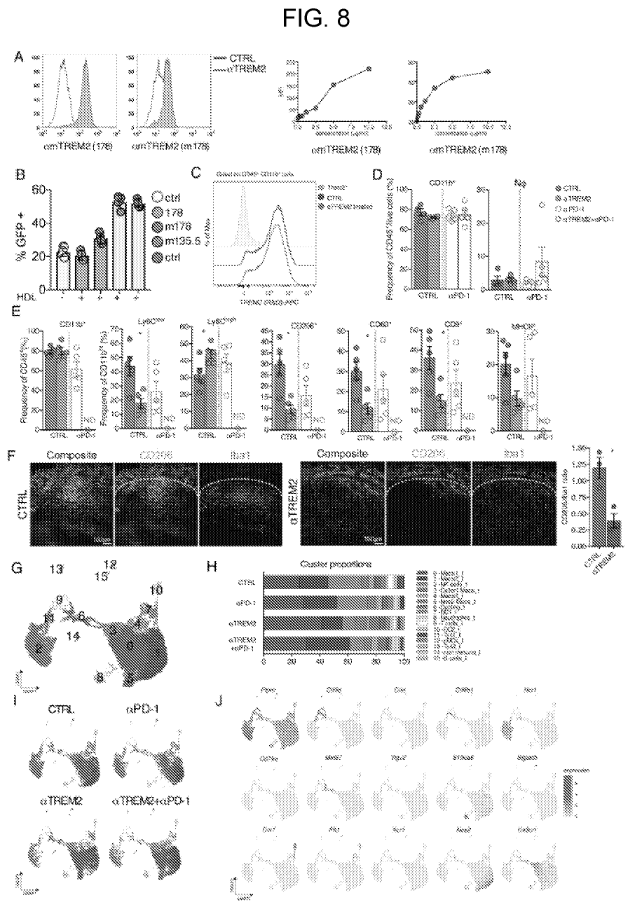

Since genetic deletion of Trem2 is associated with reduced tumor growth and enhanced response to checkpoint blockade, we sought to test the therapeutic potential of mAb modulation of TREM2. We previously showed that mAb 178 is specific for mouse TREM2 (Turnbull et al., 2006). This mAb was originally established using a standard hybridoma approach by immunizing rats with the recombinant ectodomain of TREM2. For tumor immunotherapy, we generated a recombinant form of mAb 178, in which the variable region of the heavy chain was grafted onto a mouse IgG2a constant region backbone that had been mutated in the Fc domain (LALAPG) to prevent recognition by Fc receptors and complement and the consequent induction of antibody-dependent cellular cytotoxicity or antibody-dependent phagocytosis. Both native and Fc mutated anti-TREM2 antibodies specifically stained TREM2 transfected cells in a dose-dependent manner (FIG. 8A). To see whether mAb 178 modulates TREM2, we tested the Ca²⁺-driven reporter cell line 2B4, stably transfected with TREM2 together with DAP12 (FIG. 8B) (Wang et al., 2016). In these reporter cells, engagement of TREM2 promotes Ca²⁺ signals that lead to nuclear translocation of NFAT and NFAT-driven synthesis of GFP, which is detected by flow cytometry. Lipidated HDL, a well-described TREM2 ligand (Song et al., 2017), induced GFP in TREM2 reporter cells. Both native and Fc mutated mAb 178 inhibited HDL-induced GFP expression, indicating that mAb 178 blocks ligand binding to TREM2 (FIG. 8B). To exclude in vivo depleting activity of recombinant mAb 178, we assessed its impact on TREM2⁺ thioglycollate-induced peritoneal macrophages after intraperitoneal (i.p.) injection (FIG. 8C). To evaluate TREM2⁺ macrophage numbers, we used an antibody recognizing a different epitope from that bound by mAb 178. The representation of TREM2⁺ macrophages was not affected by mAb 178 (FIG. 8C), indicating no antibody-mediated depletion, consistent with lack of effector function in the mutated Fc fragment.

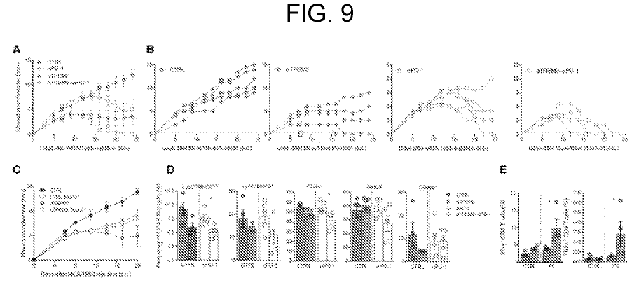

We then tested the recombinant anti-TREM2 in vivo in the MCA/1956 model with or without anti-PD-1. As control, we used an Fc mutated recombinant mAb specific for human ILT1, a receptor not encoded in mice. Administration of anti-TREM2 was initiated at day 2 after tumor injection and was repeated every 5 days until the end of the experiment. Anti-PD-1 was given following the suboptimal scheme described above (FIG. 7A). Treatment with anti-TREM2 alone afforded significant but incomplete control of tumor growth (FIG. 9A and FIG. 9B). However, the combination of anti-TREM2 and suboptimal anti-PD-1 conferred complete tumor control in all mice tested (FIG. 9A and FIG. 9B). We conclude that anti-TREM2 mAb 178 has anti-tumor activity and augments anti-PD-1 checkpoint blockade. Interestingly, tumor growth was more attenuated in anti-TREM2-treated mice than in Trem2^{-/-} mice (FIG. 9C). Yet, treatment of Trem2^{-/-} mice with the anti-TREM2 mAb did not further attenuate tumor growth, demonstrating that anti-TREM2 treatment has no off-target effects.

We next characterized the influence of anti-TREM2 on the tumor-immune infiltrate by flow cytometry. At an early time point (10 days after tumor injection), combined blockade of PD-1 and TREM2 was associated with diminished Ly6C⁺MHC∥⁻ and CD64⁺ subsets within the myeloid compartment. Neither anti-PD-1 nor anti-TREM2 treatment alone promoted significant changes (FIG. 9D and FIG. 8D). At a later time point (24 days after tumor injection), in parallel with a considerable shrinkage of the tumor volume, anti-TREM2 alone showed a significant impact on the myeloid compartment: while the total number of macrophages was unchanged, the representation of Ly6C^{low}MHC∥⁻ cells, along with subsets expressing CD206, CD63, and CD9, was reduced (FIG. 8E). Immunofluorescence analyses of tumor sections confirmed decreased CD206 expression within the tumor myeloid infiltrate (identified by staining for the myeloid marker Iba1) (FIG. 8F). Mice treated with suboptimal anti-PD-1 evinced no obvious changes, likely due to the short duration of the treatment. Since mice treated with both anti-TREM2 and anti-PD-1 had entirely cleared the tumors, no infiltrate could be analyzed. Anti-TREM2 had no obvious impact on the representation of lymphoid subsets at early or late time points (data not shown). However, analysis of T cell cytokine production ex vivo indicated that anti-TREM2 treatment augments IFNy and TNFα production by intra-tumoral CD8⁺ and CD4⁺ T cells, respectively (FIG. 9E). Altogether, these data suggest that mAb-mediated modulation of TREM2 drives the remodeling of the intra-tumor myeloid compartment in a manner that promotes a protective T cell response and enhances anti-PD-1 immunotherapy.

### Anti-TREM2 Treatment Reshapes the Intratumoral Macrophage Infiltrate

To define the impact of anti-TREM2 mAb on the immune populations at high resolution, we resorted to scRNA-seq. We assessed four treatment conditions: control antibody, anti-TREM2 plus control antibody, suboptimal anti-PD-1 plus control antibody, and anti-TREM2 plus suboptimal anti-PD-1. Two biological replicates for each condition were examined. scRNA-seq was performed on CD45⁺ cells sorted from MCA/1956 tumors and analysis was performed 10 days after injection of the tumor. We obtained data for an average of 2,726 cells per sample with a coverage of 14,546 UMIs per cell. Unsupervised clustering by UMAP identified 16 clusters (FIG. 8G-FIG. 8J). Ptprc (CD45) was expressed in all clusters, with the exception of few non-immune cells (cluster 14). Cluster 9 expressed T cell markers (Cd3d, Cd4, and Cd8b1), while cluster 2 encompassed NK cells (Ncr1). Clusters 11 and 13 represented two small clusters of yδ⁺ T cells (Trgv2⁺). Cluster 15 (Cd79a), cluster 8 (S100a8), and cluster 12 (SiglecH) identified B cells, neutrophils, and pDCs, respectively. Cluster 6 corresponded to Mki67+ proliferating cells, including both T cells and CX₃CR1⁺ macrophages. Cluster 10 included DC2-like cells based on the expression of Flt3, Ccr7, Ccl22, and Ccl17, while cluster 7 represented Xcr1⁺ DC1-like cells (FIG. 8G-FIG. 8J). Macrophage subsets were grouped in clusters 0, 1, 3, 4, and 5 (FIG. 8G-FIG. 8J). While most clusters seemed similarly represented under all conditions, some of the macrophage clusters varied markedly, most notably the exclusive presence of cluster 5 in the anti-TREM2-treated tumor (FIG. 8G and FIG. 8J). No obvious changes were detected in the lymphoid compartment at this early time point.

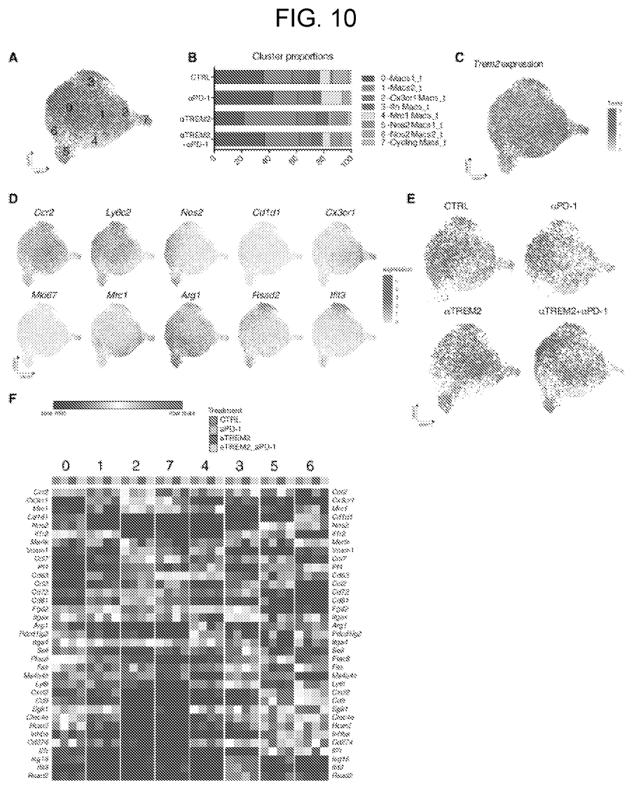

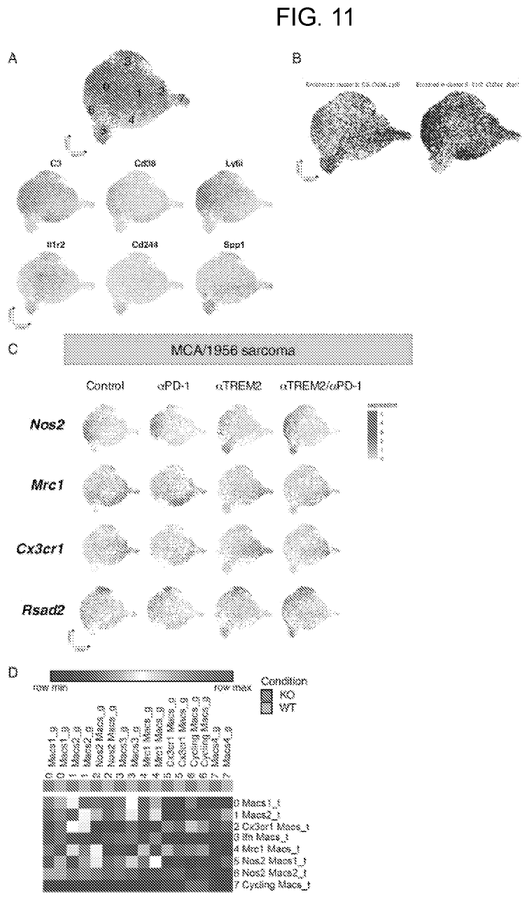

We then separately re-clustered macrophages to obtain deeper profiling (FIG. 10A and FIG. 10B). As in FIG. 5, macrophage subsets were designated with either a gene name or a number, depending on whether they expressed a typical macrophage gene or a constellation of genes. Clusters were further designated with a “t” to indicate that they were distinguished following antibody treatments. We identified eight subsets that broadly expressed TREM2 (FIG. 10A-FIG. 10E). CX₃CR1-Macs-t resided primarily in cluster 2, as well in Cycling Macs-t (cluster 7). Most Mrc1-Macs-t were confined to cluster 4, with few Mrc1⁺ cells in clusters 2 and 7. Nos2-Macs-t were present in clusters 5 and 6 (FIG. 10D and FIG. 10F), which were distinguished based on selective expression of II1r2, Cd244, and Spp1 (cluster 5), as well as C3, CD38, and Ly6i (cluster 6) (FIG. 11A and FIG. 11B). Cluster 3 exhibited a clear IFN signature, indicated by an enrichment for Isg15, Ifit3, and Rsad2 transcripts (FIG. 10D and FIG. 10F). Macs1-t (cluster 0) and Macs2-t (cluster 1) encompassed cells expressing monocyte markers such as Ccr2 and IL1r2 and hence may be monocytes that recently immigrated into the tumor from blood. The distinction into two subsets was marked by Ly6i (Macs1-t) and Fgd2 (Macs2-t) (FIG. 10D and FIG. 10F).

Some of the macrophage clusters were differentially represented after the distinct treatment regimens. Anti-TREM2 treatment, with or without anti-PD-1, induced de novo appearance of Nos2-Macs-t (cluster 5), which was virtually absent under other conditions (FIG. 10E). The population of Mrc1-Macs-t contracted following treatment with anti-TREM2. One of the macrophage subsets that recently immigrated into the tumor, Macs1-t, also declined, while the other one—Macs2-t—expanded, perhaps reflecting changes in the intratumoral differentiation of blood-derived monocytes. Cycling-Macs-t (which were also CX₃CR1⁺) were almost ablated by anti-TREM2 in combination with anti-PD-1 (FIG. 10E and FIG. 10F). We conclude that anti-TREM2 treatment induces a complex remodeling of the myeloid compartment that drives the appearance of Nos2-Macs-t and a parallel decline of Mrc1-Macs-t, Cycling-Macs-t, and Macs1-t recent immigrants.

Given that previous analysis of ICT-treated MCA tumors showed an increase of Nos2- and Rsad2-expressing macrophages and a reduction of Mrc1-and CX₃CR1-expressing macrophages (FIG. 6) (Gubin et al., 2018), we examined the expression of the same genes in anti-TREM2-treated tumors (FIG. 11C). Nos2⁺ cells were expanded and Mrc1⁺ cells were reduced in anti-TREM2 and anti-TREM2 plus anti-PD-1-treated MCA, in line with ICT-treated tumors. In contrast, Rsad2⁺ Macs were only increased following ICT and anti-TREM2 plus anti-PD-1, but not after anti-TREM2 treatment alone (FIG. 11C). We further compared the gene signatures of macrophage clusters identified in Trem2^{+/+} and Trem2^{-/-} tumor-bearing mice with those found in mice treated with blocking antibodies (FIG. 11D). Results are represented in a heatmap showing the magnitude of difference or similarity of each cluster in the two experiments. CX₃CR1 Macs_g, which declined in the absence of TREM2, overlapped with Mrc1 Macs_t, which comparably declined in anti-TREM2-treated mice, as well as with CX₃CR1 Macs_t. Similarly, Nos2 Macs_g overlapped with the Nos2 Macs1_t and Nos2 Macs2_t clusters in anti-TREM2-treated mice. Finally, cycling Macs_g corresponded to cycling Macs_t, which were diminished in mice treated with combined anti-TREM2 and anti-PD-1. Altogether, TREM2 deficiency, and anti-TREM2 and anti-ICT treatments, induced some consistent changes in tumor-associated macrophages.

### Trem2 Is Selectively Expressed in Human Tumor Macrophages

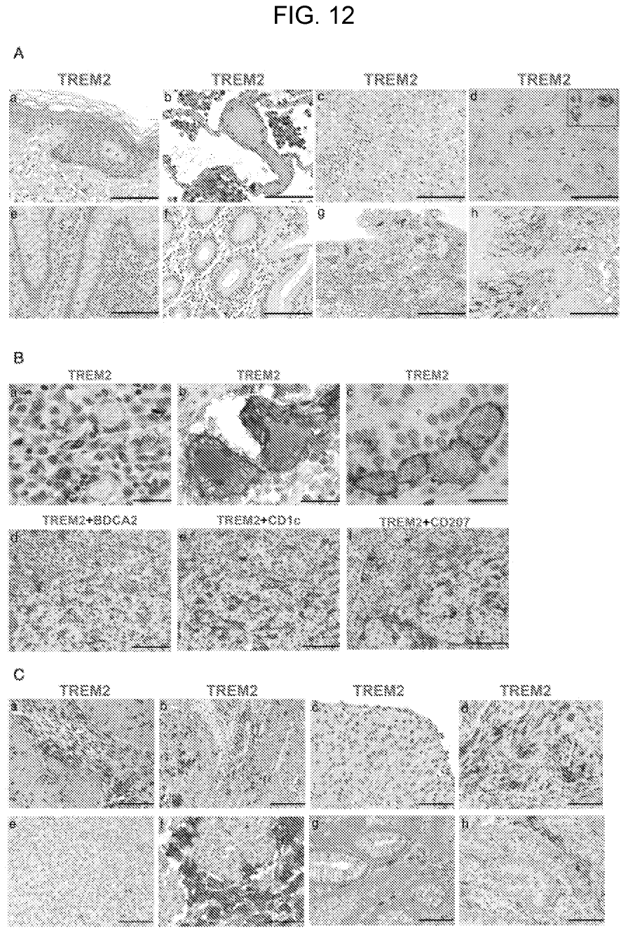

To address the significance of our findings in human tumors, we first analyzed TREM2 protein expression in macrophages from human normal and tumor specimens by IHC. TREM2 expression was not detected in a large majority of macrophages in peripheral tissues, with the exception of microglia, MITF⁺ placental macrophages, macrophages of the endometrium, and alveolar macrophages (FIG. 12A). In contrast, TREM2⁺ macrophages were observed in 75% of carcinomas from various primary sites as assessed by screening of a multi-carcinoma tissue microarray containing 126 samples (data not shown). To circumvent potential tumor heterogeneity, we further extended this analysis to whole tissue sections obtained from 99 tumor samples, which included carcinomas from many sites. We confirmed the presence of TREM2⁺ macrophages in virtually all cases (representative examples are shown in FIG. 13A). Morphology of TREM2⁺ tumor macrophages varied from small monocytoid to large foamy or multinucleated giant cells, with TREM2 expression mainly localized on the cell membrane (FIG. 13B and FIG. 12B). TREM2⁺ tumor macrophages co-expressed CD68, CD163, CSF1R, and nuclear MAFB; foamy TREM2⁺ tumor macrophages also co-expressed MITF, a transcription factor involved in lysosomal biogenesis that marks a regulatory myeloid subset of human placenta (Costa et al., 2017). No TREM2 expression was observed in BDCA2⁺ plasmacytoid DC, CD1c myeloid DC, or CD207 Langerhans cells (FIG. 12B).

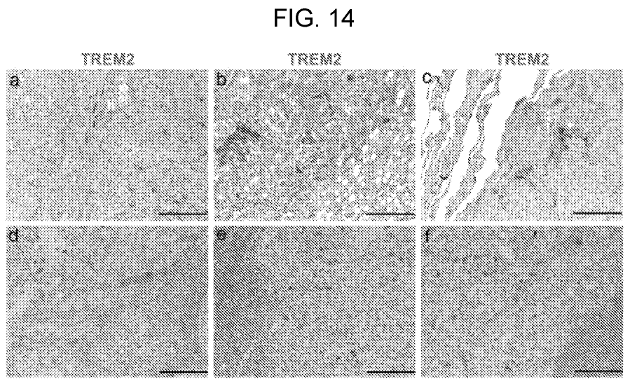

TREM2⁺ macrophages were scant or absent in benign or low-grade neoplastic lesions, as observed for colon adenomas, low grade papillary urothelial carcinomas, and skin nevi (FIG. 12C). Because of the clinical relevance of systemic dissemination of cancer cells, we tested TREM2 expression in macrophages from nodal (n = 33) and distant metastasis (n = 12). We found TREM2⁺ macrophages surrounding and infiltrating metastatic nests in tumor-draining lymph nodes, as well as in distant liver and lung metastasis (FIG. 14). Altogether, TREM2 emerged as a marker of tumor-infiltrating macrophages in all cancers. One striking finding was the selective intratumor localization of TREM2⁺ macrophages as opposed to TREM2⁻ macrophages, which were found in the surrounding stromal area. Since TREM2 expression is induced by CSF-1, GM-CSF, IL-4, and IL-13 (Cella et al., 2003), the expression of TREM2 may reflect intratumoral production of these cytokines.

### TREM2 Correlates With Poor Disease Prognosis

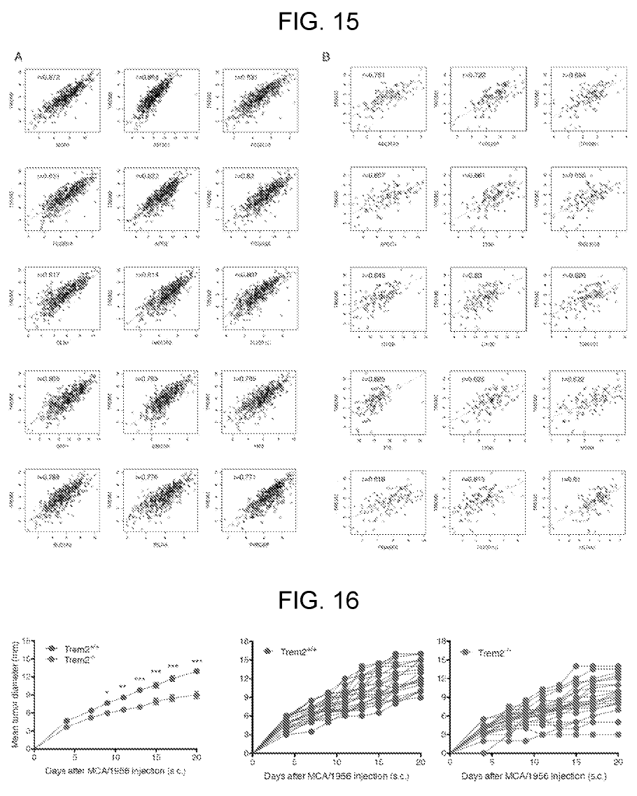

We next explored The Cancer Genome Atlas (TCGA) database for correlations between TREM2 expression and clinical outcome in tumor types related to our experimental models. With a threshold of 75% quantile, TREM2 expression correlated with worse overall survival in the CRC cohort (FIG. 13C). In the TNBC cohort, TREM2 expression correlated with worse overall and relapse-free survival using the 75% quantile as cutoff (FIG. 13D). Similar results were obtained using the TREM2 median expression. Analysis of the TCGA database also revealed that TREM2 expression correlated with that of myeloid signature genes in both CRC and TNBC cohorts. In CRC patients, the top genes correlating with TREM2 included the scavenger receptor MSR1, the Fc receptor FCGR1A, the TREM2 adaptor protein TYROBP, the exhaustion marker HAVCR2, and several lipid metabolism genes (APOC1, APOE, and OLR1) previously associated with lipid-associated macrophage signature (FIG. 15A) (Biswas and Mantovani, 2012; Cochain et al., 2018; Jaitin et al., 2019). Consistently, in TNBC patients, TREM2 highly correlated with MSR1, FCGR1C, TYROBP, monocyte-macrophage receptors (SIGLEC8 and LILRA2), genes associated with macrophage activation and phagocytosis (LY86, LY96, and RNASE6), macrophage metabolism (APOC1, ADORA3, TBXAS1, and FTL), and complement activation (C1QB and C1QC) (FIG. 15B). Interestingly, in a multivariate survival analysis of TNBC, TREM2 had additional survival prognostic ability independent of other myeloid genes (TABLE 1), suggesting a direct contribution to pathogenesis. Other types of human tumors also showed a correlation between TREM2 and macrophage signature genes, but the association of TREM2 expression with patient overall or relapse-free survival was not significant (data not shown), suggesting that TREM2’s impact on tumor-infiltrating macrophages and tumor immune responses might be context specific.

### Discussion

This study demonstrates that constitutive lack of TREM2 or anti-TREM2 treatment curbs tumor growth and leads to complete tumor regression when associated with suboptimal PD-1 immunotherapy. mAb inhibiting CTLA-4 or PD-1 have been extensively shown to unleash T cell effector functions to control tumors in both mice and some cancer patients (Sharma and Allison, 2015; Topalian et al., 2015). However, checkpoint blockade is incompletely effective for certain tumors, because they can escape using multiple mechanisms, one of which is the generation of a tumor microenvironment rich in myeloid cells with a strong immunosuppressive function. Thus, efforts are currently ongoing to complement checkpoint blockade with treatments targeting myeloid cells (Mantovani et al., 2017). Approaches have been developed to deplete myeloid cells from tumors, block their pro-tumoral functions, or restore their immunostimulatory properties. Among others, these strategies include inhibition of colony stimulating factor 1 receptor (CSF-1R) (Hume and MacDonald, 2012; Ries et al., 2014), CD24-Siglec10, CD47-SIRPα signaling (Barkal et al., 2019; Iribarren et al., 2018; Veillette and Chen, 2018; Willingham et al., 2012), and CXCR4-CXCL12 signaling (Hughes et al., 2015), as well as metabolic modulation, pharmacological modulation, and immunostimulation via an anti-CD40 agonistic antibody (Mantovani et al., 2017; Schoenberger et al., 1998; Wiehagen et al., 2017). The anti-TREM2 treatment presented here provides a novel therapeutic approach that broadens the armamentarium for myeloid cell targeting in tumors.

Analyses of the TCGA database suggested that anti-TREM2 treatment may be particularly promising in CRC and TNBC, because TREM2 expression inversely correlated with overall survival and relapse in these cancer patients. Beyond these correlations, our extensive pathology study of human tumors suggests that TREM2 may be a particularly attractive therapeutic target, as it was highly expressed in the vast majority of over 200 cases of primary and metastatic tumors that we examined by IHC, while it was poorly expressed in normal tissues. Therefore, TREM2 targeting may be widely applicable. We have previously shown that TREM2 is induced in human monocytes and mouse bone marrow cells upon exposure to GM-CSF and CSF-1 (Bouchon et al., 2001; Turnbull et al., 2006). Thus, we speculate that the high level of TREM2 expression in tumors reflects the production of these cytokines by several cell types in the tumor microenvironment, such as fibroblasts and myeloid cells. It is possible that the efficacy of anti-CSF-1 treatment previously observed in tumor models is partly due to the reduced expression of TREM2.

High-resolution analysis of the tumor cell infiltrate in the MCA model has revealed complex remodeling of the myeloid cell landscape in Trem2^{-/-} and anti-TREM2 treated mice. This transformation is epitomized by a consistent decline in macrophage populations expressing CX₃CR1 and MRC1, which is paralleled by the appearance of new macrophage clusters that express a set of activation markers in Trem2^{-/-} mice, or iNOS in anti-TREM2-treated mice. These changes partially overlapped with those reported in a model of MCA-induced sarcoma treated with optimal anti-PD-1 (Gubin et al., 2018). It is worth noting that the changes induced by TREM2 targeting do not fit the M2/M1 paradigm. Diminished macrophage subsets expressed genes encoding molecules involved not only in immunosuppression, such as MRC1 and MERTK, but also in regulation of lipid metabolism, fibrosis, survival, and proliferation. The macrophages that expanded in Trem2^{-/-} mice expressed CXCL9, which may reflect some exposure to IFN-y, but they did not evince a clear IFN-y-induced gene signature comparable to the classical M1 signature. Moreover, the macrophage subset inflated in anti-TREM2-treated mice expressed iNOS, which has been associated with immunostimulation (Hibbs et al., 1987; Murray et al., 2014; Stuehr and Nathan, 1989), as well as immunosuppression (Shi et al., 2001). Even combining anti-TREM2 with anti-PD-1 failed to elicit a clear M1 signature, perhaps due to the suboptimal anti-PD-1 regimen used in our experiments, which was initiated late after tumor cell injection. The observed changes in tumor macrophage infiltrates enhanced T-cell-mediated control of tumor growth, although we noted some variability in the extent of T cell expansion and effector function when comparing Trem2^{-/-}- and anti-TREM2-treated mice. It will be important to validate which key genes showcased in the transcriptional signatures of macrophages before and after TREM2 targeting stimulate or suppress T cell responses and how they operate.

The mechanisms by which TREM2 deficiency and anti-TREM2 treatment impact tumor macrophages remain unclear. We have previously shown that TREM2 cooperates with CSF-1 in sustaining macrophage proliferation and survival (Ulland et al., 2017). We noticed that two clusters diminished in Trem2^{-/-} MCA express the highest levels of TREM2. Thus, it is possible that lack or blockade of TREM2 binding with endogenous ligands selectively affects the survival of certain macrophage subsets, allowing other subsets to expand. TREM2 modulation may also improve the antigen-presenting function of tumor macrophages, since Trem2^{-/-} macrophages were shown to present antigens to T cells more effectively than WT macrophages in vitro (Ito and Hamerman, 2012). Interestingly, we noticed that anti-TREM2 treatment was more effective than TREM2 deficiency in controlling tumor size. While antibody treatment affects tumor-associated macrophages acutely, constitutive TREM2-deficiency might allow direct and/or indirect compensatory responses of myeloid cells that impact tumor growth. For example, constitutive lack of TREM2 may free up more DAP12 for other DAP12-associated receptors in macrophages, increasing their signaling and pro-survival capabilities. Additionally, anti-TREM2 may effectively interfere with some, but not all, TREM2-signaling pathways, which include not only proliferation and survival, but also metabolism, migration, and chemokine production (Peng et al., 2010; Ulland et al., 2017). Finally, anti-TREM2 may actually over-activate rather than block TREM2-controlled pathways, leading to anergy and/or exhaustion. Future biochemical experiments will be important to characterize how anti-TREM2 interferes with ligand binding as well as signaling pathways.

Key Resources Table

| REAGENT or RESOURCE | SOURCE | IDENTIFIER |
|---|---|---|
| Antibodies | | |
| hTREM2 (rabbit, clone D8I4C) | Cell Signaling Technology | Cat #91068 |
| hCD163 (mouse, clone 10D6) | Thermo Scientific | Cat #MS-1103-S0 |
| hCD68 (mouse, clone PG-M1) | Dako | Cat #M0876 |
| hMITF (mouse, clone D5, 1:50) | Dako | Cat #M3621 |
| hCD1c (mouse, clone OTI2F4) | Abcam | Cat #ab156708 |
| hCD207 (mouse, clone 12D6) | Leica | Cat# NCL-L-LANGERIN |
| hMAFB (polyclonal rabbit) | Sigma-Aldrich | Cat #HPA005653 |
| hCSF-1R (rabbit, clone FER216) | Merck-Millipore | Cat #MABS1163 |
| mouse Iba1, (rabbit, clone E404W) | Cell Signalling Technology | Cat #17198S |
| mouse CD206-AlexaFluor488 (rat, clone C068C2) | Biolegend | Cat #141710 |
| anti-rabbit-AlexaFluor647 (donkey) | Invitrogen | Cat #A31573 |
| CD45-BV605 (clone 30-F11) | Biolegend | Cat #103139 |
| CD45-AlexaFluor700 (clone 30-F11) | Biolegend | Cat #103128 |
| CD11b-PerCPCy5.5 (clone M1/70) | Biolegend | Cat #101228 |
| CD11b-PECy7 (clone M1/70) | Biolegend | Cat #101216 |
| CD11b-BV421 (clone M1/70) | Biolegend | Cat #101235 |
| I-A/I-E-BV650 (clone M5/114.15.2) | Biolegend | Cat #107641 |
| Ly6C-BV421 (clone HK1.4) | Biolegend | Cat #128031 |
| Ly6C-APC (clone HK1.4) | Biolegend | Cat #128016 |
| Ly6C-PerCP (clone HK1.4) | Biolegend | Cat #128028 |
| Ly6C-PE (clone HK1.4) | eBioscience | Cat #12-5932-82 |
| Ly6G-AlexaFluor700 (clone 1A8) | Biolegend | Cat #127621 |
| Ly6G-APC (clone 1A8) | Biolegend | Cat #127614 |
| CD64-APC (clone X54-5/7.1) | Biolegend | Cat #139305 |
| CD64-BV605 (clone X54-5/7.1) | Biolegend | Cat #139323 |
| B220-BUV395 (clone RA3-6B2) | BD Biosciences | Cat #563793 |
| B220-PerCPCy5.5 (clone RA3-6B2) | BD Biosciences | Cat #552771 |
| CD206-PECy7 (clone C068C2) | Biolegend | Cat #141719 |
| iNOS/Nos2-PE (clone CXNFT) | Thermo Fisher | Cat #12-5920-80 |
| CD9-PE (clone MZ3) | Biolegend | Cat #124805 |
| CD63-PerCPCy5.5 (clone NVG-2) | eBioscience | Cat #46-0631-80 |
| PD-L2-P E/Dazzle™ 594 (clone TY25) | Biolegend | Cat #107215 |
| TCRβ-PE (clone H57-597) | Biolegend | Cat #109208 |
| CD8a-BV785 (clone 53-6.7) | Biolegend | Cat #100749 |
| CD4-PE/Dazzle™ 594 (clone RM4-5) | Biolegend | Cat #100566 |
| PD1-FITC (clone J43) | Invitrogen | Cat #11-9985-82 |
| CD19-PacificBlue (clone 6D5) | Biolegend | Cat #115523 |
| FOXP3-AlexaFluor647 (clone MF-14) | Biolegend | Cat #126408 |
| NK1.1-BV650 (clone PK136) | Biolegend | Cat #108736 |
| LAG-3-PECy7 (clone C9B7W) | Biolegend | Cat #125225 |
| TCRyδ-FITC (clone eBioGL3) | eBioscience | Cat #11-5711-85 |
| IFNy-PE (clone XMG1.2) | Biolegend | Cat #505808 |
| TNFα-FITC (clone MP6-XT22) | Biolegend | Cat #506304 |
| TREM2-APC (clone 237920) | R&D | Cat #FAB17291A |
| Anti-human ILT1-Fc mutated (clone 135.5) | This paper | N/A |
| Anti-murine TREM2-Fc mutated (clone 178) | This paper | N/A |
| Anti-murine TREM2 (clone 178) | (Turnbull et al., 2006) | N/A |
| InVivoMab anti-mouse CD8α (53-6.7) | BioXCell | Cat #BE0004-1 |
| Anti-mouse CD4 (GK1.5) | In house | N/A |
| Rat IgG2a Isotype Control | Leinco Technologies, Inc. | Cat #1-1177 |
| InVivoMab rat IgG2a isotype control (LTF-2) | BioXCell | Cat #BE0090 |
| REAGENT or RESOURCE | SOURCE | IDENTIFIER |
| Bacterial and Virus Strains | | |
| 10-beta Comp. E. coli | New England Biolabs | Cat #C3019H |
| Surgical samples of non-neoplastic tissues (human skin, lung, liver, brain, colon, stomach, uterus and placenta) and neoplastic tissues (primary carcinomas, tumor draining lymph nodes and distant metastasis). | Tissue bank of the Department of Pathology (ASST, Spedali Civili di Brescia, Brescia, Italy) | N/A |
| Multi-tumour tissue microarrays (TMA) | (Vermi et al., 2014) | N/A |
| REAGENT or RESOURCE | SOURCE | IDENTIFIER |
| Chemicals, Peptides, and Recombinant Protiens | | |
| Collagenase from Clostridium histolyticum | Sigma | Cat #C5138-5G |
| Aqua fluorescent reactive dye | Life technologies | Cat #L34966 |
| Novolink Polymer. | Leica Microsistem | Cat #RE7200-CE |
| Mach 4 MR-AP (Biocare Medical), | Biocare Medical | Cat #M4U536 |
| Diamminobenzidine | Leica Microsistem | Cat #AR9432 |
| Ferangie-blue | Biocare Medical | Cat #FB813 |
| Prolong Diamong antifade mountant | Thermofisher | Cat #P36961 |
| HiTrap® Protein A High Performance | GE Healthcare | Cat #17-0403-01 |
| Xhol | New England Biolabs | Cat#R0146S |
| Bglll | New England Biolabs | Cat#R0144S |
| Nhel-HF | New England Biolabs | Cat #R3131S |
| Expi293™ Expression Medium | Thermo Scientific | Cat #A1435101 |
| Lipoprotein, high density from human plasma | Sigma | Cat # L8039-10MG |
| Thioglycollate medium | Sigma | Cat #T9032 |
| REAGENT or RESOURCE | SOURCE | IDENTIFIER |
| Critical Commercial Assays | | |
| FOXP3/Transcriptio n Factor Staining Buffer Set | Invitrogen (Thermo Fisher Scientific) | Cat #00-5523-00 |
| Endosafe LAL Cartridges | Charles River Laboratories, | Cat #PTS5505F |
| ExpiFectamine™ 293 Transfection Kit | Thermo Scientific | Cat#A14524 |
| EndoFree Plasmid Maxi Kit | QIAGEN | Cat #12362 |
| Gibson Assembly Master Mix | New England Biolabs | Cat #E2611S |
| Chromium™ Single Cell 3′ GEM, Library & Gel Bead Kit v3, 16 rxns | 10x Genomics | Cat #PN-1000075 |
| Chromium™ Chip B Single Cell Kit, 48 rxns | 10x Genomics | Cat #PN-1000073 |
| Chromium i7 Multiplex Kit | 10x Genomics | Cat #PN-120262 |
| dsDNA High Sensitivity Assay Kit | Invitrogen | Cat #Q32851 |
| REAGENT or RESOURCE | SOURCE | IDENTIFIER |
| Deposited Data | | |
| aTREM2 and TREM2 KO experiment with MCA sarcoma tumor, 10x single cell RNA-seq | this paper | GSE151710 |
| REAGENT or RESOURCE | SOURCE | IDENTIFIER |
| Experimental Models: Cell Lines | | |
| MCA/1956 | generated in house | N/A |
| MC38 | (Gilfillan et al., 2008) | N/A |
| PyMT | (Su et al., 2016) | N/A |
| Expi 293F | (Stadlbauer et al., 2019) | N/A |
| mTREM2-reporter cell line | (Wang et al., 2015) | N/A |
| REAGENT or RESOURCE | SOURCE | IDENTIFIER |
| Experimental Models: Organisms/Strains | | |
| C57BL6/J | Jackson | Cat #000664 |
| Trem2^{-/-} and Trem2^{+/+} mice (C57BL6/J) | In house | N/A |
| REAGENT or RESOURCE | SOURCE | IDENTIFIER |
| Oligonucleotides | | |
| VH and CH1 gBlock (clone: 178) | Integrated DNA technologies | N/A |
| VL and C_{K}gBlock (clone: 178) | Integrated DNA technologies | N/A |
| VH and CH1 gBlock (clone: 135.5) | Integrated DNA technologies | N/A |
| VL and C_{K}gBlock (clone: 135.5) | Integrated DNA technologies | N/A |
| REAGENT or RESOURCE | SOURCE | IDENTIFIER |
| Recombinant DNA | | |
| Anti-murine Trem2 antibody plasmids (clone: 178) | generated in house | GENEWIZ |
| Fc-mutant mlgG2A control antibody plasmids (clone: 135.5) | generated in house | GENEWIZ |
| REAGENT or RESOURCE | SOURCE | IDENTIFIER |
| Software and Algorithms | | |
| FlowJo v9.3 | FlowJo | www.flowjo.com |
| Prism v6 | GraphPad | www.graphpad.co m |
| Seurat | Butler et al., 2018 | N/A |
| Cellranger | https://support.10xgenomics.com/singl e-cell-gene-expression/software/downloads/latest | N/A |
| Phantasus | https://artyomovlab.wustl.edu/phantas us/ | N/A |
| ggplot2 | (Wickham, 2016) | N/A |
| R | R Core Team (2017). R: A language and environment for statistical computing. R Foundation for Statistical Computing, Vienna, Austria. URL https://www.R-project.org/. | N/A |

### Data and Code Availability

The accession number for the data reported in this paper is GEO:GSE151710.

### Experimental Model and Subject Details

### Animals

Mice were of mixed sexes. Mice within experiments were age and sex matched. Mice were housed under specific pathogen free conditions. Mice from different genotypes were cohoused from birth and separated during the experiment (the day of tumor injection). Mice did not undergo any procedures prior to their stated use. Mice used in this study include WT C57BL/6J and Trem2^{-/-} animals bred at Washington University School of Medicine animal facility. For experiments with wild-type groups only (e.g., experiments with anti-TREM2 treatment), wild-type mice were purchased from Jackson. All animals were backcrossed until at least > 98% C57BL/6J confirmed by genotype wide microsatellite typing. For tumor models, animals were injected at 8 weeks of age for MCA/1956 and MC38 cell lines. For the PyMT model, female mice were injected at 10 weeks of age. All studies performed on mice were done in accordance with the Institutional Animal Care and Use Committee at Washington University in St. Louis approved all protocols used in this study.

### Tumor Models

MCA/1956 or MC38 cells were washed and resuspended in PBS and injected subcutaneously (10⁶ cells/mouse in 100 µl PBS). Mice were previously shaved on the flank. 10⁵ PyMT cells/mouse were suspended in 50 µl of sterile PBS and Matrigel Matrix (Corning #354234) and injected into the mammary fat pad (MFP). Mice were monitored every day and tumors were measured by caliper every other day. Mice were sacrificed at day 10, at day 24 or when tumors reached 1.5 cm of diameter.

### In Vivo Treatments

Mice were treated intraperitoneally (i.p.) with anti-PD1 antibody (200 µg/mouse) every 3 days, starting at day 3 or day 8 after tumor injection, as specified. Mice were treated i.p. with anti-TREM2 antibody (clone 178; 200 µg/mouse) every 5 days, starting at day 2 after tumor injection. Anti-hILT1 (clone 135.5) was used as a control. Anti-CD4 and anti-CD8 treatments were started one day before tumor injection (200 µg/mouse) and then administrated every 3 days for the entire duration of the experiment.

To isolate peritoneal macrophages, mice were treated with anti-TREM2 (clone 178) or control antibodies prior to 5% thioglycollate. We collected peritoneal cells after 72 h and stained them with the commercial antibody anti-TREM2 (clone 237920, R&D) in combination with antibodies specific for myeloid markers.

### TREM2 Blocking in Vitro Experiment

TREM2 reporter cell lines expressing GFP upon TREM2 engagement were produced in our laboratory and previously described (Wang et al., 2015). HDLs (100 µg/mL; SIGMA) were immobilized on plates. Reporter cells were added and cultured overnight in the presence of antibodies (20 µg/mL) in complete medium. Native (rat IgG2a) and recombinant Fc mutated (mouse IgG2a) anti-murine TREM2 antibodies (clone 178) were used. Recombinant Fc mutated (mouse IgG2a) anti-human ILT1 antibody (clone 135.5) was used as a control.

### Human Tissues

Formalin-fixed paraffin-embedded tissue blocks used for this study were retrieved from the tissue bank of the Department of Pathology (ASST, Spedali Civili di Brescia, Brescia, Italy). Non-neoplastic tissues included human skin, lung, liver, brain, colon, stomach, uterus, and placenta. Neoplastic tissues included multi-tumor TMA (Vermi et al., 2014), a set of primary carcinomas (n = 99), tumor-draining lymph nodes (n = 33) and distant metastasis (n = 12). Primary carcinomas from various sites are as follows: breast n = 14; ovary n = 10; skin = 5; lung n = 10; stomach n = 10; colon n = 10; pancreas n = 5; liver n = 4; kidney n = 7; bladder n = 10; gliomas n = 5; lymphomas n = 4 and melanomas n = 5.

### Method Details

### Flow Cytometry

Single cell suspensions were prepared from tumors upon sacrifice. Tumors were minced and digested with Collagenase IV (Sigma) for 30 min at 37° C. Cells were filtered through 70-µm strainers, washed with PBS and stained for flow cytometry. The following antibodies were used: CD45-BV605 or-AlexaFluor700 (clone 30-F11), CD11b-PerCPCy5.5 or -PECy7 or -APC or-BV421 (clone M1/70), I-A/I-E-BV650 (clone M5/114.15.2), Ly6C-BV421 or -APC or-PerCPCy5.5 or -PE (clone HK1.4), Ly6G -AlexaFluor700 or -APC (clone 1A8); CD64 -APC or-BV605 (clone X54-5/7.1), B220-BUV395 or-PerCPCy5.5 (clone RA3-6B2); CD206 -PeCy7 (clone C068C2); iNOS/Nos2 -PE (clone CXNFT); CD9-PE (clone MZ3); CD63-PerCPcy5.5 (clone NVG-2); PD-L2-PECF594 (clone TY25); TCRβ-PE (clone H57-597); CD8-BV785 (clone 53-6.7); CD4-PECF594 (clone RM4-5); PD-1-FITC (clone J43); FOXP3-APC (clone MF-14); CD19-PacificBlue (clone 6D5); NK1.1-BV650 (clone PK136); Lag3-PECy7 (clone C9B7W); TCRyδ-FITC (clone eBioGL3); IFNy-PE (clone XMG1.2); TNFα-FITC (clone MP6-XT22). TREM2 reporter cell line was stained with anti-TREM2 antibodies produced in our lab (clone 178). Anti-rat IgG-PE and anti-mouse IgG-PE (Southern Biotech) were used as secondary antibodies. Cells were incubated with Fc block prior to staining. Cell viability was determined by Aqua LIVE/Dead-405 nm staining (Invitrogen), negative cells were considered viable. Foxp3/Transcription Factor Staining Buffer Set (eBioscience) was used for intracellular staining. Cells were analyzed on BD X20 or BD FACSymphony (BD Bioscience). Aria II (BD Bioscience) was used for sorting. Data were analyzed with FlowJo software (Treestar).

### Anti-TREM2 Antibody Production

Anti-mouse Trem2 monoclonal antibody (mAb) was cloned as previously described (Turnbull et al., 2006). For the Fc mutated antibody, the heavy chain variable (VH), CH1 and the light chain gene were sequenced from our 178 hybridoma (Syd Labs). To generate recombinant antibody, the heavy chain gBlocks (VH and CH1 mlgG2A; Integrated DNA technologies) was cloned into the pFUSE-mlgG2A-Fc1 vector with L234A, L235A, P329G (LALA-PG) mutation (Lo et al., 2017), and the light chain gBlock (VL and C_{K}; Integrated DNA technologies) was cloned into the mlgG2A-deficient pFUSE-mlgG2A-Fc1 vector. Then, the heavy chain and light chain plasmids were co-transfected into Expi293F cells (Thermo Scientific) for expression at mass ratio 1:2. Until cell viability reduces below 50% (5-7 days), the supernatant media was collected and antibody was purified with protein A agarose (GE Healthcare) (Stadlbauer et al., 2019). Following 3 days PBS dialysis, final preps were concentrated to about 10 mg/mL and freezed to store in -80° C. The heavy chain and light chain plasmids of the irrelevant Fc mutant mlgG2A control mAb (clone: 135.5) was co-transfected and purified similarly. After purification, all the antibody preps were confirmed with < 1 EU/mL endotoxin levels (Charles River Endosafe cartridge technology).

### T Cell Stimulation

Single cell suspensions were prepared from tumors upon sacrifice and leukocytes were enriched with a Percoll™ (GE Healthcare) gradient. Obtained cells were cultured in complete RPMI with or without PMA (10⁻⁷ M, Sigma)-lonomycin (500 ng/mL, Sigma) and Protein Transport Inhibitor Cocktail (eBioscience, 500X) for 4 h. Cells were collected and stained for flow cytometry analysis.

### Immunofluorescence Staining and Confocal Imaging

Tumor masses were resected and fixed in 4% PFA at 4° C. overnight. Fixed specimens were then dehydrated in 30% sucrose solution and cut into 50um-thick sections at the cryostat. Staining on free-floating sections was performed. Cryosections were blocked 4 h in 5% BSA solution and stained for 48 h at 4° C. with rabbit anti-lba1 (Cell Signaling Technology clone E404W, dilution 1:500) and rat anti-CD206-Alexafluor488 (Biolegend clone C068C2, dilution 1:200). Secondary staining was performed at room temperature for 2 h with fluorochrome-conjugated antibody (Life Technology Goat anti-rabbit Alexa-647, dilution 1:1000) and DAPI (Sigma, dilution 1:4000). Sections were then mounted on Superfrost glass slides (Fisher Scientific) and embedded in ProLong Diamond anti-fade mounting media (Thermofisher). Confocal imaging was carried out using a Zeiss LSM880 airyscan confocal microscope, with a 40X/1.4 oil-immersion objective. Each image was acquired in z stack/tile-scan mode to cover an area of 1 mm², and 10 um of thickness. Percentage of staining covered area was calculated in ImageJ, after automated background thresholding.

### Single Cell RNAseq

Live CD45+ cells were sorted from processed tumors. Using the Chromium Single Cell 3′ Reagent Kit v3 User Guide, single cell suspensions were partitioned into nanoliter droplets called Gel-bead-in-Emulsions (GEMs) to achieve single cell resolution. The cDNA generated within each individual GEM is tagged with a common 16 nt 10x barcode and a 12 nt unique molecular barcode during the RT reaction. Purified cDNA was amplified for 11 cycles before being purified using SPRIselect beads. Samples were then run on a Agilent Bioanalyzer to determine cDNA concentration. 10 uL of purified cDNA was used to generate the Illumina library for sequencing. For sample preparation on the 10x Genomics platform, the Chromium Single Cell 3′ GEM, Library & Gel Bead Kit v3 16 rxns (PN-1000075), Chromium Chip B Single Cell Kit, 48 rxns (PN-1000073) and Chromium i7 Multiplex Kit (PN-120262) were used. The single cell libraries were then sequenced on the Illumina NovaSeq 6000 S4 200 cycle flow cell generating 28×98 reads. A median sequencing depth of 50,000 reads/cell was targeted for each sample.

### Immunohistochemistry

Four-micron thick tissue sections were used for immunohistochemical staining. Sections were incubated with anti-TREM2 antibody (clone D8I4C, 1:100, Cell Signaling Technology) and the reaction was revealed using Novolink Polymer (Leica Microsystem). For double staining, after completing the first immune reaction, the second was visualized using Mach 4 MR-AP (Biocare Medical), followed by Ferangi Blue. Finally, the slides were counterstained with Meyer’s Haematoxylin. For double stain, TREM2 was coupled with anti-CD163 (clone 10D6, mouse, 1:50, Thermo Scientific), anti-CD68 (clone PG-M1, 1:200, Dako), anti-MITF (clone D5, 1:50, Dako), anti-CD1c (clone OTI2F4, 1:300, Abcam), anti-CD207 (clone 12D6, 1:150, Leica), anti-MAFB (polyclonal rabbit, 1:400, Sigma) or anti-CSF-1R (clone FER216, 1:1500, Millipore).

### Quantification and Statistical Analysis

### Statistical Analysis

Data were shown as mean ± SEM Two-way ANOVA was used to model data generated from factorial design with the combination of 2 factors and two-way ANOVA for repeated-measures was used to model longitudinal tumor growth between treatments followed by post hoc comparisons on treatment difference at time points. Mann-Whitney U-test was used to compare two groups. Statistics were calculated with GraphPad Prism 6 (GraphPad Software).

### Statistics and Reproducibility

FIG. 2A and FIG. 2B: n = 25 (Trem2^{+/+}); n = 24 (Trem2^{-/-}); four pooled experiments out of ten performed. FIG. 2C: n = 8-15. FIG. 2C (first panel): two pooled experiments. FIG. 2C (T cells): three pooled experiments. FIG. 2E: n = 4; one experiment performed; FIG. 5: n = 2; one experiment performed. FIG. 7A: n = 5 (CTRL); n = 5 (αPD1); n = 4 (αPD1ll); one experiment performed. FIG. 7B and FIG. 7C: n = 5; three experiments performed. FIG. 7D and FIG. 7E: n = 4 (Trem2^{+/+} CTRL); n = 5 (Trem2^{-/-} CTRL; Trem2^{+/+} αPD1; Trem2^{-/-} αPD1); two experiments performed. FIG. 7F: n = 7-8; pool of two experiments. FIG. 9A and FIG. 9B: n = 5; three experiments performed. FIG. 9C: n = 4 (CTRL; CTRL Trem2^{-/-}; αTREM2 Trem2^{-/-}); n = 5 (αTREM2); one experiment performed. FIG. 9D and FIG. 9E: one experiment performed. FIG. 10: n = 2; one experiment performed.

### TCGA Cohorts Correlation Analyses

The phenotype dataset (with survival outcomes) and the RNAseq dataset of each cancer cohort (the gene-level transcription estimates, as in log₂(x+1) transformed RSEM normalized count) were downloaded from UCSC Xena (https://xenabrowser.net/) where genes are mapped onto the human genome coordinates using UCSC Xena HUGO probeMap in the RNAseq dataset. Spearman correlation coefficient was calculated between TREM2 and each of the genes in each cancer cohort, accompanied with sample size and p values. The individual scatterplot of each of the top genes with TREM2 (with fitted linear lines) was generated with Spearman correlation in the plot.

The Kaplan-Meier curve for overall survival (OS) and relapse free survival (RFS) was generated for TREM2 dichotomized by 75% quantile of TREM2 expression into high/low TREM2 expression group. The log-rank test was applied to test the survival difference between high/low TREM2 expression.

### Single Cell RNAseq Analysis

Cellranger cell count was used to align samples to the reference mm10 genome and quantify reads. The Seurat package (Butler et al., 2018) in R was used for subsequent analysis. Cells with mitochondrial content greater than 12.5% and with less than 1100 genes were removed. Cells identified as doublets or mutliplets based on gene expression signatures, when more than one cell population-specific marker gene was highly expressed in one cell, were filtered out. Filtered data were normalized using a scaling factor of 10,000 , nUMI was regressed with a negative binomial model, and data was log transformed. The highly variable genes were selected using the FindVariableFeatures. Principal component analysis was performed using the top 3000 variable genes. Clustering was performed using the FindClusters function. UMAP was used to project cells into two dimensions using 20 first principal components. For myeloid/lymphoid cells re-clustering we chose clusters that were identified as myeloid/lymphoid cells. For these cells we performed normalization, found variable genes and performed PCA, UMAP and clustering as described above. All visualization was done with ggplot2 R package (Wickham, 2016), heatmaps were done with Phantasus website (https://artyomovlab.wustl.edu/phantasus/).

For the heatmap showing the magnitude of difference/similarity of each cluster, we averaged gene expression across all clusters within all the conditions in the TREM2 knockout experiment. To form the signature of the anti-TREM2 treatment experiment, we determined differential expression for each cluster versus all to find the most differentially expressed genes and, for each cluster, we used the 25 most differentially expressed genes to form the signature. Then, we used averaged expression of these 25 genes to form a heatmap comparing clusters from the anti-TREM2 experiment with clusters from the TREM2 knockout experiment.

TABLE 1

TREM2 Has Survival Prognostic Ability Independent of Other Myeloid Genes, Related to FIG. 13 . Multivariate survival analysis of survival outcomes in TNBC. Relapse-free survival (RFS) and overall survival (OS) are shown. TREM2 (in continuous scale) and a myeloid gene (indicated in the table) are included. Hazard ratio (95% Cl) and accompanying p value is reported.
Triple Negative Breast Cancer (TNBC)

| | Overall Survival | | Relapse Free Survival | |
|---|---|---|---|---|
| | hazard ratio (95%Cl) | Pvalue | hazard ratio (95%Cl) | Pvalue |
| TREM2 | 1.89 (1.07∼3.34) | 0.02711477 | 2.07 (1.16∼3.7) | 0.014398548 |
| APOE | 0.78 (0.49∼1.26) | 0.31973164 | 0.96 (0.58∼1.56) | 0.857608299 |
| TREM2 | 2.01 (1.16~3.47) | 0.01287235 | 3.43 (1.78~6.61) | 0.000228824 |
| FCGR1A | 0.71 (0.45∼1.13) | 0.14536141 | 0.46 (0.25∼0.85) | 0.0133663 |
| TREM2 | 1.77 (1.06∼2.94) | 0.02880634 | 2.63 (1.54∼4.51) | 0.000414618 |
| FCGR3A | 0.84 (0.54~1.31) | 0.44516957 | 0.59 (0.34∼1.03) | 0.063739833 |
| TREM2 | 1.59 (1.01∼2.5) | 0.04638629 | 2.25 (1.32∼3.83) | 0.00291724 |
| MERTK | 1.05 (0.69∼1.61) | 0.80851898 | 0.72 (0.45∼1.13) | 0.153190078 |

### REFERENCES

P. Allavena, M. Chieppa, G. Bianchi, G. Solinas, M. Fabbri, G. Laskarin, A. Mantovani Engagement of the mannose receptor by tumoral mucins activates an immune suppressive phenotype in human tumor-associated macrophage Clin. Dev. Immunol., 2010 (2010), p. 547179

E. Alspach, D.M. Lussier, A.P. Miceli, I. Kizhvatov, M. DuPage, A.M. Luoma, W. Meng, C.F. Lichti, E. Esaulova, A.N. Vomund, et al. MHC-II neoantigens shape tumour immunity and response to immunotherapy Nature, 574 (2019), pp. 696-701

S.P. Arlauckas, S.B. Garren, C.S. Garris, R.H. Kohler, J. Oh, M.J. Pittet, R. Weissleder Arg1 expression defines immunosuppressive subsets of tumor-associated macrophages Theranostics, 8 (2018), pp. 842-5854

A.A. Barkal, R.E. Brewer, M. Markovic, M. Kowarsky, S.A. Barkal, B.W. Zaro, V. Krishnan, J. Hatakeyama, O. Dorigo, L.J. Barkal, I.L. Weissman CD24 signalling through macrophage Siglec-10 is a target for cancer immunotherapy Nature, 572 (2019), pp. 92-396

S.K. Biswas, A. Mantovani Orchestration of metabolism by macrophages Cell Metab., 15 (2012), pp. 432-437

A. Bouchon, C. Hernandez-Munain, M. Cella, M. Colonna A DAP12-mediated pathway regulates expression of CC chemokine receptor 7 and maturation of human dendritic cells J. Exp. Med., 194 (2001), pp. 1111-1122

M.L. Broz, M.F. Krummel The emerging understanding of myeloid cells as partners and targets in tumor rejection Cancer Immunol. Res., 3 (2015), pp. 313-319

A. Butler, P. Hoffman, P. Smibert, E. Papalexi, R. Satija Integrating single-cell transcriptomic data across different conditions, technologies, and species Nat. Biotechnol., 36 (2018), pp. 411-420

L. Cassetta, J.W. Pollard Targeting macrophages: therapeutic approaches in cancer Nat. Rev. Drug Discov., 17 (2018), pp. 887-904

M. Cella, C. Buonsanti, C. Strader, T. Kondo, A. Salmaggi, M. Colonna Impaired differentiation of osteoclasts in TREM-2-deficient individuals J. Exp. Med., 198 (2003), pp. 645-651

C. Cochain, E. Vafadarnejad, P. Arampatzi, J. Pelisek, H. Winkels, K. Ley, D. Wolf, A.E. Saliba, A. Zernecke Single-Cell RNA-Seq Reveals the Transcriptional Landscape and Heterogeneity of Aortic Macrophages in Murine Atherosclerosis Circ. Res., 122 (2018), pp. 1661-1674

.L. Costa, M.L. Robinette, M. Bugatti, M.S. Longtine, B.N. Colvin, E. Lantelme, W. Vermi, M. Colonna, D.M. Nelson, M. Cella Two Distinct Myeloid Subsets at the Term Human Fetal-Maternal Interface Front. Immunol., 8 (2017), p. 1357

E. Elinav, R. Nowarski, C.A. Thaiss, B. Hu, C. Jin, R.A. Flavell Inflammation-induced cancer: crosstalk between tumours, immune cells and microorganisms Nat. Rev. Cancer, 13 (2013), pp. 759-771

R.A. Franklin, W. Liao, A. Sarkar, M.V. Kim, M.R. Bivona, K. Liu, E.G. Pamer, M.O. Li The cellular and molecular origin of tumor-associated macrophages Science, 344 (2014), pp. 921-925

G.J. Freeman, A.J. Long, Y. Iwai, K. Bourque, T. Chernova, H. Nishimura, L.J. Fitz, N. Malenkovich, T. Okazaki, M.C. Byrne, et al. Engagement of the PD-1 immunoinhibitory receptor by a novel B7 family member leads to negative regulation of lymphocyte activation J. Exp. Med., 192 (2000), pp. 1027-1034

S. Gilfillan, C.J. Chan, M. Cella, N.M. Haynes, A.S. Rapaport, K.S. Boles, D.M. Andrews, M.J. Smyth, M. Colonna DNAM-1 promotes activation of cytotoxic lymphocytes by nonprofessional antigen-presenting cells and tumors J. Exp. Med., 205 (2008), pp. 2965-2973

M.M. Gubin, E. Esaulova, J.P. Ward, O.N. Malkova, D. Runci, P. Wong, T. Noguchi, C.D. Arthur, W. Meng, E. Alspach, et al. High-Dimensional Analysis Delineates Myeloid and Lymphoid Compartment Remodeling during Successful Immune-Checkpoint Cancer Therapy Cell, 175 (2018), p. 1443

H. Harjunpää, S.J. Blake, E. Ahern, S. Allen, J. Liu, J. Yan, V. Lutzky, K. Takeda, A.R. Aguilera, C. Guillerey, et al. Deficiency of host CD96 and PD-1 or TIGIT enhances tumor immunity without significantly compromising immune homeostasis Oncolmmunology, 7 (2018), p. e1445949

J.B. Hibbs Jr., R.R. Taintor, Z. Vavrin Macrophage cytotoxicity: role for L-arginine deiminase and imino nitrogen oxidation to nitrite Science, 235 (1987), pp. 473-476

S. Hoves, C.H. Ooi, C. Wolter, H. Sade, S. Bissinger, M. Schmittnaegel, O. Ast, A.M. Giusti, K. Wartha, V. Runza, et al. Rapid activation of tumor-associated macrophages boosts preexisting tumor immunity J. Exp. Med., 215 (2018), pp. 859-876

R. Hughes, B.Z. Qian, C. Rowan, M. Muthana, I. Keklikoglou, O.C. Olson, S. Tazzyman, S. Danson, C. Addison, M. Clemons, et al. Perivascular M2 Macrophages Stimulate Tumor Relapse after Chemotherapy Cancer Res., 75 (2015), pp. 3479-3491

D.A. Hume, K.P. MacDonald Therapeutic applications of macrophage colony-stimulating factor-1 (CSF-1) and antagonists of CSF-1 receptor (CSF-1R) signaling Blood, 119 (2012), pp. 1810-1820

K. Iribarren, A. Buque, L. Mondragon, W. Xie, S. Levesque, J. Pol, L. Zitvogel, O. Kepp, G. Kroemer Anticancer effects of anti-CD47 immunotherapy in vivo Oncolmmunology, 8 (2018), p. 1550619

H. Ito, J.A. Hamerman TREM-2, triggering receptor expressed on myeloid cell-2, negatively regulates TLR responses in dendritic cells Eur. J. Immunol., 42 (2012), pp. 176-185

D.A. Jaitin, L. Adlung, C.A. Thaiss, A. Weiner, B. Li, H. Descamps, P. Lundgren, C. Bleriot, Z. Liu, A. Deczkowska, et al. Lipid-Associated Macrophages Control Metabolic Homeostasis in a Trem2-Dependent Manner Cell, 178 (2019), pp. 686-698.e14

Y. Lavin, S. Kobayashi, A. Leader, E.D. Amir, N. Elefant, C. Bigenwald, R. Remark, R. Sweeney, C.D. Becker, J.H. Levine, et al. Innate Immune Landscape in Early Lung Adenocarcinoma by Paired Single-Cell Analyses Cell, 169 (2017), pp. 750-765.e17

D.R. Leach, M.F. Krummel, J.P. Allison Enhancement of antitumor immunity by CTLA-4 blockade Science, 271 (1996), pp. 1734-1736

X.Y. Li, I. Das, A. Lepletier, V. Addala, T. Bald, K. Stannard, D. Barkauskas, J. Liu, A.R. Aguilera, K. Takeda, et al. CD155 loss enhances tumor suppression via combined host and tumor-intrinsic mechanisms J. Clin. Invest., 128 (2018), pp. 2613-2625

E.Y. Lin, A.V. Nguyen, R.G. Russell, J.W. Pollard Colony-stimulating factor 1 promotes progression of mammary tumors to malignancy J. Exp. Med., 193 (2001), pp. 727-740

M. Lo, H.S. Kim, R.K. Tong, T.W. Bainbridge, J.M. Vernes, Y. Zhang, Y.L. Lin, S. Chung, M.S. Dennis, Y.J. Zuchero, et al. Effector-attenuating Substitutions That Maintain Antibody Stability and Reduce Toxicity in Mice J. Biol. Chem., 292 (2017), pp. 3900-3908

A. Mantovani, F. Marchesi, A. Malesci, L. Laghi, P. Allavena Tumour-associated macrophages as treatment targets in oncology Nat. Rev. Clin. Oncol., 14 (2017), pp. 399-416

P.J. Murray, J.E. Allen, S.K. Biswas, E.A. Fisher, D.W. Gilroy, S. Goerdt, S. Gordon, J.A. Hamilton, L.B. Ivashkiv, T. Lawrence, et al. Macrophage activation and polarization: nomenclature and experimental guidelines Immunity, 41 (2014), pp. 14-20

L.S. Ojalvo, W. King, D. Cox, J.W. Pollard High-density gene expression analysis of tumor-associated macrophages from mouse mammary tumors Am. J. Pathol., 174 (2009), pp. 1048-1064

Q. Peng, S. Malhotra, J.A. Torchia, W.G. Kerr, K.M. Coggeshall, M.B. Humphrey TREM2- and DAP12-dependent activation of PI3K requires DAP10 and is inhibited by SHIP1 Sci. Signal., 3 (2010), p. ra38

P. Ramachandran, R. Dobie, J.R. Wilson-Kanamori, E.F. Dora, B.E.P. Henderson, N.T. Luu, J.R. Portman, K.P. Matchett, M. Brice, J.A. Marwick, et al. Resolving the fibrotic niche of human liver cirrhosis at single-cell level Nature, 575 (2019), pp. 512-518

M. Rashidian, M.W. LaFleur, V.L. Verschoor, A. Dongre, Y. Zhang, T.H. Nguyen, S. Kolifrath, A.R. Aref, C.J. Lau, C.P. Paweletz, et al. Immuno-PET identifies the myeloid compartment as a key contributor to the outcome of the antitumor response under PD-1 blockade Proc. Natl. Acad. Sci. USA, 116 (2019), pp. 16971-16980

C.H. Ries, M.A. Cannarile, S. Hoves, J. Benz, K. Wartha, V. Runza, F. Rey-Giraud, L.P. Pradel, F. Feuerhake, I. Klaman, et al. Targeting tumor-associated macrophages with anti-CSF-1R antibody reveals a strategy for cancer therapy Cancer Cell, 25 (2014), pp. 846-859

S.P. Schoenberger, R.E. Toes, E.I. van derVoort, R. Offringa, C.J. Melief T-cell help for cytotoxic T lymphocytes is mediated by CD40-CD40L interactions Nature, 393 (1998), pp. 480-483

R.D. Schreiber, L.J. Old, M.J. Smyth Cancer immunoediting: integrating immunity’s roles in cancer suppression and promotion Science, 331 (2011), pp. 1565-1570

V. Shankaran, H. Ikeda, A.T. Bruce, J.M. White, P.E. Swanson, L.J. Old, R.D. Schreiber IFNgamma and lymphocytes prevent primary tumour development and shape tumour immunogenicity Nature, 410 (2001), pp. 1107-1111

P. Sharma, J.P. Allison The future of immune checkpoint therapy Science, 348 (2015), pp. 56-61

F.D. Shi, M. Flodström, S.H. Kim, S. Pakala, M. Cleary, H.G. Ljunggren, N. Sarvetnick Control of the autoimmune response by type 2 nitric oxide synthase J. Immunol., 167 (2001), pp. 3000-3006

W. Song, B. Hooli, K. Mullin, S.C. Jin, M. Cella, T.K. Ulland, Y. Wang, R.E. Tanzi, M. Colonna Alzheimer’s disease-associated TREM2 variants exhibit either decreased or increased ligand-dependent activation Alzheimers Dement., 13 (2017), pp. 381-387

Q. Song, G.A. Hawkins, L. Wudel, P.C. Chou, E. Forbes, A.K. Pullikuth, L. Liu, G. Jin, L. Craddock, U. Topaloglu, et al. Dissecting intratumoral myeloid cell plasticity by single cell RNA-seq Cancer Med., 8 (2019), pp. 3072-3085

D. Stadlbauer, X. Zhu, M. McMahon, J.S. Turner, T.J. Wohlbold, A.J. Schmitz, S. Strohmeier, W. Yu, R. Nachbagauer, P.A. Mudd, et al. Broadly protective human antibodies that target the active site of influenza virus neuraminidase Science, 366 (2019), pp. 499-504

D.J. Stuehr, C.F. Nathan Nitric oxide. A macrophage product responsible for cytostasis and respiratory inhibition in tumor target cells J. Exp. Med., 169 (1989), pp. 1543-1555

X. Su, A.K. Esser, S.R. Amend, J. Xiang, Y. Xu, M.H. Ross, G.C. Fox, T. Kobayashi, V. Steri, K. Roomp, et al. Antagonizing Integrin β3 Increases Immunosuppression in Cancer Cancer Res., 76 (2016), pp. 3484-3495

S.L. Topalian, C.G. Drake, D.M. Pardoll Immune checkpoint blockade: a common denominator approach to cancer therapy Cancer Cell, 27 (2015), pp. 450-461

I.R. Turnbull, S. Gilfillan, M. Cella, T. Aoshi, M. Miller, L. Piccio, M. Hernandez, M. Colonna Cutting edge: TREM-2 attenuates macrophage activation J. Immunol., 177 (2006), pp. 3520-3524

T.K. Ulland, M. Colonna TREM2 - a key player in microglial biology and Alzheimer disease Nat. Rev. Neurol., 14 (2018), pp. 667-675

T.K. Ulland, W.M. Song, S.C. Huang, J.D. Ulrich, A. Sergushichev, W.L. Beatty, A.A. Loboda, Y. Zhou, N.J. Cairns, A. Kambal, et al. TREM2 Maintains Microglial Metabolic Fitness in Alzheimer’s Disease Cell, 170 (2017), pp. 649-663.e13

F. Veglia, M. Perego, D. Gabrilovich Myeloid-derived suppressor cells coming of age Nat. Immunol., 19 (2018), pp. 108-119

A. Veillette, J. Chen SIRPα-CD47 Immune Checkpoint Blockade in Anticancer Therapy Trends Immunol., 39 (2018), pp. 173-184

W. Vermi, A. Micheletti, S. Lonardi, C. Costantini, F. Calzetti, R. Nascimbeni, M. Bugatti, M. Codazzi, P.C. Pinter, K. Schakel, et al. slanDCs selectively accumulate in carcinoma-draining lymph nodes and marginate metastatic cells Nat. Commun., 5 (2014), p. 3029

Y. Wang, M. Cella, K. Mallinson, J.D. Ulrich, K.L. Young, M.L. Robinette, S. Gilfillan, G.M. Krishnan, S. Sudhakar, B.H. Zinselmeyer, et al. TREM2 lipid sensing sustains the microglial response in an Alzheimer’s disease model Cell, 160 (2015), pp. 1061-1071

Y. Wang, T.K. Ulland, J.D. Ulrich, W. Song, J.A. Tzaferis, J.T. Hole, P. Yuan, T.E. Mahan, Y. Shi, S. Gilfillan, et al. TREM2-mediated early microglial response limits diffusion and toxicity of amyloid plaques J. Exp. Med., 213 (2016), pp. 667-675

E.C.E. Wang, Z. Dai, A.W. Ferrante, C.G. Drake, A.M. Christiano A Subset of TREM2(+) Dermal Macrophages Secretes Oncostatin M to Maintain Hair Follicle Stem Cell Quiescence and Inhibit Hair Growth Cell stem cell, 24 (2019), pp. 654-669.e6

H. Wickham ggplot2-Elegant Graphics for Data Analysis Springer (2016)

K.R. Wiehagen, N.M. Girgis, D.H. Yamada, A.A. Smith, S.R. Chan, I.S. Grewal, M. Quigley, R.I. Verona Combination of CD40 Agonism and CSF-1R Blockade Reconditions Tumor-Associated Macrophages and Drives Potent Antitumor Immunity Cancer Immunol. Res., 5 (2017), pp. 1109-1121

S.B. Willingham, J.P. Volkmer, A.J. Gentles, D. Sahoo, P. Dalerba, S.S. Mitra, J. Wang, H. Contreras-Trujillo, R. Martin, J.D. Cohen, etal. The CD47-signal regulatory protein alpha (SIRPa) interaction is a therapeutic target for human solid tumors Proc. Natl. Acad. Sci. USA, 109 (2012), pp. 6662-6667

S.R. Woo, M.E. Turnis, M.V. Goldberg, J. Bankoti, M. Selby, C.J. Nirschl, M.L. Bettini, D.M. Gravano, P. Vogel, C.L. Liu, et al. Immune inhibitory molecules LAG-3 and PD-1 synergistically regulate T-cell function to promote tumoral immune escape Cancer Res., 72 (2012), pp. 917-927

K. Wu, D.E. Byers, X. Jin, E. Agapov, J. Alexander-Brett, A.C. Patel, M. Cella, S. Gilfilan, M. Colonna, D.L. Kober, et al. TREM-2 promotes macrophage survival and lung disease after respiratory viral infection J. Exp. Med., 212 (2015), pp. 681-697

L. Zhong, X.F. Chen, T. Wang, Z. Wang, C. Liao, Z. Wang, R. Huang, D. Wang, X. Li, L. Wu, et al. Soluble TREM2 induces inflammatory responses and enhances microglial survival J. Exp. Med., 214 (2017), pp. 597-607

### Example 2: TREM2 in Tumors

TREM2 in tumors can be targeted by a number of methods and anti-TREM2 compositions. An anti-human TREM2 can include 21E10 (Piccio et al. Identification of soluble TREM-2 in the cerebrospinal fluid and its association with multiple sclerosis and CNS inflammation Brain, Volume 131, Issue 11, November 2008, Pages 3081-3091) or anti-TREM-2 mAb 29E3 (probably sister clones). Other anti-human TREM2 can be anti-human TREM2, F(ab′)2 anti-TREM-2 (Bouchon etal., A DAP12-mediated Pathway Regulates Expression of CC Chemokine Receptor 7 and Maturation of Human Dendritic cells, J. Exp. Med. Volume 194, Number 8, Oct. 15, 2001, 1111-1122).

Other examples can include anti-mouse TREM2 (178) (Turnbull et al. Cutting edge: TREM-2 attenuates Macrophage activation J Immunol Sep. 15, 2006, 177 (6) 3520-3524).

As shown here, TREM2-deficient mice are less susceptible to transplantable models of MCA-derived sarcoma, MC38 colon carcinoma, and PyMT breast cancer (see e.g., FIG. 16).

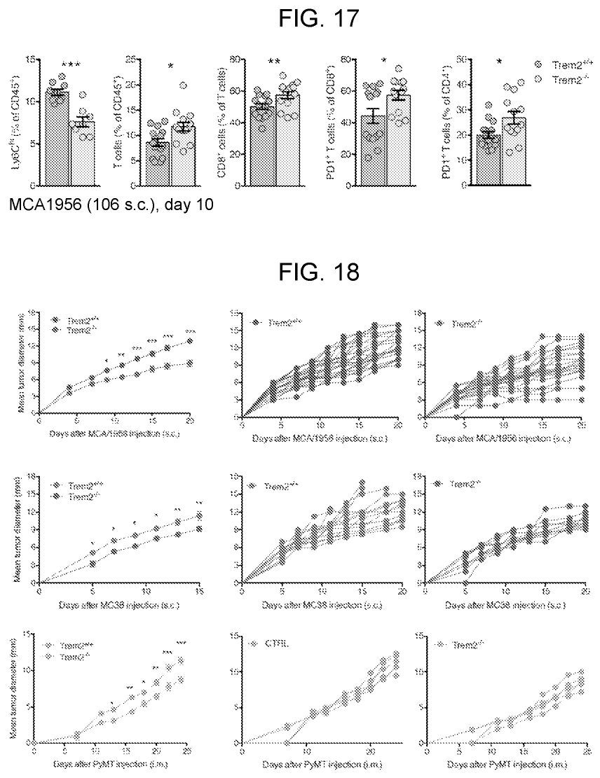

TREM2-deficiency is associated with reduced Ly6Chi myeloid cells and increased CD8+ and PD1+ T cells (see e.g., FIG. 17).

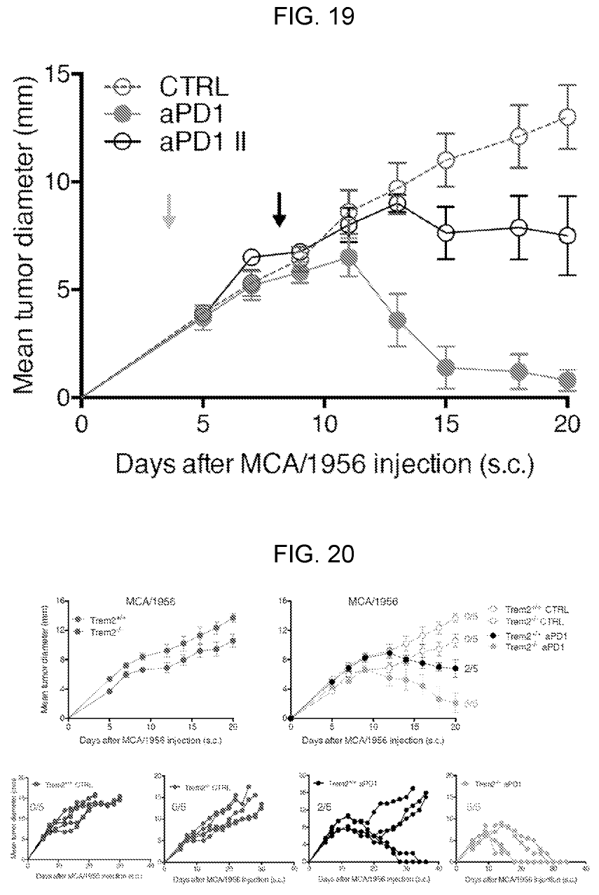

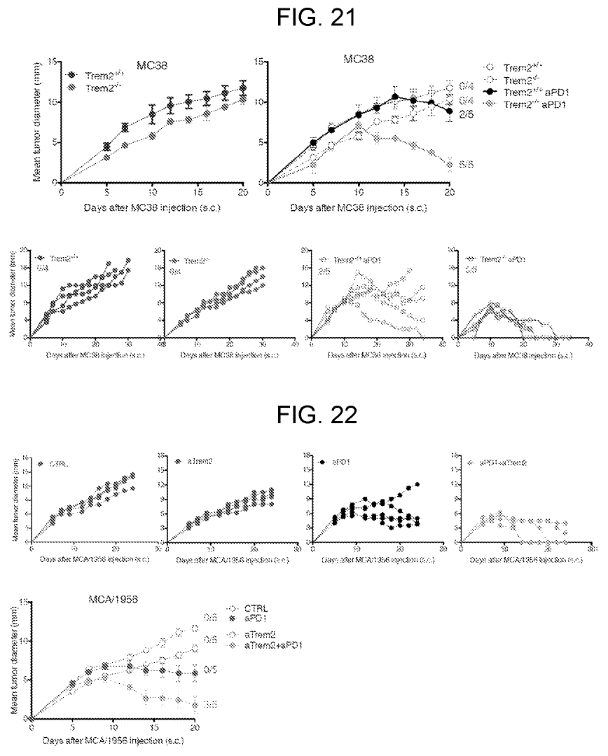

TREM2-deficient mice are less susceptible to transplantable models of MCA-derived sarcoma, MC38 colon carcinoma, and PyMT breast cancer (see e.g., FIG. 18). Anti-PD-1 treatment protocol (see e.g., FIG. 19). TREM2-deficiency enhances anti-PD-1 response in MCA sarcoma (see e.g., FIG. 20). TREM2-deficiency enhances anti-PD-1 response in MC38 colon carcinoma (see e.g., FIG. 21).

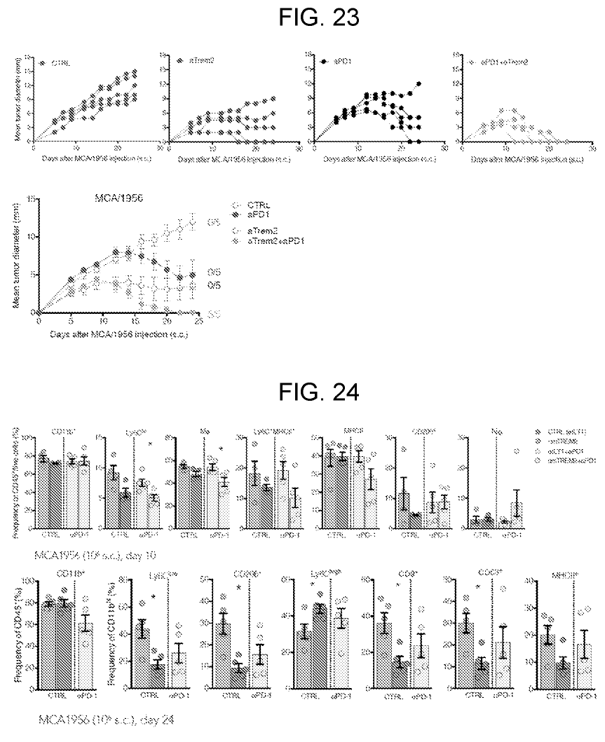

Anti-TREM2-treatment is protective and enhances anti-PD-1 response in MCA sarcoma (see e.g., FIG. 22). Anti-TREM2-treatment is protective and enhances anti-PD-1 response in MCA sarcoma (see e.g., FIG. 23). Anti-TREM2-treatment remodels the myeloid compartment (see e.g., FIG. 24).

### Summary

TREM2 is expressed in tumor-associated macrophages. TREM2 deficiencyisassociated with reduced growth and enhanced anti-PD-1 response in MCA-sarcomaand MC38-colon carcinoma. Anti-TREM2 treatment is protective in MCA-sarcoma and boosts anti-PD-1 response in MCA-sarcoma. TREM2-deficiency is associated with a reduced infiltrate of Ly6C^{high} myeloid cells. Anti-TREM2 treatment induced a skewing in the myeloid compartment and promotes T cell activation. TREM2 expression is associated with a reduced overall survival and relapse free survival in CRC and TNBC. TREM2 expression correlates with TAM genes in cancer patients.

### Example 3: Anti-Tumor Effect of Human TREM2 Blockade in A Humanized Mouse Model

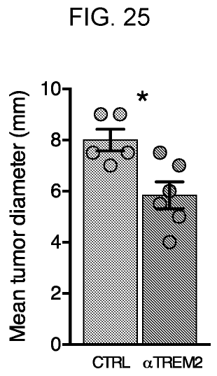

Targeting mouse TREM2 reduced tumor growth in a methylcholanthrene (MCA) sarcoma preclinical model. Thus, those studies were extended to human TREM2. We previously generated the 21 E10 mAb specific for human TREM2 (Song, W.M. et al. Humanized TREM2 mice reveal microglia-intrinsic and -extrinsic effects of R47H polymorphism. J Exp Med 215, 745-760 (2018)). We generated a recombinant version of this antibody in which the Fab′ region of the mAb was grafted onto a mouse IgG1 Fc backbone with the LALAPG mutations that abrogate Fc effector functions. Thus, the antibody was expected to specifically modulate TREM2 without causing ADCC or ADP and potentially inhibit TREM2-ligand interaction. To test the antibody in vivo, we generated BAC transgenic mice expressing the common variant (CV) of human TREM2 (TREM2^{cv} mice) and crossed them with Trem2^{-/-} mice to eliminate endogenous TREM2. To test whether human TREM2 blockade is beneficial in tumor bearing mice, we injected TREM2^{cv} mice with the MCA1956 cell line and treated them with anti-hTREM2. It was observed that anti-hTREM2 treatment reduced tumor growth (FIG. 25), indicating that blockade of human TREM2 is a promising strategy to be pursued using a strategy similar to that used for mouse TREM2.

### Example 4: Preclinical Model Carrying the Human TREM2 Gene

We have shown that anti-mouse TREM2 blockade augments anti-tumor immune responses by reshaping the myeloid immune infiltrate. We also have initial data indicating that anti-human TREM2 mAb 21E10 delays MCA1956 growth in mice expressing human TREM2 in place of mouse TREM2. Given these premises, it is believed that TREM2 targeting can be extended to a preclinical model carrying the human TREM2 gene and using a recombinant anti-human TREM2.

### Anti-Tumor Effect of Anti-Human TREM2 in TREM2^{CV} Mice

We have developed mice that lack endogenous TREM2 and carry the common variant of the human TREM2 gene (hTREM2^{CV}). We will inject hTREM2^{CV} and controls with a MCA-derived cell line and treat them with a recombinant Fc mutated anti-hTREM2 mAb 21E10 developed in our lab. We will follow the same treatment protocol that we set up for the experiments with the anti-mouse TREM2 antibody. We will measure tumor growth and analyze the immune infiltrate at different time-points by flow cytometry. We will then test combinations of anti-hTREM2 mAb 21E10 with anti-PD1, anti-CTLA4, and doxorubicin to determine whether the effect of human TREM2 blockade synergizes with other treatments enhancing anti-tumor immune responses. To gain insight into the impact of anti-human TREM2 treatment on tumor-associated macrophages, we will perform single cell RNA-seq on the tumor immune infiltrate, as described above.

Given the data showing anti-human TREM2 mAb 21E10 delays MCA1956 growth in hTREM2^{CV} mice, it is expected that anti-hTREM2 can have a beneficial effect in the MCA model and drive the generation of a less immunosuppressive tumor microenvironment. It is presently believed that mAb 21E10 will synergize with immune checkpoint therapy and doxorubicin.

1. A method of suppressing tumor growth in a subject having cancer, comprising:
administering a TREM2 inhibiting agent and an immunotherapy to a subject in an amount effective to suppress tumor growth; wherein
the TREM2 inhibiting agent has TREM2 inhibiting function;
the TREM2 inhibiting agent comprises an antibody or a functional fragment or variant thereof; and
the TREM2 inhibiting agent results in a reduction or loss of immune cell effector function.

2. The method of claim 1, wherein the TREM2 inhibiting agent comprises a mutant or dysfunctional Fc region or Fc domain, resulting in a loss or reduced effector function.

3. The method of claim 2, wherein the TREM2 inhibiting agent comprises a loss of function mutation in the Fc region or Fc domain to result in a reduction or loss of Fc effector function.

4. The method of claim 1, wherein the TREM2 inhibiting agent comprises an Fc mutation comprising an amino acid addition, insertion, deletion, substitution, or combination thereof.

5. The method of claim 1, wherein the TREM2 inhibiting agent comprises an addition of one or more glycans in an Fc domain.

6. The method of claim 1, wherein the TREM2 inhibiting agent comprises a variable region of a heavy chain grafted onto a constant region backbone mutated in an Fc domain.

7. The method of claim 1, wherein the TREM2 inhibiting agent comprises LALAPG mutations that abrogate Fc effector functions.

8. The method of claim 1, wherein the TREM2 inhibiting agent comprises an anti-TREM2 antibody construct, Fc-fusion antibody-like protein, an anti-TREM2 antibody, recombinant anti-TREM2 antibody or protein, a functional portion or fragment thereof, a fusion protein, scFv, peptide, diabody, unibody, or a functional fragment, variant, or mutant, thereof, or small molecule having TREM2 inhibiting or blocking function.

9. The method of claim 8, wherein the TREM2 inhibiting agent comprises an anti-TREM2 mAb.

10. The method of claim 8, wherein the TREM2 inhibiting agent comprises an Fc-mutated anti-TREM2 monoclonal antibody (mAb).

11. The method of claim 8, wherein the TREM2 inhibiting agent is a recombinant form of an anti-TREM2 mAb.

12. The method of claim 8, wherein the TREM2 inhibiting agent comprises 21 E10 mAb or mAb 178, or a functional fragment or variant thereof, specific for human TREM2.

13. The method of claim 8, wherein the TREM2 inhibiting agent comprises a mutation comprising an amino acid addition, insertion, deletion, substitution, or combination thereof.

14. The method of claim 1, wherein the TREM2 inhibiting agent comprises an Fc mutation, wherein the Fc mutation prevents or reduces effector function; recognition by Fc receptors; recognition by complement or depletes antibody fix complement; induces antibody-dependent cellular cytotoxicity; or antibody-dependent phagocytosis.

15. The method of claim 1, wherein the TREM2 inhibiting agent targets, inhibits, prevents, reduces, or blocks TREM2 function.

16. The method of claim 1, wherein the TREM2 inhibiting agent modifies tumor-infiltrating myeloid cells to maintain an environment hospitable to immune cells, optionally, T-cells.

17. The method of claim 1, wherein the TREM2 inhibiting agent has TREM2 blocking function and a loss of Fc effector function.

18. The method of claim 1, wherein the TREM2 inhibiting agent recruits T-cells.

19. The method of claim 1, wherein an effective amount or a therapeutically effective amount of the TREM2 inhibiting agent is an amount sufficient to induce immunostimulatory macrophages or reduce immunosuppressive macrophages.

20. The method of claim 1, wherein the amount or a therapeutically effective amount of the TREM2 inhibiting agent is an amount sufficient to make tumor microenvironment hospitable to T-cells; recruit T-cells to a tumor microenvironment; have loss or reduction of effector function; maintain immune system; or recruit immune cells.

21. The method of claim 1, wherein the amount of a therapeutically effective amount of the TREM2 inhibiting agent results in enhanced immunostimulation; prevention of cytokine storm in checkpoint blockade therapy; prevention of cytokine release syndrome; reduced checkpoint immunotherapy resistance; improving T-cell response; or enhanced checkpoint immunotherapy efficacy compared to a subject prior to receiving or a subject not receiving a TREM2 inhibiting agent therapy.

22. The method of claim 1, wherein the amount or a therapeutically effective amount of the TREM2 inhibiting agent prevents, targets, inhibits, blocks, or reduces TREM2 function, signaling, or activity, but does not kill or substantially deplete or kill macrophages or myeloid cells.

23. The method of claim 22, wherein macrophages are depleted compared to macrophages contacted with TREM2 inhibiting agent having a functional Fc.

24. The method of claim 1, wherein the TREM2 inhibiting agent does not cause antibody-dependent cellular cytotoxicity (ADCC) or antibody-dependent phagocytosis (ADP) and inhibits TREM2-ligand interaction.

25. The method of claim 1, wherein the TREM2 inhibiting agent prevents, blocks, or reduces TREM2 function, signaling, or activity and does not substantially kill myeloid or macrophages or reduce an amount of myeloid or macrophage cells compared to a TREM2 inhibiting agent having Fc effector function.

26. The method of claim 1, wherein the TREM2 inhibiting agent induces a skewing or increase in the ratio of immunostimulating myeloid cells to immunosuppressive myeloid cells in the myeloid compartment and promotes T cell activation.

27. The method of claim 26, wherein
MRC1 (CD206) is a correlative marker of immunosuppressive activity; or
iNOS (NOS2) is a correlative marker of immunostimulatory activity.

28. The method of claim 1, wherein the administering the TREM2 inhibiting agent results in
reduced Ly6Chi myeloid cells and increased CD8+ and PD1+ T cells; or
increased IFNλ-producing CD8+ T cells and TNFα-producing CD4+ T cells.

29. The method of claim 1, wherein the TREM2 inhibiting agent changes macrophage populations infiltrating a tumor and wherein CX₃CR1⁺ and MRC1⁺ macrophage subsets declined and subsets expressing potentially immunostimulatory molecules were induced.

30. The method of claim 1, wherein the immunotherapy is selected from a checkpoint immunotherapy or CAR-T.

31. The method of claim 30, wherein the checkpoint immunotherapy is a checkpoint blockade therapy.

32. The method of claim 30, wherein the checkpoint immunotherapy is checkpoint inhibitor therapy selected from anti-PD-1.

33. The method of claim 1, wherein the immunotherapy is selected from CAR-T.

34. The method of claim 1, wherein the TREM2 inhibiting agent targets immunosuppressive myeloid cells.

35. The method of claim 1, wherein the immune cell is an immunosuppressive myeloid cell or an immunostimulatory myeloid cell.

36. The method of claim 1, wherein the immune cell is an immunostimulatory myeloid cell selected from type 1 dendritic cells (DC1s) or M1-like IFN-γ-induced macrophages.

37. The method of claim 1, wherein the cancer is associated with a microenvironment infiltrated by macrophages expressing TREM2.

38. The method of claim 37, wherein the cancer is selected from sarcoma, progressors, colorectal carcinoma (CRC), breast cancer, or triple-negative breast cancer (TNBC).

39. The method of claim 1, wherein the TREM2 inhibiting agent is a TREM2 neutralizing antibody or functional fragment or variant thereof expressed on a cell.

40. The method of claim 1, wherein a TCR is ectopically expressed on a cell.

41. The method of claim 1, wherein the TREM2 inhibiting agent is a TREM2 neutralizing antibody or functional fragment or variant thereof expressed on a CAR-T cell.

42. A method of reducing myeloid-induced immune cell suppression comprising blocking TREM2 on a myeloid cell comprising contacting a TREM2 inhibiting agent to the myeloid cell, wherein the TREM2 inhibiting agent has TREM2 binding activity or TREM2 blocking function and a reduction or loss of Fc effector function.

43. The method of claim 42, wherein TREM2 expression is associated with a reduced overall survival and relapse free survival in CRC or TNBC.

44. A method of blocking TREM2 function and recruiting immune cells in a subject or cells comprising administering a TREM2 inhibiting agent to the subject or the cells of the subject receiving, having received, or to receive immunotherapy, wherein the TREM2 inhibiting agent comprises anti-TREM2 activity and reduced immune cell Fc effector function.

45. A method of reducing checkpoint immunotherapy resistance in a subject or cells of a subject in need thereof comprising administering a TREM2 inhibiting agent to the subject receiving, having received, or to receive immunotherapy.

46. A method of enhancing immunotherapy efficacy in a subject receiving, having received, or to receive immunotherapy comprising administering a TREM2 inhibiting agent to the subject or cells of a subject, wherein the TREM2 inhibiting agent modifies tumor-infiltrating myeloid cells to maintain an environment hospitable to immune cells.

47. A method of reshaping or remodeling of intratumoral, tumor-associated macrophage infiltrate population, comprising administering a TREM2 inhibiting agent and immunotherapy to a subject.

48. A method of treating a subject, wherein the subject has cancer or is suspected of having cancer comprising:
measuring an amount of TREM2 in a sample; and
if TREM2 is elevated compared to control, the subject is predicted to have a poor prognosis; or
if TREM2 is elevated compared to control, the subject is treated with a TREM2 inhibiting agent and immunotherapy.
